(19) United States
(12) Patent Application Publication
(10) Pub. No.: US 2009/0123918 A1
(43) Pub. Date: May 14, 2009

(54) Human Protooncogene TRG and Protein Encoded Therein

(76) Inventors: Hyun-Kee Kim, Seoul (KR); Jin-Woo Kim, Seoul (KR)

Correspondence Address: SUGHRUE MION, PLLC, 2100 PENNSYLVANIA AVENUE, N.W., SUITE 800, WASHINGTON, DC, 20037, US

(21) Appl. No.: 11/910,006

(22) PCT Filed: Mar. 30, 2006
(86) PCT No.: PCT/KR06/01175

§ 371 Date: Sep. 28, 2007

(30) Foreign Application Priority Data
Mar. 30, 2005 (KR) 10-2005-0026252

(51) Int. Cl. C12Q 1/68; C07H 21/04; C07K 14/82

(52) U.S. Cl. 435/6; 536/23.5; 530/350; 435/320.1

(57) ABSTRACT

Disclosed are a protooncogene and a protein encoded by the same. The protooncogene of the present invention, known to be involved in human carcinogenesis, may be effectively used for diagnosing various cancers including uterine cancer, leukemia, lymphoma, colon cancer, lung cancer, skin cancer, etc.

### TECHNICAL FIELD

The present invention relates to a novel protooncogene exhibiting an ability to induce carcinogenesis and cancer metastasis, and a protein encoded by the same.

### BACKGROUND ART

Generally, it has been known that the higher animals, including human, have approximately 30,000 genes, but only approximately 15% of the genes are expressed in each subject. Accordingly, it was found that all phenomena of life, namely development, differentiation, homeostasis, responses to stimulus, control of cell cycle, aging and apoptosis (a programmed cell death), etc. were determined depending on what genes are selected and expressed (Liang, P. and A. B. Pardee, Science 257: 967-971, 1992).

The pathological phenomena such as oncogenesis are induced by the genetic variation, resulting in changed expression of genes. Accordingly, it is thought that the comparison of gene expressions between different cells is a basic and fundamental approach to understand various biological mechanisms. For example, the mRNA differential display method proposed by Liang and Pardee (Liang, P. and A. B. Pardee, Science 257: 967-971, 1992) has been now effectively used for searching tumor suppressor genes, genes relevant to cell cycle regulation, and transcriptional regulatory genes relevant to apoptosis, etc., and also widely employed for specifying correlations of the various genes that appear only in one cell.

Putting together the various results of oncogenesis, it has been reported that various genetic changes such as loss of specific chromosomal heterozygosity, activation of protooncogenes, and inactivation of other tumor suppressor genes including the p53 gene were accumulated in the tumor tissues, resulting in development of human tumors (Bishop, J. M., Cell 64: 235-248, 1991; Hunter, T., Cell 64: 249-270, 1991). Also, it was reported that 10 to 30% of the cancer was induced if protooncogenes are activated by amplifying the protooncogenes, and therefore the activation of protooncogenes plays an important role in the etiological studies of many cancers. Accordingly, there have been attempts to specify the role.

Accordingly, the present inventors found that a mechanism for generating cervical cancer was studied at a protooncogene level, and therefore the protooncogene, named a human transformation-related gene (TRG), showed a specifically increased level of expression only in the cancer cell. The protooncogene may be effectively used for diagnosing, preventing and treating various cancers such as uterine cancer, leukemia, lymphoma, colon cancer, lung cancer, skin cancer, etc.

### DISCLOSURE OF INVENTION

Accordingly, the present invention is designed to solve the problems of the prior art, and therefore it is an object of the present invention to provide a novel protooncogene and their fragments.

It is another object of the present invention to provide a recombinant vector containing the protooncogene and their fragments; and a microorganism transformed by the recombinant vector.

It is still another object of the present invention to provide a protein encoded by the protooncogene; and its fragments.

It is still another object of the present invention to provide a kit for diagnosing cancer, including the protooncogene or its fragments.

It is yet another object of the present invention to provide a kit for diagnosing cancer, including the protein or its fragments.

In order to accomplish one of the above objects, the present invention provides a protooncogene having a DNA sequence selected from the group consisting of SEQ ID NO: 1; SEQ ID NO: 5; SEQ ID NO: 9; SEQ ID NO: 13; SEQ ID NO: 17; SEQ ID NO: 21; SEQ ID NO: 25; SEQ ID NO: 29; SEQ ID NO: 33; SEQ ID NO: 37; SEQ ID NO: 41; SEQ ID NO: 45; SEQ ID NO: 49; SEQ ID NO: 53; SEQ ID NO: 57; and SEQ ID NO: 61, and fragments thereof.

According to another of the above objects, the present invention provides a recombinant vector containing the protooncogene or its fragments; and a microorganism transformed by the recombinant vector.

According to still another of the above objects, the present invention provides a protein having an amino acid sequence selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; SEQ ID NO: 34; SEQ ID NO: 38; SEQ ID NO: 42; SEQ ID NO: 46; SEQ ID NO: 50; SEQ ID NO: 54; SEQ ID NO: 58; and SEQ ID NO: 62, and fragments thereof, the protein and the fragments thereof being encoded by the protooncogenes, respectively.

According to still another of the above objects, the present invention provides a kit for diagnosing cancer including the protooncogene or its fragments.

According to still another of the above objects, the present invention provides a kit for diagnosing cancer including the protooncoprotein or its fragments.

According to still another of the above objects, the present invention provides an anti-sense gene which has a DNA sequence complementary to the entire or partial sequence of mRNA transcribed from the protooncoprotein or its fragments and binds to the mRNA to suppress expression of the protooncoprotein or its fragments.

According to yet another of the above objects, the present invention provides an anti-cancer and anti-metastasis composition including the anti-sense gene as an active component.

Hereinafter, preferable embodiments of the present invention will be described in detail referring to the accompanying drawings.

1. TRG3

The protooncogene, a human transformation-related gene 3, of the present invention (hereinafter, referred to as TRG3) has a 1,703-bp full-length DNA sequence set forth in SEQ ID NO: 1.

In the DNA sequence of SEQ ID NO: 1, an open reading frame corresponding to nucleotide sequence positions from 113 to 1522 (1520-1522: a stop codon) is a full-length protein coding region, and an amino acid sequence derived from the protein coding region is set forth in SEQ ID NO: 2 and contains 469 amino acids (hereinafter, referred to as “a TRG3 protein”).

The DNA sequence of SEQ ID NO: 1 has been deposited with Accession No. AY189688 in the GenBank database of U.S. National Institutes of Health (NIH) (Scheduled Release Date: Apr. 8, 2005), and the DNA base sequence result revealed that its DNA sequence was similar to that of the Homo sapiens CGI-51 protein gene deposited with Accession No. BC011681 in the database. From this study result, it was however found that the TRG3 protooncogene is highly expressed in various human tumors including the uterine cancer, while its expression is significantly reduced in various normal tissues.

A protein expressed from the protooncogene of the present invention contains 469 amino acids and has an amino acid sequence set forth in SEQ ID NO: 2 and a molecular weight of approximately 52 kDa.

2. TRG4

The protooncogene, a human transformation-related gene 4, of the present invention (hereinafter, referred to as TRG4) has a 2,576-bp full-length DNA sequence set forth in SEQ ID NO: 5.

In the DNA sequence of SEQ ID NO: 5, an open reading frame corresponding to nucleotide sequence positions from 87 to 482 (480-482: a stop codon) is a full-length protein coding region, and an amino acid sequence derived from the protein coding region is set forth in SEQ ID NO: 6 and contains 131 amino acids (hereinafter, referred to as “a TRG4 protein”).

The DNA sequence of SEQ ID NO: 5 has been deposited with Accession No. AY189690 in the GenBank database of U.S. National Institutes of Health (NIH) (Scheduled Release Date: Apr. 8, 2005), and the DNA base sequence result revealed that its DNA sequence was similar to that of the Homo sapiens nucleoredoxin (NXN) gene deposited with Accession No. NM_{—}022463 in the database, but their protein sequences were completely different to each other. From this study result, it was however found that the TRG4 protooncogene is highly expressed in various human tumors including the uterine cancer, while its expression is significantly reduced in various normal tissues.

A protein expressed from the protooncogene of the present invention contains 131 amino acids and has an amino acid sequence set forth in SEQ ID NO: 6 and a molecular weight of approximately 14 kDa.

3. TRG5

The protooncogene, a human transformation-related gene 5, of the present invention (hereinafter, referred to as TRG5) has a 1,334-bp full-length DNA sequence set forth in SEQ ID NO: 9.

In the DNA sequence of SEQ ID NO: 9, an open reading frame corresponding to nucleotide sequence positions from 88 to 1,092 (1,090-1,092: a stop codon) is a full-length protein coding region, and an amino acid sequence derived from the protein coding region is set forth in SEQ ID NO: 10 and contains 334 amino acids (hereinafter, referred to as “a TRG5 protein”).

The DNA sequence of SEQ ID NO: 9 has been deposited with Accession No. AY189689 in the GenBank database of U.S. National Institutes of Health (NIH) (Scheduled Release Date: Apr. 8, 2005), and the DNA base sequence result revealed that its DNA sequence was similar to that of the Homo sapiens lactate dehydrogenase B (LDHB) gene deposited with Accession No. NM_{—}002300 in the database. From this study result, it was however found that the TRG5 protooncogene is highly expressed in various human tumors including the uterine cancer, while its expression is significantly reduced in various normal tissues.

A protein expressed from the protooncogene of the present invention contains 334 amino acids and has an amino acid sequence set forth in SEQ ID NO: 10 and a molecular weight of approximately 37 kDa.

4. TRG6

The protooncogene, a human transformation-related gene 6, of the present invention (hereinafter, referred to as TRG6) has a 3,309-bp full-length DNA sequence set forth in SEQ ID NO: 13. In the DNA sequence of SEQ ID NO: 13, an open reading frame corresponding to nucleotide sequence positions from 233 to 481 (479-481: a stop codon) is a full-length protein coding region, and an amino acid sequence derived from the protein coding region is set forth in SEQ ID NO: 14 and contains 82 amino acids (hereinafter, referred to as “a TRG6 protein”).

The DNA sequence of SEQ ID NO: 13 has been deposited with Accession No. AY191222 in the GenBank database of U.S. National Institutes of Health (NIH) (Scheduled Release Date: Apr. 8, 2005), and the DNA base sequence result revealed that some of its DNA sequence was similar to that of the RP11-175D17 clone on Chromosome 9 deposited with Accession No. AL354928 in the database. From this study result, it was however found that the TRG6 protooncogene is highly expressed in various human tumors including the uterine cancer, while its expression is significantly reduced in various normal tissues.

A protein expressed from the protooncogene of the present invention contains 82 amino acids and has an amino acid sequence set forth in SEQ ID NO: 14 and a molecular weight of approximately 9 kDa.

5. TRG7

The protooncogene, a human transformation-related gene 7, of the present invention (hereinafter, referred to as TRG7) has a 1,334-bp full-length DNA sequence set forth in SEQ ID NO: 17.

In the DNA sequence of SEQ ID NO: 17, an open reading frame corresponding to nucleotide sequence positions from 42 to 1,422 (1,420-1,422: a stop codon) is a full-length protein coding region, and an amino acid sequence derived from the protein coding region is set forth in SEQ ID NO: 18 and contains 175 amino acids (hereinafter, referred to as “a TRG7 protein”).

The DNA sequence of SEQ ID NO: 17 has been deposited with Accession No. AY191223 in the GenBank database of U.S. National Institutes of Health (NIH) (Scheduled Release Date: Apr. 8, 2005), and the DNA base sequence result revealed that its DNA sequence was similar to that of the Homo sapiens cDNA FLJ90076 fis, clone HEMBA1004444 deposited with Accession No. AK074557 in the database. From this study result, it was however found that the TRG7 protooncogene is highly expressed in various human tumors including the uterine cancer, while its expression is significantly reduced in various normal tissues.

A protein expressed from the protooncogene of the present invention contains 175 amino acids and has an amino acid sequence set forth in SEQ ID NO: 18 and a molecular weight of approximately 20 kDa.

6. TRG9

The protooncogene, a human transformation-related gene 9, of the present invention (hereinafter, referred to as TRG9) has a 1,582-bp full-length DNA sequence set forth in SEQ ID NO: 21.

In the DNA sequence of SEQ ID NO: 21, an open reading frame corresponding to nucleotide sequence positions from 17 to 1,576 (1,574-1,576: a stop codon) is a full-length protein coding region, and an amino acid sequence derived from the protein coding region is set forth in SEQ ID NO: 22 and contains 519 amino acids (hereinafter, referred to as “a TRG9 protein”).

The DNA sequence of SEQ ID NO: 21 has been deposited with Accession No. AY272044 in the GenBank database of U.S. National Institutes of Health (NIH) (Scheduled Release Date: Mar. 31, 2005), and the DNA base sequence result revealed that its DNA sequence was similar to that of the Homo sapiens sorting nexin 2 (SNX2) deposited with Accession No. NM_{—}003100 in the database. From this study result, it was however found that the TRG9 protooncogene is highly expressed in various human tumors including the uterine cancer, while its expression is significantly reduced in various normal tissues.

A protein expressed from the protooncogene of the present invention contains 519 amino acids and has an amino acid sequence set forth in SEQ ID NO: 22 and a molecular weight of approximately 58 kDa.

7. TRG10

The protooncogene, a human transformation-related gene 10, of the present invention (hereinafter, referred to as TRG10) has a 3,979-bp full-length DNA sequence set forth in SEQ ID NO: 25.

In the DNA sequence of SEQ ID NO: 25, an open reading frame corresponding to nucleotide sequence positions from 1,100 to 1,270 (1,268-1,270: a stop codon) is a full-length protein coding region, and an amino acid sequence derived from the protein coding region is set forth in SEQ ID NO: 26 and contains 56 amino acids (hereinafter, referred to as “a TRG10 protein”).

The DNA sequence of SEQ ID NO: 25 has been deposited with Accession No. AY277593 in the GenBank database of U.S. National Institutes of Health (NIH) (Scheduled Release Date: Mar. 31, 2005), and the DNA base sequence result revealed that its DNA sequence was similar to that of the Homo sapiens bone morphogenetic protein receptor, type II (serine/threonine kinase) (BMPR2), transcriptional variant 2 gene deposited with Accession No. NM_{—}033346 in the database. From this study result, it was however found that the TRG10 protooncogene is highly expressed in various human tumors including the uterine cancer, while its expression is significantly reduced in various normal tissues.

A protein expressed from the protooncogene of the present invention contains 56 amino acids and has an amino acid sequence set forth in SEQ ID NO: 26 and a molecular weight of approximately 6 kDa.

8. TRG11

The protooncogene, a human transformation-related gene 11, of the present invention (hereinafter, referred to as TRG11) has a 235-bp full-length DNA sequence set forth in SEQ ID NO: 29.

In the DNA sequence of SEQ ID NO: 29, an open reading frame corresponding to nucleotide sequence positions from 26 to 214 (227-229: a stop codon) is a full-length protein coding region, and an amino acid sequence derived from the protein coding region is set forth in SEQ ID NO: 30 and contains 62 amino acids (hereinafter, referred to as “a TRG11 protein”).

The DNA sequence of SEQ ID NO: 29 has been deposited with Accession No. AY277594 in the GenBank database of U.S. National Institutes of Health (NIH) (Scheduled Release Date: Mar. 31, 2005), and the DNA base sequence result revealed that its DNA sequence was similar to that of the Homo sapiens IMAGE: 5258564 gene clone deposited with Accession No. BC022205 in the database. From this study result, it was however found that the TRG11 protooncogene is highly expressed in various human tumors including the uterine cancer, while its expression is significantly reduced in various normal tissues.

A protein expressed from the protooncogene of the present invention contains 62 amino acids and has an amino acid sequence set forth in SEQ ID NO: 30 and a molecular weight of approximately 7 kDa.

9. TRG12

The protooncogene, a human transformation-related gene 12, of the present invention (hereinafter, referred to as TRG12) has a 510-bp full-length DNA sequence set forth in SEQ ID NO: 33.

In the DNA sequence of SEQ ID NO: 33, an open reading frame corresponding to nucleotide sequence positions from 80 to 475 (473-475: a stop codon) is a full-length protein coding region, and an amino acid sequence derived from the protein coding region is set forth in SEQ ID NO: 34 and contains 131 amino acids (hereinafter, referred to as “a TRG12 protein”).

The DNA sequence of SEQ ID NO: 33 has been deposited with Accession No. AY277595 in the GenBank database of U.S. National Institutes of Health (NIH) (Scheduled Release Date: Mar. 31, 2005), and the DNA base sequence result revealed that its DNA sequence was similar to that of the Homo sapiens DNA59497 MY047 (UNQ577) gene deposited with Accession No. AY358674 in the database. From this study result, it was however found that the TRG12 protooncogene is highly expressed in various human tumors including the uterine cancer, while its expression is significantly reduced in various normal tissues.

A protein expressed from the protooncogene of the present invention contains 131 amino acids and has an amino acid sequence set forth in SEQ ID NO: 34 and a molecular weight of approximately 14 kDa.

10. TRG13

The protooncogene, a human transformation-related gene 13, of the present invention (hereinafter, referred to as TRG13) has a 1,301-bp full-length DNA sequence set forth in SEQ ID NO: 37.

In the DNA sequence of SEQ ID NO: 37, an open reading frame corresponding to nucleotide sequence positions from 18 to 1,193 (1,191-1,193: a stop codon) is a full-length protein coding region, and an amino acid sequence derived from the protein coding region is set forth in SEQ ID NO: 38 and contains 391 amino acids (hereinafter, referred to as “a TRG13 protein”).

The DNA sequence of SEQ ID NO: 37 has been deposited with Accession No. AY277596 in the GenBank database of U.S. National Institutes of Health (NIH) (Scheduled Release Date: Mar. 31, 2005), and the DNA base sequence result revealed that its DNA sequence was similar to those of the Homo sapiens MGC11349 gene and the Homo sapiens MGC11349 gene, deposited with Accession No. BC002940 and BC012729 in the database, respectively. Functions of the genes remain to be known. From this study result, it was however found that the TRG13 protooncogene is highly expressed in various human tumors including the uterine cancer, while its expression is significantly reduced in various normal tissues.

A protein expressed from the protooncogene of the present invention contains 391 amino acids and has an amino acid sequence set forth in SEQ ID NO: 38 and a molecular weight of approximately 40 kDa.

11. TRG14

The protooncogene, a human transformation-related gene 14, of the present invention (hereinafter, referred to as TRG14) has a 1,206-bp full-length DNA sequence set forth in SEQ ID NO: 41.

In the DNA sequence of SEQ ID NO: 41, an open reading frame corresponding to nucleotide sequence positions from 18 to 1,202 (1,200-1,202: a stop codon) is a full-length protein coding region, and an amino acid sequence derived from the protein coding region is set forth in SEQ ID NO: 42 and contains 394 amino acids (hereinafter, referred to as “a TRG14 protein”).

The DNA sequence of SEQ ID NO: 41 has been deposited with Accession No. AY277597 in the GenBank database of U.S. National Institutes of Health (NIH) (Scheduled Release Date: Mar. 31, 2005), and the DNA base sequence result revealed that its DNA sequence was similar to that of the Homo sapiens N-myc downstream regulated gene 1 (NDRG1) gene deposited with Accession No. NM_{—}006096 in the database. From this study result, it was however found that the TRG14 protooncogene is highly expressed in various human tumors including the uterine cancer, while its expression is significantly reduced in various normal tissues.

A protein expressed from the protooncogene of the present invention contains 394 amino acids and has an amino acid sequence set forth in SEQ ID NO: 42 and a molecular weight of approximately 43 kDa.

12. TRG15

The protooncogene, a human transformation-related gene 15, of the present invention (hereinafter, referred to as TRG15) has a 1,104-bp full-length DNA sequence set forth in SEQ ID NO: 45.

In the DNA sequence of SEQ ID NO: 45, an open reading frame corresponding to nucleotide sequence positions from 1 to 1,104 (1,102-1,104: a stop codon) is a full-length protein coding region, and an amino acid sequence derived from the protein coding region is set forth in SEQ ID NO: 46 and contains 367 amino acids (hereinafter, referred to as “a TRG15 protein”).

The DNA sequence of SEQ ID NO: 45 has been deposited with Accession No. AY277598 in the GenBank database of U.S. National Institutes of Health (NIH) (Scheduled Release Date: Mar. 31, 2005), and the DNA base sequence result revealed that its DNA sequence was similar to that of the Homo sapiens FLJ20758 protein gene deposited with Accession No. NM_{—}017952 in the database. Functions of the FLJ20758 protein gene remain to be known. From this study result, it was however found that the TRG15 protooncogene is highly expressed in various human tumors including the uterine cancer, while its expression is significantly reduced in various normal tissues.

A protein expressed from the protooncogene of the present invention contains 367 amino acids and has an amino acid sequence set forth in SEQ ID NO: 46 and a molecular weight of approximately 42 kDa.

13. TRG16

The protooncogene, a human transformation-related gene 16, of the present invention (hereinafter, referred to as TRG16) has a 1,064-bp full-length DNA sequence set forth in SEQ ID NO: 49.

In the DNA sequence of SEQ ID NO: 49, an open reading frame corresponding to nucleotide sequence positions from 92 to 1,064 (1,062-1,064: a stop codon) is a full-length protein coding region, and an amino acid sequence derived from the protein coding region is set forth in SEQ ID NO: 50 and contains 324 amino acids (hereinafter, referred to as “a TRG16 protein”).

The DNA sequence of SEQ ID NO: 49 has been deposited with Accession No. AY277601 in the GenBank database of U.S. National Institutes of Health (NIH) (Scheduled Release Date: Mar. 31, 2005), and the DNA base sequence result revealed that its DNA sequence was similar to that of the Homo sapiens pp 9320 mRNA gene deposited with Accession No. AF318376 in the database. Functions of the pp 9320 gene remain to be known. From this study result, it was however found that the TRG16 protooncogene is highly expressed in various human tumors including the uterine cancer, while its expression is significantly reduced in various normal tissues.

A protein expressed from the protooncogene of the present invention contains 324 amino acids and has an amino acid sequence set forth in SEQ ID NO: 50 and a molecular weight of approximately 36 kDa.

14. TRG17

The protooncogene, a human transformation-related gene 17, of the present invention (hereinafter, referred to as TRG17) has a 432-bp full-length DNA sequence set forth in SEQ ID NO: 53.

In the DNA sequence of SEQ ID NO: 53, an open reading frame corresponding to nucleotide sequence positions from 1 to 408 (406-408: a stop codon) is a full-length protein coding region, and an amino acid sequence derived from the protein coding region is set forth in SEQ ID NO: 54 and contains 135 amino acids (hereinafter, referred to as “a TRG17 protein”). The DNA sequence of SEQ ID NO: 53 has been deposited with Accession No. AY277599 in the GenBank database of U.S. National Institutes of Health (NIH) (Scheduled Release Date: Mar. 31, 2005), and the DNA base sequence result revealed that its DNA sequence was similar to those of the Homo sapiens MGC5309 (MGC5309) gene and the Homo sapiens MGC5309 gene, deposited with Accession No. NM_{—}032286 and BC003353 in the database, respectively. From this study result, it was however found that the TRG17 protooncogene is highly expressed in various human tumors including the uterine cancer, while its expression is significantly reduced in various normal tissues.

A protein expressed from the protooncogene of the present invention contains 135 amino acids and has an amino acid sequence set forth in SEQ ID NO: 54 and a molecular weight of approximately 16 kDa.

15. TRG18

The protooncogene, a human transformation-related gene 18, of the present invention (hereinafter, referred to as TRG18) has a 1,141-bp full-length DNA sequence set forth in SEQ ID NO: 57.

In the DNA sequence of SEQ ID NO: 57, an open reading frame corresponding to nucleotide sequence positions from 20 to 1,141 (1,139-1,141: a stop codon) is a full-length protein coding region, and an amino acid sequence derived from the protein coding region is set forth in SEQ ID NO: 58 and contains 373 amino acids (hereinafter, referred to as “a TRG18 protein”). The DNA sequence of SEQ ID NO: 57 has been deposited with Accession No. AY277600 in the GenBank database of U.S. National Institutes of Health (NIH) (Scheduled Release Date: Mar. 31, 2005), and the DNA base sequence result revealed that its DNA sequence was similar to that of the Homo sapiens mRNA for PEX3 protein deposited with Accession No. AJ131389 in the database. From this study result, it was however found that the TRG18 protooncogene is highly expressed in various human tumors including the uterine cancer, while its expression is significantly reduced in various normal tissues.

A protein expressed from the protooncogene of the present invention contains 373 amino acids and has an amino acid sequence set forth in SEQ ID NO: 58 and a molecular weight of approximately 42 kDa.

16. TRG20

The protooncogene, a human transformation-related gene 20, of the present invention (hereinafter, referred to as TRG20) has a 449-bp full-length DNA sequence set forth in SEQ ID NO: 61.

In the DNA sequence of SEQ ID NO: 61, an open reading frame corresponding to nucleotide sequence positions from 42 to 449 (447-449: a stop codon) is a full-length protein coding region, and an amino acid sequence derived from the protein coding region is set forth in SEQ ID NO: 62 and contains 135 amino acids (hereinafter, referred to as “a TRG20 protein”). The DNA sequence of SEQ ID NO: 61 has been deposited with Accession No. AY453397 in the GenBank database of U.S. National Institutes of Health (NIH) (Scheduled Release Date: Mar. 31, 2005), and the DNA base sequence result revealed that its DNA sequence was similar to those of the Homo sapiens hypothetical protein MGC5309 gene and the Homo sapiens hypothetical protein MGC5309 gene, deposited with Accession No. NM_{—}032286 and BC003353 in the database, respectively. From this study result, it was however found that the TRG20 protooncogene is highly expressed in various human tumors including the uterine cancer, while its expression is significantly reduced in various normal tissues.

A protein expressed from the protooncogene of the present invention contains 135 amino acids and has an amino acid sequence set forth in SEQ ID NO: 62 and a molecular weight of approximately 16 kDa.

Meanwhile, because of degeneracy of codons, or considering preference of codons for living organisms to express the protooncogenes, the protooncogenes of the present invention may be variously modified in coding regions without changing an amino acid sequence of the oncogenic protein expressed from the coding region, and also be variously modified or changed in a region except the coding region within a range that does not affect the gene expression. Such a modified gene is also included in the scope of the present invention. Accordingly, the present invention also includes polynucleotides having substantially the same DNA sequences as the above-mentioned protooncogenes; and fragments thereof. The term “substantially the same polynucleotide” means a DNA sequence having a sequence homology of at least 80%, preferably at least 90%, and the most preferably at least 95% with DNA of SEQ ID NO: 1 encoding the same translated protein product as SEQ ID NO: 2.

Also, one or more amino acids may be substituted, added or deleted even in the amino acid sequences of the proteins of the present invention within a range that does not affect functions of the proteins, and only some of the proteins may be used depending on their usage. Such a modified amino acid sequence is also included in the scope of the present invention. Accordingly, the present invention also includes polypeptides having substantially the same amino acid sequences as the oncogenic proteins; and fragments thereof. The term “substantially the same polypeptide” means a polypeptide having sequence homology of at least 80%, preferably at least 90%, and the most preferably at least 95%.

The protooncogenes and the proteins of the present invention may be separated from human cancer tissues, or be also synthesized according to the known methods for synthesizing DNA or peptide. Also, the genes prepared thus may be inserted into a vector for expression in the microorganisms, already known in the art, to obtain expression vectors, and then the expression vectors may be introduced into suitable host cells, for example Escherichia coli, yeast cells, etc. DNA of the genes of the present invention may be replicated in a large quantity or its protein may be produced in a commercial quantity in such a transformed host.

Upon constructing the expression vectors, expression regulatory sequences such as a promoter and a terminator, autonomously replicating sequences, secretion signals, etc. may be suitably selected and combined depending on kinds of the host cells that produce the protooncogenes or the proteins.

The genes of the present invention are proved to be strong oncogenes capable of developing the uterine cancer since it was revealed the genes were rarely expressed in a normal exocervical tissue, but overexpressed in a cervical cancer tissue and a uterine cancer cell line in the analysis methods such as a northern blotting, etc. In addition to epithelial tissues such as the cervical cancer, the protooncogenes of the present invention are highly expressed in other cancerous tumors such as leukemia, colon cancer, etc. Accordingly, the protooncogenes of the present invention are considered to be common oncogenes in the various oncogenesis, and may be effectively used for diagnosing the various cancers, producing the transformed animals and for anti-sense gene therapy, etc.

For example, a method for diagnosing the cancer using the protooncogenes includes a step of determining whether or not a subject has the protooncogenes of the present invention by detecting the protooncogenes using the various methods known in the art after the entire or partial protooncogenes are used as proves to hybridize with nucleic acid extracted from the subject's body fluids. It can be easily confirmed that the genes are present in the tissue samples by using the probes labeled with a radioactive isotope, an enzyme, etc. Accordingly, the present invention also provides kits for diagnosing the cancer including all or some of the protooncogenes.

The transformed animals may be obtained by introducing the protooncogenes of the present invention into mammals, for example rodents such as a rat, and the protooncogenes are preferably introduced at the fertilized egg stage prior to at least 8-cell stage. The transformed animals prepared thus may be effectively used for searching carcinogenic substances or anticancer substances such as antioxidants.

The present invention also provides an anti-sense gene that is effective in the gene therapy. In this application, the term “anti-sense gene” is a polynucleotide having a DNA sequence complementary to the partial or entire sequence of mRNA that is transcribed from the protooncogene or its fragments, and may be used to prevent and treat the cancer caused by the expression of the protooncogene by introducing into patients the DNA sequence having a sequence that can bind to a protein binding region of the mRNA to destruct an open reading frame (ORF) of the protooncogene. The present invention also provides a method for treating or preventing the cancers or the cancer metastasis by administrating a therapeutically effective amount of the anti-sense gene of the present invention to patients

In the anti-sense gene therapy of the present invention, the anti-sense gene of the present invention is administered to the patients using the conventional manners to prevent the expression of the protooncogenes. For example, an anti-sense oligodeoxynucleotide (ODN) was mixed with a poly-L-lysine derivative by means of electrostatic attraction and the resultant mixture was intravenously administered to the patients according to the method of the disclosure (J. S. Kim et al., J. Controlled Release 53: 175-182, 1998).

Also, the pharmaceutical composition of the present invention includes an anti-cancer composition comprising the anti-sense gene of the present invention in conjunction with a pharmaceutically available carrier, vehicle, or optionally other additives, within the sprite and scope of the present invention. The pharmaceutical composition of the present invention is preferably formulated into an injection dosage form.

An amount of the actually administered anti-sense gene is varied according to various related factors such as conditions to be treated, a route of administration, age and body weight of patients, severity of the condition, etc.

The proteins derived from the protooncogenes of the present invention may be effectively used as a diagnostic tool to produce antibodies. The antibodies of the present invention may be produced as the monoclonal or polyclonal antibodies according to the conventional methods known in the art using the proteins having the amino acid sequences expressed from the protooncogenes of the present invention; or their fragments, and therefore those antibodies may be used to diagnose the cancer by determining whether or not the proteins are expressed in the body fluid samples of the subject using the method known in the art, for example an enzyme linked immunosorbent assay (ELISA), a radioimmunoassay (RIA), a sandwich assay, western blotting or immunoblotting on the polyacrylamide gel, etc.

Also, the protooncogenes of the present invention may be used to establish cancer cell lines that can grow in an uncontrolled manner, and this cell line may be, for example, produced from a tumorous tissue developed in the back of a nude mouse using fibroblast cell transfected with the protooncogene. This cancer cell line may be effectively used for searching anticancer agents, etc.

Hereinafter, the present invention will be described in detail referring to preferred examples, but the description proposed herein is just a preferable example for the purpose of illustrations only, not intended to limit the scope of the invention.

### BRIEF DESCRIPTION OF THE DRAWINGS

These and other features, aspects, and advantages of preferred embodiments of the present invention will be more fully described in the following detailed description, taken accompanying drawings. In the drawings:

FIGS. 1 to 16 are diagrams showing results of the differential display reverse transcription-polymerase chain reaction (DDRT-PCR) to determine whether or not an H20-121 DNA fragment (FIG. 1); an H93-811 DNA fragment (FIG. 2); an H117-321 DNA fragment (FIG. 3); an H38-211 DNA fragment (FIG. 4); an H38-621 DNA fragment (FIG. 5); an H96 DNA fragment (FIG. 6); an H94 DNA fragment (FIG. 7); an H42 DNA fragment (FIG. 8); an H109 DNA fragment (FIG. 9); an H119 DNA fragment (FIG. 10); an H201 DNA fragment (FIG. 11); an H1151 DNA fragment (FIG. 12); an H132 DNA fragment (FIG. 13); an H141 DNA fragment (FIG. 14); an H181 DNA fragment (FIG. 15); and an H134 DNA fragment (FIG. 16) are expressed in a normal exocervical tissue, a cervical tumor tissue, a metastatic lymph node tumor tissue and a CUMC-6 cancer cell, respectively;

FIGS. 17 to 32 are diagrams showing northern blotting results to determine whether or not TRG3 (top of FIG. 17); TRG4 (top of FIG. 18); TRG5 (top of FIG. 19); TRG6 (top of FIG. 20); TRG7 (top of FIG. 21); TRG9 (top of FIG. 22); TRG10 (top of FIG. 23); TRG11 (top of FIG. 24); TRG12 (top of FIG. 25) TRG13 (top of FIG. 26); TRG14 (top of FIG. 27); TRG15 (top of FIG. 28); TRG16 (top of FIG. 29); TRG17 (top of FIG. 30); TRG18 (top of FIG. 31); and TRG20 (top of FIG. 32) protooncogenes of the present invention are expressed in a normal exocervical tissue, a uterine cancer tissue, a metastatic cervical lymph node tissue and a cervical cancer cell line, respectively; and bottoms of FIGS. 17 to 32 are diagrams showing northern blotting results obtained by hybridizing the same samples as in the tops of FIGS. 17 to 32 with β-actin probe, respectively.

FIGS. 33 to 48 are diagrams showing northern blotting results to determine whether or not TRG3 (FIG. 33); TRG4 (FIG. 34); TRG5 (FIG. 35); TRG6 (FIG. 36); TRG7 (FIG. 37); TRG9 (FIG. 38); TRG10 (FIG. 39); TRG11 (FIG. 40); TRG12 (FIG. 41); TRG13 (FIG. 42); TRG14 (FIG. 43); TRG15 (FIG. 44); TRG16 (FIG. 45); TRG17 (FIG. 46); TRG18 (FIG. 47); and TRG20 (FIG. 48) protooncogenes of the present invention are expressed in a normal human 12-lane multiple tissue, respectively; and bottoms of FIGS. 33 to 48 are diagrams showing northern blotting results obtained by hybridizing the same samples as in the tops of FIGS. 33 to 48 with β-actin probe, respectively.

FIGS. 49 to 64 are diagrams showing northern blotting results to determine whether or not TRG3 (FIG. 49); TRG4 (FIG. 50); TRG5 (FIG. 51); TRG6 (FIG. 52); TRG7 (FIG. 53); TRG9 (FIG. 54); TRG10 (FIG. 55); TRG11 (FIG. 56); TRG12 (FIG. 57); TRG13 (FIG. 58); TRG14 (FIG. 59); TRG15 (FIG. 60); TRG16 (FIG. 61); TRG17 (FIG. 62); TRG18 (FIG. 63); and TRG20 (FIG. 64) protooncogenes of the present invention are expressed in human cancer cell lines, respectively; and bottoms of FIGS. 49 to 64 are diagrams showing northern blotting results obtained by hybridizing the same samples as in the tops of FIGS. 49 to 64 with β-actin probe, respectively.

FIGS. 65 to 80 are diagrams showing results of sodium dodecyl sulfate-polyacrylamide gel electrophoresis (SDS-PAGE) to determine sizes of the proteins expressed before and after L-arabinose induction after TRG3 (FIG. 65); TRG4 (FIG. 66); TRG5 (FIG. 67); TRG6 (FIG. 68); TRG7 (FIG. 69); TRG9 (FIG. 70); TRG10 (FIG. 71); TRG11 (FIG. 72); TRG12 (FIG. 73) TRG13 (FIG. 74); TRG14 (FIG. 75); TRG15 (FIG. 76); TRG16 (FIG. 77); TRG17 (FIG. 78); TRG18 (FIG. 79); and TRG20 (FIG. 80) protooncogenes of the present invention are transformed into Escherichia coli, respectively.

### BEST MODES FOR CARRYING OUT THE INVENTION

Hereinafter, preferred embodiments of the present invention will be described in detail with reference to the accompanying drawings.

However, the description proposed herein is just a preferable example for the purpose of illustrations only, not intended to limit the scope of the invention.

### Example 1

### Cultivation of Tumor Cell and Separation of Total RNA

(Step 1) Cultivation of Tumor Cell

In order to conduct the mRNA differential display method, a normal exocervical tissue was obtained from a patient suffering from an uterine myoma who has been subject to hysterectomy, and a primary cervical tumor tissue and a metastatic lymph node tumor tissue were obtained from an uterine cancer patient who has not been previously subject to the anticancer chemotherapy and/or radiation therapy during the surgery operation. CUMC-6 (Kim, J. W. et al., Gynecol. Oncol. 62: 230-240, 1996) was used as the human cervical cancer cell line in the differential display method.

The cells obtained from the obtained tissues and the CUMC-6 cell line were grown in Waymouth's MB 752/1 media (Gibco) containing 2 mM glutamine, 100 IU/ml penicillin, 100 μg/ml streptomycin and 10% fetal bovine serum (Gibco, U.S.). The culture cells used in this experiment are at the exponentially growing stage, and the cells showing a viability of at least 95% in a trypan blue staining were used herein (Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the normal exocervical tissue, the primary cervical tumor tissue, the metastatic lymph node tumor tissue and the CUMC-6 cell, each obtained in Step 1, using the commercially available system RNeasy total RNA kit (Qiagen Inc., Germany). DNA contaminants were removed from the RNA samples using the message clean kit (GenHunter Corp., Brookline, Mass., U.S.).

### Example 2

### Differential Display Reverse Transcription-Polymerase Chain Reaction (DDRT-PCR)

The differential display reverse transcription was carried out using a slightly modified reverse transcription-polymerase chain reaction (RT-PCR) proposed by Liang, P. and A. B. Pardee.

2-1. TRG3

At first, reverse transcription was conducted on 0.2 μg of the total RNA obtained in Step 1 of Example 1 using an anchored primer H-T11A (5′-AAGCTTTTTTTTTTTA-3′, RNAimage kit, Genhunter, Cor., MA, U.S.) having a DNA sequence set forth in SEQ ID NO: 3 as the anchored oligo-dT primer.

Then, a PCR reaction was carried out in the presence of 0.5 mM [α-³⁵S] dATP (1200 Ci/mmole) using the same anchored primer and the primer H-AP20 (5′-AAGCTTGTTGTGC-3′) having a DNA sequence set forth in SEQ ID NO: 4 out of the random 5′-13-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The PCR-amplified fragment was dissolved in a 6% polyacrylamide sequencing gel, and then a position of a differentially expressed band was determined using the autoradiography.

A 258-base pair (bp) band with H20-121 cDNA (Base positions from 1,342 to 1,599 of SEQ ID NO: 1) was cut out from the dried gel. The extracted gel was heated for 15 minutes to elute the H20-121 cDNA, and then the PCR reaction was repeated with the same primers under the same condition as described above to re-amplify the H20-121 cDNA, except that [α-³⁵S]-labeled dATP (1200 Ci/mmole) and 20 μM dNTP were not used herein.

2-2. TRG4

The PCR reaction was repeated in the same manner as in Example 2-1, except that an anchored primer H-T11G (5′-AAGCTTTTTTTTTTTG-3′, RNAimage kit, Genhunter, Cor., MA, U.S.) set forth in SEQ ID NO: 7 and a primer H-AP9 (5′-AAGCTTCATTCCG-3′) set forth in SEQ ID NO: 8 were used herein.

A 393-base pair (bp) band with H93-811 cDNA (Base positions from 2,086 to 2,478 of SEQ ID NO: 5) was cut out from the dried gel. The extracted gel was heated for 15 minutes to elute the H93-811 cDNA, and then the PCR reaction was repeated with the same primers under the same condition as described above to re-amplify the H93-811 cDNA, except that [α-³⁵S]-labeled dATP (1200 Ci/mmole) and 20 μM dNTP were not used herein.

2-3. TRG5

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 11 and a primer H-AP11 (5′-AAGCTTCGGGTAA-3′) having a DNA sequence set forth in SEQ ID NO: 12 were used herein.

A 292-base pair (bp) band with H117-321 cDNA (Base positions from 933 to 1,224 of SEQ ID NO: 9) was cut out from the dried gel. The extracted gel was heated for 15 minutes to elute the H117-321 cDNA, and then the PCR reaction was repeated with the same primers under the same condition as described above to re-amplify the H117-321 cDNA, except that [α-³⁵S]-labeled dATP (1200 Ci/mmole) and 20 μM dNTP were not used herein.

2-4. TRG6

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 15 and a primer H-AP38 (5′-AAGCTTCCAGTGC-3′) having a DNA sequence set forth in SEQ ID NO: 16 were used herein.

A 311-base pair (bp) band with H38-211 cDNA (Base positions from 2,823 to 3,133 of SEQ ID NO: 13) was cut out from the dried gel. The extracted gel was heated for 15 minutes to elute the H38-211 cDNA, and then the PCR reaction was repeated with the same primers under the same condition as described above to re-amplify the H38-211 cDNA, except that [α-³⁵S]-labeled dATP (1200 Ci/mmole) and 20 μM dNTP were not used herein.

2-5. TRG7

The PCR reaction was repeated in the same manner as in Example 2-1, except that an anchored primer H-T11G (5′-AAGCTTTTTTTTTTTG-3′, RNAimage kit, Genhunter, Cor., MA, U.S.) having a DNA sequence set forth in SEQ ID NO: 19 and a primer H-AP38 (5′-AAGCTTCCAGTGC-3′) having a DNA sequence set forth in SEQ ID NO: 20 were used herein.

A 292-base pair (bp) band with H38-621 cDNA (Base positions from 1,404 to 1,695 of SEQ ID NO: 17) was cut out from the dried gel. The extracted gel was heated for 15 minutes to elute the H38-621 cDNA, and then the PCR reaction was repeated with the same primers under the same condition as described above to re-amplify the H38-621 cDNA, except that [α-³⁵S]-labeled dATP (1200 Ci/mmole) and 20 μM dNTP were not used herein.

2-6. TRG9

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 23 and a primer H-AP9 (5′-AAGCTTCATTCCG-3′) having a DNA sequence set forth in SEQ ID NO: 24 were used herein.

A 275-base pair (bp) band with H96 cDNA (Base positions from 1,225 to 1,499 of SEQ ID NO: 21) was cut out from the dried gel. The extracted gel was heated for 15 minutes to elute the H96 cDNA, and then the PCR reaction was repeated with the same primers under the same condition as described above to re-amplify the H96 cDNA, except that [α-³⁵S]-labeled dATP (1200 Ci/mmole) and 20 μM dNTP were not used herein.

2-7. TRG10

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 27 and a primer H-AP9 (5′-AAGCTTCATTCCG-3′) having a DNA sequence set forth in SEQ ID NO: 28 were used herein.

A 352-base pair (bp) band with H94 cDNA (Base positions from 3,528 to 3,879 of SEQ ID NO: 25) was cut out from the dried gel. The extracted gel was heated for 15 minutes to elute the H94 cDNA, and then the PCR reaction was repeated with the same primers under the same condition as described above to re-amplify the H94 cDNA, except that [α-³⁵S]-labeled dATP (1200 Ci/mmole) and 20 μM dNTP were not used herein.

2-8. TRG11

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 31 and a primer H-AP4 (5′-AAGCTTCTCAACG-3′) having a DNA sequence set forth in SEQ ID NO: 32 were used herein.

A 147-base pair (bp) band with H42 cDNA (Base positions from 83 to 229 of SEQ ID NO: 29) was cut out from the dried gel. The extracted gel was heated for 15 minutes to elute the H42 cDNA, and then the PCR reaction was repeated with the same primers under the same condition as described above to re-amplify the H42 cDNA, except that [α-³⁵S]-labeled dATP (1200 Ci/mmole) and 20 μM dNTP were not used herein.

2-9. TRG12

The PCR reaction was repeated in the same manner as in Example 2-1, except that an anchored primer H-T11G (5′-AAGCTTTTTTTTTTTG-3′, RNAimage kit, Genhunter, Cor., MA, U.S.) having a DNA sequence set forth in SEQ ID NO: 35 and a primer H-AP10 (5′-AAGCTTCCACGTA-3′) having a DNA sequence set forth in SEQ ID NO: 36 were used herein.

A 212-base pair (bp) band with H109 cDNA (Base positions from 284 to 495 of SEQ ID NO: 33) was cut out from the dried gel. The extracted gel was heated for 15 minutes to elute the H109 cDNA, and then the PCR reaction was repeated with the same primers under the same condition as described above to re-amplify the H109 cDNA, except that [α-³⁵S]-labeled dATP (1200 Ci/mmole) and 20 μM dNTP were not used herein.

2-10. TRG13

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 39 and a primer H-AP11 (5′-AAGCTTCGGGTAA-3′) having a DNA sequence set forth in SEQ ID NO: 40 were used herein.

A 232-base pair (bp) band with H119 cDNA (Base positions from 1,004 to 1,235 of SEQ ID NO: 37) was cut out from the dried gel. The extracted gel was heated for 15 minutes to elute the H119 cDNA, and then the PCR reaction was repeated with the same primers under the same condition as described above to re-amplify the H119 cDNA, except that [α-³⁵S]-labeled dATP (1200 Ci/mmole) and 20 μM dNTP were not used herein.

2-11. TRG14

The PCR reaction was repeated in the same manner as in Example 2-1, except that an anchored primer H-T11G (5′-AAGCTTTTTTTTTTTG-3′, RNAimage kit, Genhunter, Cor., MA, U.S.) having a DNA sequence set forth in SEQ ID NO: 43 and a primer H-AP20 (5′-AAGCTTGTTGTGC-3′) having a DNA sequence set forth in SEQ ID NO: 44 were used herein.

A 195-base pair (bp) band with H201 cDNA (Base positions from 902 to 1,096 of SEQ ID NO: 41) was cut out from the dried gel. The extracted gel was heated for 15 minutes to elute the H201 cDNA, and then the PCR reaction was repeated with the same primers under the same condition as described above to re-amplify the H201 cDNA, except that [α-³⁵S]-labeled dATP (1200 Ci/mmole) and 20 μM dNTP were not used herein.

2-12. TRG15

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 47 and a primer H-AP15 (5′-AAGCTTACGCAAC-3′) having a DNA sequence set forth in SEQ ID NO: 48 were used herein.

A 252-base pair (bp) band with H151 cDNA (Base positions from 848 to 1,099 of SEQ ID NO: 45) was cut out from the dried gel. The extracted gel was heated for 15 minutes to elute the H151 cDNA, and then the PCR reaction was repeated with the same primers under the same condition as described above to re-amplify the H151 cDNA, except that [α-³⁵S]-labeled dATP (1200 Ci/mmole) and 20 μM dNTP were not used herein.

2-13. TRG16

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 51 and a primer H-AP13 (5′-AAGCTTCGGCATA-3′) having a DNA sequence set forth in SEQ ID NO: 52 were used herein.

A 227-base pair (bp) band with H132 cDNA (Base positions from 813 to 1,039 of SEQ ID NO: 49) was cut out from the dried gel. The extracted gel was heated for 15 minutes to elute the H132 cDNA, and then the PCR reaction was repeated with the same primers under the same condition as described above to re-amplify the H132 cDNA, except that [α-³⁵S]-labeled dATP (1200 Ci/mmole) and 20 μM dNTP were not used herein.

2-14. TRG17

The PCR reaction was repeated in the same manner as in Example 2-1, except that an anchored primer H-T11G (5′-AAGCTTTTTTTTTTTG-3′, RNAimage kit, Genhunter, Cor., MA, U.S.) having a DNA sequence set forth in SEQ ID NO: 55 and a primer H-AP14 (5′-AAGCTTGGAGCTT-3′) having a DNA sequence set forth in SEQ ID NO: 56 were used herein.

A 185-base pair (bp) band with H141 cDNA (Base positions from 235 to 419 of SEQ ID NO: 53) was cut out from the dried gel. The extracted gel was heated for 15 minutes to elute the H141 cDNA, and then the PCR reaction was repeated with the same primers under the same condition as described above to re-amplify the H141 cDNA, except that [α-³⁵S]-labeled dATP (1200 Ci/mmole) and 20 μM dNTP were not used herein.

2-15. TRG18

The PCR reaction was repeated in the same manner as in Example 2-1, except that an anchored primer H-T11C (5′-AAGCTTTTTTTTTTTC-3′, RNAimage kit, Genhunter, Cor., MA, U.S.) having a DNA sequence set forth in SEQ ID NO: 59 and a primer H-AP18 (5′-AAGCTTAGAGGCA-3′) having a DNA sequence set forth in SEQ ID NO: 60 were used herein.

A 227-base pair (bp) band with H181 cDNA (Base positions from 902 to 1,128 of SEQ ID NO: 57) was cut out from the dried gel. The extracted gel was heated for 15 minutes to elute the H181 cDNA, and then the PCR reaction was repeated with the same primers under the same condition as described above to re-amplify the H181 cDNA, except that [α-³⁵S]-labeled dATP (1200 Ci/mmole) and 20 μM dNTP were not used herein.

2-16. TRG20

The PCR reaction was repeated in the same manner as in Example 2-1, except that an anchored primer H-T11A (5′-AAGCTTTTTTTTTTTA-3′, RNAimage kit, Genhunter, Cor., MA, U.S.) having a DNA sequence set forth in SEQ ID NO: 63 and a primer H-AP13 (5′-AAGCTTCGGCATA-3′) having a DNA sequence set forth in SEQ ID NO: 64 were used herein.

A 186-base pair (bp) band with H134 cDNA (Base positions from 232 to 417 of SEQ ID NO: 61) was cut out from the dried gel. The extracted gel was heated for 15 minutes to elute the H134 cDNA, and then the PCR reaction was repeated with the same primers under the same condition as described above to re-amplify the H134 cDNA, except that [α-³⁵S]-labeled dATP (1200 Ci/mmole) and 20 μM dNTP were not used herein.

### Example 3

### Cloning

The H20-121 product; the H93-811 product; the H117-321 product; the H38-211 product; the H38-621 product; the H96 product; the H94 product; the H42 product; the H109 product; the H119 product; the H201 product; the H151 product; the H132 product; the H141 product; the H181 product; and the H134 PCR product, which were all re-amplified as described above, were inserted into a pGEM-T EASY vector, respectively, according to the manufacturer's manual using the TA cloning system (Promega, U.S.).

(Step 1) Ligation Reaction

2 μl of each of the H20-121 product; the H93-811 product; the H117-321 product; the H38-211 product; the H38-621 product; the H96 product; the H94 product; the H42 product; the H109 product; the H119 product; the H201 product; the H151 product; the H132 product; the H141 product; the H181 product; and the H134 PCR product which were all re-amplified in Example 2, 1 μl of pGEM-T EASY vector (50 ng), 1 μl of T4 DNA ligase buffer (10×) and 1, of T4 DNA ligase (3 weiss units/μl; Promega) were put into a 0.5 μl test tube, and distilled water was added thereto to a final volume of 10 μl. The ligation reaction mixtures were incubated overnight at 14° C.

(Step 2) Transformation of TA Clone

E. coli JM109 (Promega, Wis., U.S.) was incubated in 10 mL of LB broth (10 g of bacto-tryptone, 5 g of bacto-yeast extract, 5 g of NaCl) until the optical density at 600 nm reached approximately 0.3 to 0.6. The incubated mixture was kept in ice for about 10 minutes, and then at 4° C. for 10 minutes, and centrifuged at 4,000 rpm for 10 minutes at 4° C., and then the supernatant wad discarded and the cell was collected. The collected cell pellet was exposed to 10 μml of 0.1 M ice-cold CaCl₂ for approximately 30 minutes to 1 hours to produce a competent cell. The resultant product was centrifuged again at 4,000 rpm for 10 minutes at 4° C., and then the supernatant wad discarded and the cell was collected and suspended in 2 ml of 0.1 M ice-cold CaCl₂.

200 μl of the competent cell suspension was transferred to a new microfuge tube, and 2 μl of each of the ligation reaction products prepared in Step 1 was added thereto. The resultant mixtures were incubated in a water bath at 42° C. for 90 seconds, and then quenched at 0° C. 800 μl of SOC medium (2.0 g of bacto-tryptone, 0.5 g of bacto-yeast extract, 1 μml of 1 M NaCl, 0.25 μml of 1 M KCl, 97 ml of TDW, 1 ml of 2 M Mg²⁺, 1 μml of 2 M glucose) was added thereto and the resultant mixtures were incubated at 37° C. for 45 minutes in a rotary shaking incubator at 220 rpm.

25 μl of X-gal (stored in 40 mg/ml of dimethylformamide) was spread with a glass rod on LB plates which were supplemented with ampicillin and previously maintained in the incubator at 37° C., and then 25 μl of each of the transformed cells was added thereto and spread again with a glass rod, and then incubated overnight at 37° C. After incubation, the 3 to 4 formed white colonies was selected and then each of the selected cells was seed-cultured in a LB plate which was supplemented with ampicillin. In order to construct plasmids, the strains proved to be colonies into which the ligation reaction products were introduced amongst the above colonies respectively, namely the transformed E. coli strains JM109/H20-121; JM109/H93-811; JM109/H117-321; JM109/H38-211; JM109/H38-621; JM109/H96; JM109/H94; JM109/H42; JM109/H109; JM109/H119; JM109/H201; JM109/H151; JM109/H132; JM109/H141; JM109/H181; and JM109/H134 were selected and incubated in 10 ml of terrific broth (900 ml of TDW, 12 g of bacto-tryptone, 24 g of bacto-yeast extract, 4 μml of glycerol, 0.17 M KH₂PO₄, 100 ml of 0.72 N K₂HPO₄).

### Example 4

### Separation of Recombinant Plasmid DNA

Each of the plasmid DNAs H20-121; H93-811; H117-321; H38-211; H38-621; H96; H94; H42; H109; H119; H201; H151; H132; H141; H181; and H134 was separated from the transformed E. coli strains using a Wizard™ Plus Minipreps DNA purification kit (Promega, U.S.) according to the manufacturer's manual according to the manufacturer's manual.

It was confirmed that a small amount of each of the separated plasmid DNAs was treated with a restriction enzyme ECoRI, and then electrophoresized in a 2% gel to confirm that the partial sequences of H20-121; H93-811; H117-321; H38-211; H38-621; H96; H94; H42; H109; H119; H201; H151; H132; H141; H181; and H134 were inserted into the plasmids, respectively.

### Example 5

### DNA Base Sequence Analysis

5-1. TRG3

The H20-121 PCR product obtained in Example 2 was amplified, cloned, and then re-amplified according to the conventional method. The resultant H20-121 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1,342 to 1,599 of SEQ ID NO: 1, which was designated “H20-121” in the present invention.

The 258-bp cDNA fragment obtained above, namely H20-121, was subject to the differential display reverse transcription-polymerase chain reaction (DDRT-PCR) using a 5′-random primer H-AP20 and a 3′-anchored primer H-T11A, and then confirmed using the electrophoresis.

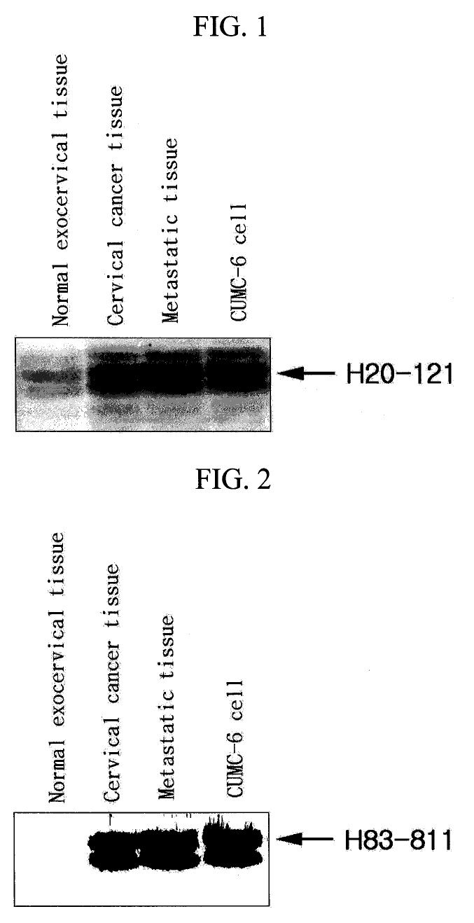

As shown in FIG. 1, it was revealed from the differential display (DD) that the gene was differentially expressed in the normal exocervical tissue, the metastatic lymph node tissue and the CUMC-6 cell. As seen in FIG. 1, the 258-bp cDNA fragment H20-121 was expressed in the cervical cancer, the metastatic lymph node tissue and the CUMC-6 cancer cell, but very rarely expressed in the normal tissue.

5-2. TRG4

The H93-811 PCR product obtained in Example 2 was amplified, cloned, and then re-amplified according to the conventional method. The resultant H93-811 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 2,086 to 2,478 of SEQ ID NO: 5, which was designated “H93-811” in the present invention.

The 393-bp cDNA fragment obtained above, namely H93-811, was subject to the differential display reverse transcription-polymerase chain reaction (DDRT-PCR) using a 5′-random primer H-AP9 and a 3′-anchored primer H-T11G, and then confirmed using the electrophoresis.

As shown in FIG. 2, it was revealed from the differential display (DD) that the gene was differentially expressed in the normal exocervical tissue, the metastatic lymph node tissue and the CUMC-6 cell. As seen in FIG. 1, the 393-bp cDNA fragment H93-811 was expressed in the cervical cancer, the metastatic lymph node tissue and the CUMC-6 cancer cell, but very rarely expressed in the normal tissue.

5-3. TRG5

The H117-321 PCR product obtained in Example 2 was amplified, cloned, and then re-amplified according to the conventional method. The resultant H117-321 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 933 to 1,224 of SEQ ID NO: 9, which was designated “H117-321” in the present invention.

The 292-bp cDNA fragment obtained above, namely H117-321, was subject to the differential display reverse transcription-polymerase chain reaction (DDRT-PCR) using a 5′-random primer H-AP11 and a 3′-anchored primer H-T11C, and then confirmed using the electrophoresis.

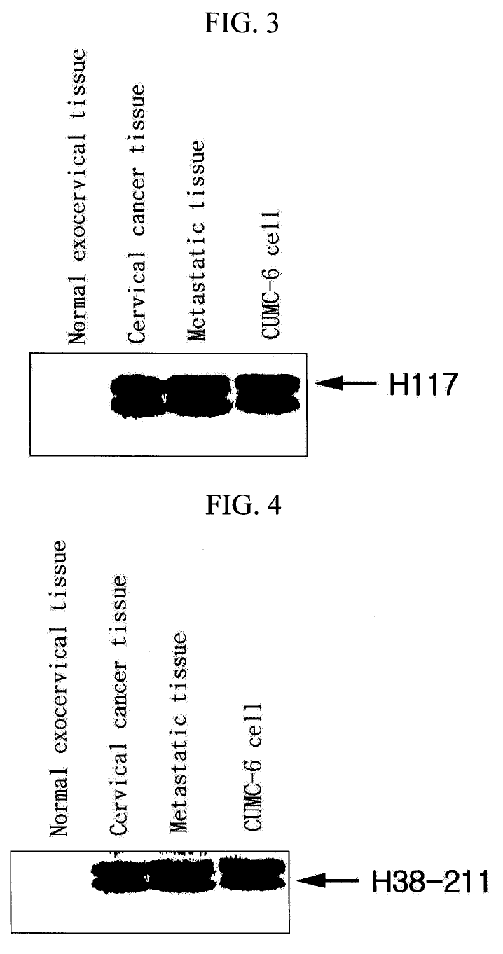

As shown in FIG. 3, it was revealed from the differential display (DD) that the gene was differentially expressed in the normal exocervical tissue, the metastatic lymph node tissue and the CUMC-6 cell. As seen in FIG. 3, the 292-bp cDNA fragment H117-321 was expressed in the cervical cancer, the metastatic lymph node tissue and the CUMC-6 cancer cell, but very rarely expressed in the normal tissue.

5-4. TRG6

The H38-211 PCR product obtained in Example 2 was amplified, cloned, and then re-amplified according to the conventional method. The resultant H38-211 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 2,823 to 3,133 of SEQ ID NO: 13, which was designated “H38-211” in the present invention. The 311-bp cDNA fragment obtained above, namely H38-211, was subject to the differential display reverse transcription-polymerase chain reaction (DDRT-PCR) using a 5′-random primer H-AP38 and a 3′-anchored primer H-T11A, and then confirmed using the electrophoresis.

As shown in FIG. 4, it was revealed from the differential display (DD) that the gene was differentially expressed in the normal exocervical tissue, the metastatic lymph node tissue and the CUMC-6 cell. As seen in FIG. 4, the 311-bp cDNA fragment H38-211 was expressed in the cervical cancer, the metastatic lymph node tissue and the CUMC-6 cancer cell, but very rarely expressed in the normal tissue.

5-5. TRG7

The H38-621 PCR product obtained in Example 2 was amplified, cloned, and then re-amplified according to the conventional method. The resultant H38-621 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1,404 to 1,695 of SEQ ID NO: 17, which was designated “H38-621” in the present invention.

The 292-bp cDNA fragment obtained above, namely H38-621, was subject to the differential display reverse transcription-polymerase chain reaction (DDRT-PCR) using a 5′-random primer H-AP38 and a 3′-anchored primer H-T11G, and then confirmed using the electrophoresis.

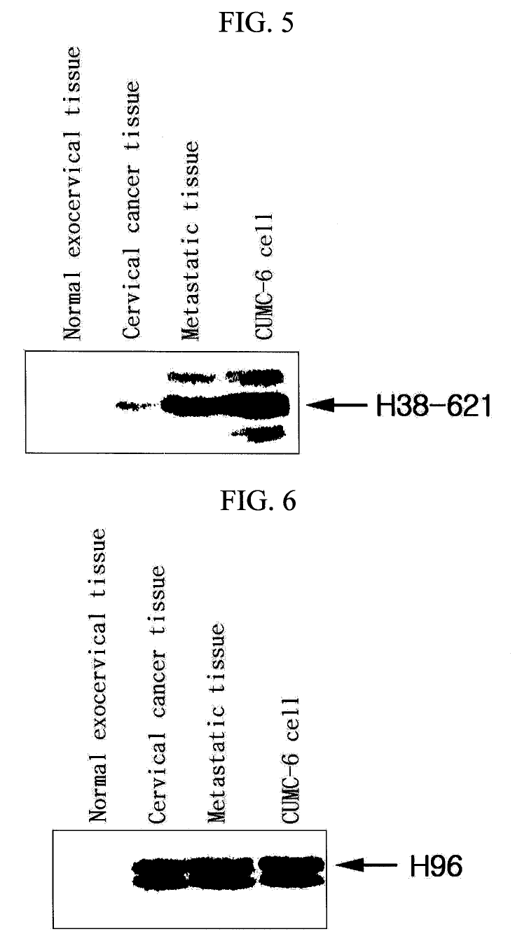

As shown in FIG. 5, it was revealed from the differential display (DD) that the gene was differentially expressed in the normal exocervical tissue, the metastatic lymph node tissue and the CUMC-6 cell. As seen in FIG. 5, the 292-bp cDNA fragment H38-621 was expressed in the cervical cancer, the metastatic lymph node tissue and the CUMC-6 cancer cell, but very rarely expressed in the normal tissue.

5-6. TRG9

The H96 PCR product obtained in Example 2 was amplified, cloned, and then re-amplified according to the conventional method. The resultant H96 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1,225 to 1,499 of SEQ ID NO: 21, which was designated “H96” in the present invention.

The 275-bp cDNA fragment obtained above, namely H96, was subject to the differential display reverse transcription-polymerase chain reaction (DDRT-PCR) using a 5′-random primer H-AP9 and a 3′-anchored primer H-T11A, and then confirmed using the electrophoresis.

As shown in FIG. 6, it was revealed from the differential display (DD) that the gene was differentially expressed in the normal exocervical tissue, the metastatic lymph node tissue and the CUMC-6 cell. As seen in FIG. 6, the 275-bp cDNA fragment H96 was expressed in the cervical cancer, the metastatic lymph node tissue and the CUMC-6 cancer cell, but very rarely expressed in the normal tissue.

5-7. TRG10

The H94 PCR product obtained in Example 2 was amplified, cloned, and then re-amplified according to the conventional method. The resultant H94 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 3,528 to 3,879 of SEQ ID NO: 25, which was designated “H94” in the present invention.

The 352-bp cDNA fragment obtained above, namely H94, was subject to the differential display reverse transcription-polymerase chain reaction (DDRT-PCR) using a 5′-random primer H-AP9 and a 3′-anchored primer H-T11A, and then confirmed using the electrophoresis.

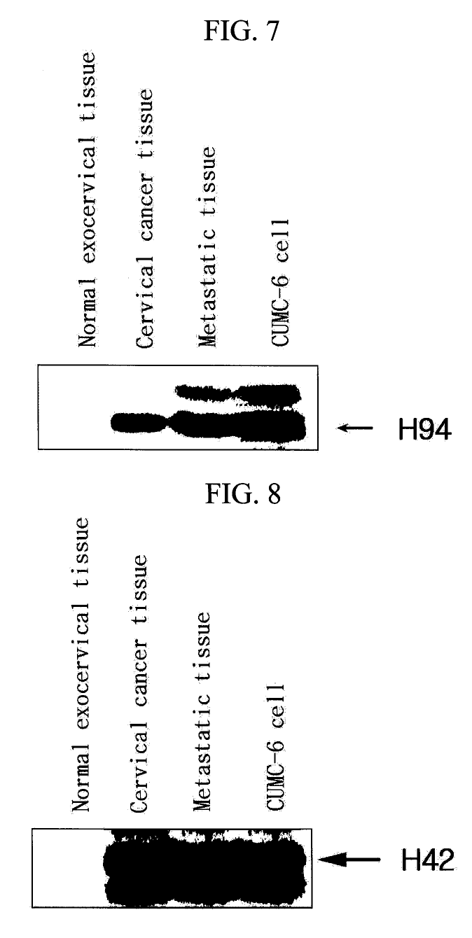

As shown in FIG. 7, it was revealed from the differential display (DD) that the gene was differentially expressed in the normal exocervical tissue, the metastatic lymph node tissue and the CUMC-6 cell. As seen in FIG. 7, the 352-bp cDNA fragment H94 was expressed in the cervical cancer, the metastatic lymph node tissue and the CUMC-6 cancer cell, but rarely expressed in the normal tissue.

5-8. TRG11

The H42 PCR product obtained in Example 2 was amplified, cloned, and then re-amplified according to the conventional method. The resultant H42 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 83 to 229 of SEQ ID NO: 29, which was designated “H42” in the present invention.

The 147-bp cDNA fragment obtained above, namely H42, was subject to the differential display reverse transcription-polymerase chain reaction (DDRT-PCR) using a 5′-random primer H-AP4 and a 3′-anchored primer H-T11C, and then confirmed using the electrophoresis.

As shown in FIG. 8, it was revealed from the differential display (DD) that the gene was differentially expressed in the normal exocervical tissue, the metastatic lymph node tissue and the CUMC-6 cell. As seen in FIG. 8, the 147-bp cDNA fragment H42 was expressed in the cervical cancer, the metastatic lymph node tissue and the CUMC-6 cancer cell, but rarely expressed in the normal tissue.

5-9. TRG12

The H109 PCR product obtained in Example 2 was amplified, cloned, and then re-amplified according to the conventional method. The resultant H109 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 284 to 495 of SEQ ID NO: 33, which was designated “H109” in the present invention.

The 212-bp cDNA fragment obtained above, namely H109, was subject to the differential display reverse transcription-polymerase chain reaction (DDRT-PCR) using a 5′-random primer H-AP10 and a 3′-anchored primer H-T11G, and then confirmed using the electrophoresis.

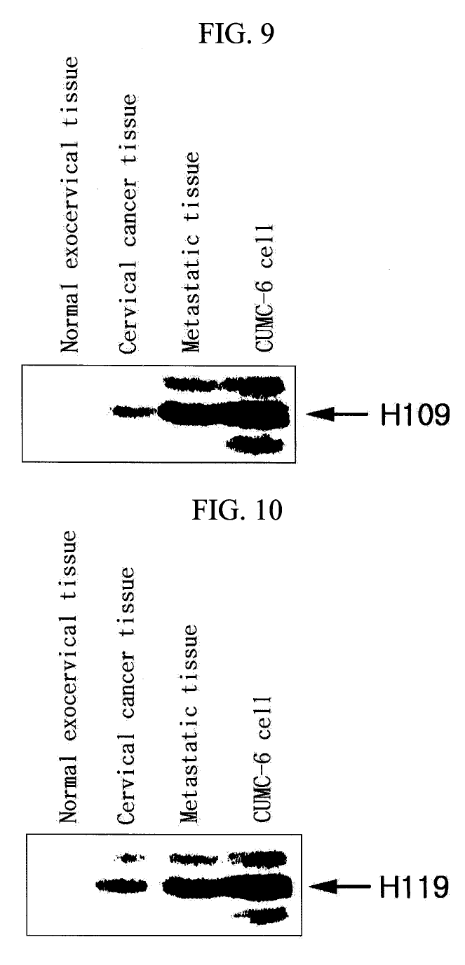

As shown in FIG. 9, it was revealed from the differential display (DD) that the gene was differentially expressed in the normal exocervical tissue, the metastatic lymph node tissue and the CUMC-6 cell. As seen in FIG. 9, the 212-bp cDNA fragment H109 was expressed in the cervical cancer, the metastatic lymph node tissue and the CUMC-6 cancer cell, but very rarely expressed in the normal tissue.

5-10. TRG13

The H119 PCR product obtained in Example 2 was amplified, cloned, and then re-amplified according to the conventional method. The resultant H119 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1,004 to 1,235 of SEQ ID NO: 37, which was designated “H119” in the present invention.

The 232-bp cDNA fragment obtained above, namely H119, was subject to the differential display reverse transcription-polymerase chain reaction (DDRT-PCR) using a 5′-random primer H-AP11 and a 3′-anchored primer H-T11C, and then confirmed using the electrophoresis.

As shown in FIG. 10, it was revealed from the differential display (DD) that the gene was differentially expressed in the normal exocervical tissue, the metastatic lymph node tissue and the CUMC-6 cell. As seen in FIG. 10, the 232-bp cDNA fragment H119 was expressed in the cervical cancer, the metastatic lymph node tissue and the CUMC-6 cancer cell, but very rarely expressed in the normal tissue.

5-11. TRG14

The H201 PCR product obtained in Example 2 was amplified, cloned, and then re-amplified according to the conventional method. The resultant H201 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 902 to 1,096 of SEQ ID NO: 41, which was designated “H201” in the present invention.

The 195-bp cDNA fragment obtained above, namely H201, was subject to the differential display reverse transcription-polymerase chain reaction (DDRT-PCR) using a 5′-random primer H-AP20 and a 3′-anchored primer H-T11G, and then confirmed using the electrophoresis.

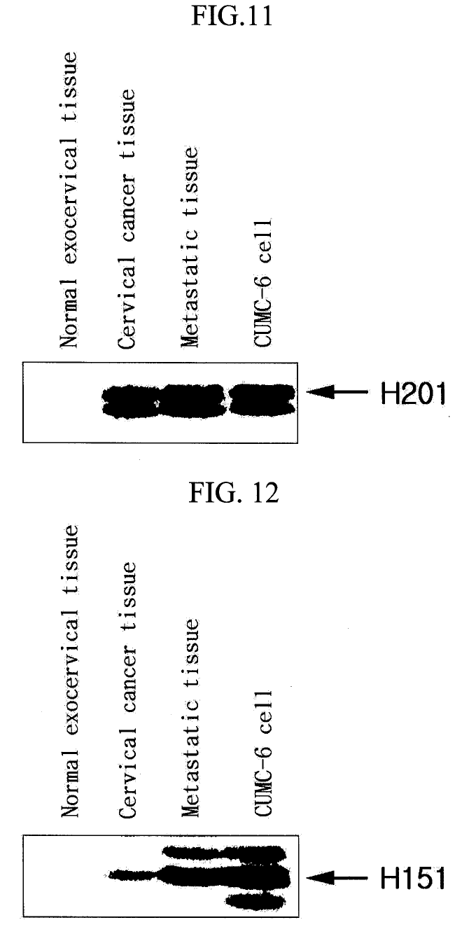

As shown in FIG. 11, it was revealed from the differential display (DD) that the gene was differentially expressed in the normal exocervical tissue, the metastatic lymph node tissue and the CUMC-6 cell. As seen in FIG. 11, the 195-bp cDNA fragment H201 was expressed in the cervical cancer, the metastatic lymph node tissue and the CUMC-6 cancer cell, but very rarely expressed in the normal tissue.

5-12. TRG15

The H151 PCR product obtained in Example 2 was amplified, cloned, and then re-amplified according to the conventional method. The resultant H151 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 848 to 1,099 of SEQ ID NO: 45, which was designated “H151” in the present invention.

The 252-bp cDNA fragment obtained above, namely H151, was subject to the differential display reverse transcription-polymerase chain reaction (DDRT-PCR) using a 5′-random primer H-AP15 and a 3′-anchored primer H-T11A, and then confirmed using the electrophoresis.

As shown in FIG. 12, it was revealed from the differential display (DD) that the gene was differentially expressed in the normal exocervical tissue, the metastatic lymph node tissue and the CUMC-6 cell. As seen in FIG. 12, the 252-bp cDNA fragment H151 was expressed in the cervical cancer, the metastatic lymph node tissue and the CUMC-6 cancer cell, but very rarely expressed in the normal tissue.

5-13. TRG16

The H132 PCR product obtained in Example 2 was amplified, cloned, and then re-amplified according to the conventional method. The resultant H132 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 813 to 1,039 of SEQ ID NO: 49, which was designated “H132” in the present invention.

The 227-bp cDNA fragment obtained above, namely H132, was subject to the differential display reverse transcription-polymerase chain reaction (DDRT-PCR) using a 5′-random primer H-AP13 and a 3′-anchored primer H-T11C, and then confirmed using the electrophoresis.

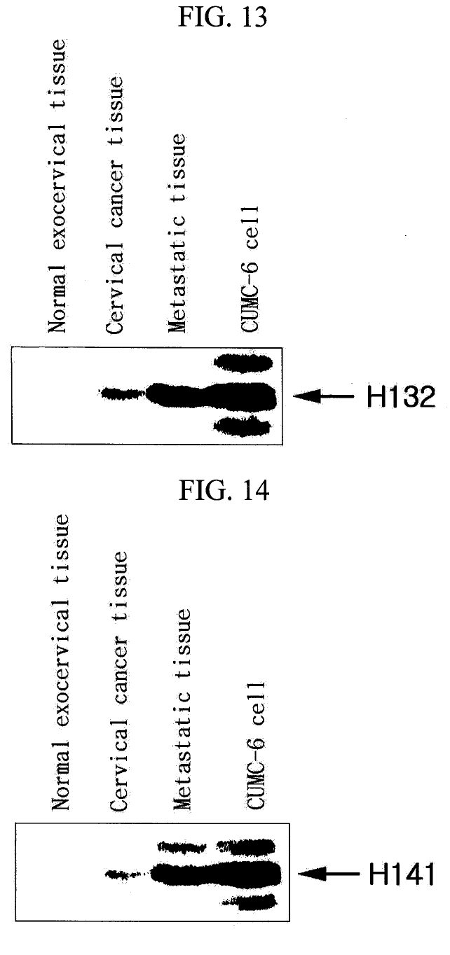

As shown in FIG. 13, it was revealed from the differential display (DD) that the gene was differentially expressed in the normal exocervical tissue, the metastatic lymph node tissue and the CUMC-6 cell. As seen in FIG. 13, the 227-bp cDNA fragment H132 was expressed in the cervical cancer, the metastatic lymph node tissue and the CUMC-6 cancer cell, but very rarely expressed in the normal tissue.

5-14. TRG17

The H141 PCR product obtained in Example 2 was amplified, cloned, and then re-amplified according to the conventional method. The resultant H141 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 235 to 419 of SEQ ID NO: 53, which was designated “H141” in the present invention.

The 185-bp cDNA fragment obtained above, namely H141, was subject to the differential display reverse transcription-polymerase chain reaction (DDRT-PCR) using a 5′-random primer H-AP14 and a 3′-anchored primer H-T11G, and then confirmed using the electrophoresis.

As shown in FIG. 14, it was revealed from the differential display (DD) that the gene was differentially expressed in the normal exocervical tissue, the metastatic lymph node tissue and the CUMC-6 cell. As seen in FIG. 14, the 185-bp cDNA fragment H141 was expressed in the cervical cancer, the metastatic lymph node tissue and the CUMC-6 cancer cell, but very rarely expressed in the normal tissue.

5-15. TRG18

The H181 PCR product obtained in Example 2 was amplified, cloned, and then re-amplified according to the conventional method. The resultant H181 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 902 to 1,128 of SEQ ID NO: 57, which was designated “H181” in the present invention.

The 227-bp cDNA fragment obtained above, namely H181, was subject to the differential display reverse transcription-polymerase chain reaction (DDRT-PCR) using a 5′-random primer H-AP18 and a 3′-anchored primer H-T11C, and then confirmed using the electrophoresis.

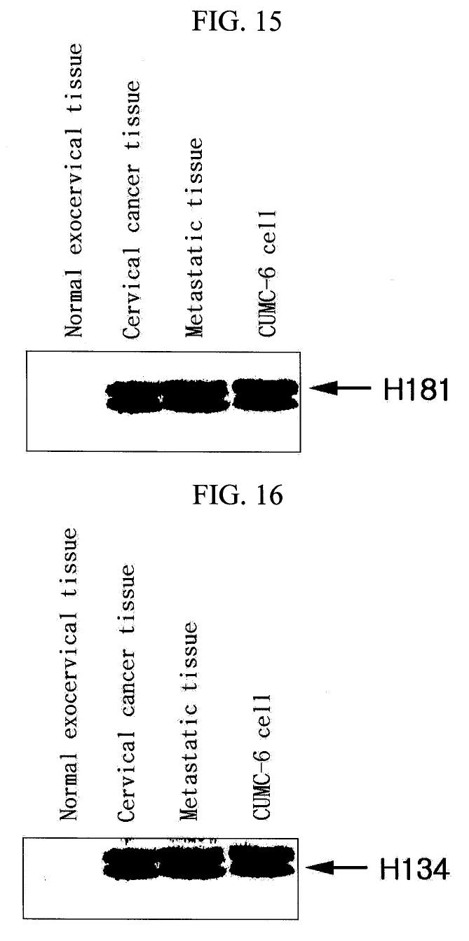

As shown in FIG. 15, it was revealed from the differential display (DD) that the gene was differentially expressed in the normal exocervical tissue, the metastatic lymph node tissue and the CUMC-6 cell. As seen in FIG. 15, the 227-bp cDNA fragment H181 was expressed in the cervical cancer, the metastatic lymph node tissue and the CUMC-6 cancer cell, but very rarely expressed in the normal tissue.

5-16. TRG20

The H134 PCR product obtained in Example 2 was amplified, cloned, and then re-amplified according to the conventional method. The resultant H134 PCT fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 232 to 417 of SEQ ID NO: 61, which was designated “H134” in the present invention.

The 186-bp cDNA fragment obtained above, namely H134, was subject to the differential display reverse transcription-polymerase chain reaction (DDRT-PCR) using a 5′-random primer H-AP13 and a 3′-anchored primer H-T11A, and then confirmed using the electrophoresis.

As shown in FIG. 16, it was revealed from the differential display (DD) that the gene was differentially expressed in the normal exocervical tissue, the metastatic lymph node tissue and the CUMC-6 cell. As seen in FIG. 16, the 186-bp cDNA fragment H134 was expressed in the cervical cancer, the metastatic lymph node tissue and the CUMC-6 cancer cell, but very rarely expressed in the normal tissue.

### Example 6

### cDNA Sequence Analysis of Full-Length TRG Protooncogene

6-1. TRG3

The ³²P-labeled H20-121 was used as the probe to screen a bacteriophage λgt11 human lung embryonic fibroblast cDNA library (Miki, T. et al., Gene 83: 137-146, 1989). A full-length TRG3 cDNA clone, in which the 1,703-bp fragment was inserted into the pCEV-LAC vector, was obtained from the human lung embryonic fibroblast cDNA library, and then deposited with Accession No. AY189688 in the U.S. GenBank database on Dec. 2, 2002 (Scheduled Release Date: Apr. 8, 2005).

The TRG3 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989). The pCEV-LAC vector containing the TRG3 gene was ligated by T4 DNA ligase to prepare TRG3 plasmid DNA, and then E. coli DH5α was transformed with the ligated clone.

The full-length DNA sequence of the TRG3 consisting of 1,703 bp was set forth in SEQ ID NO: 1.

In the DNA sequence of SEQ ID NO: 1, it is estimated that a full-length open reading frame of the protooncogene of the present invention corresponds to nucleotide sequence positions from 113 to 1,522, and encodes a protein consisting of 469 amino acids of SEQ ID NO: 2.

6-2. TRG4

The ³²P-labeled H93-811 was used as the probe to screen a bacteriophage λgt11 human lung embryonic fibroblast cDNA library (Miki, T. et al., Gene 83: 137-146, 1989). A full-length TRG4 cDNA clone, in which the 2,576-bp fragment was inserted into the pCEV-LAC vector, was obtained from the human lung embryonic fibroblast cDNA library, and then deposited with Accession No. AY189690 in the U.S. GenBank database on Dec. 2, 2002 (Scheduled Release Date: Apr. 8, 2005). The TRG4 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989). The pCEV-LAC vector containing the TRG4 gene was ligated by T4 DNA ligase to prepare TRG4 plasmid DNA, and then E. coli DH5α was transformed with the ligated clone. The full-length DNA sequence of the TRG4 consisting of 2,576 bp was set forth in SEQ ID NO: 5. In the DNA sequence of SEQ ID NO: 5, it is estimated that a full-length open reading frame of the protooncogene of the present invention corresponds to nucleotide sequence positions from 87 to 482, and encodes a protein consisting of 131 amino acids of SEQ ID NO: 6.

6-3. TRG5

The ³²P-labeled H17-321 was used as the probe to screen a bacteriophage λgt11 human lung embryonic fibroblast cDNA library (Miki, T. et al., Gene 83: 137-146, 1989). A full-length TRG5 cDNA clone, in which the 1,334-bp fragment was inserted into the pCEV-LAC vector, was obtained from the human lung embryonic fibroblast cDNA library, and then deposited with Accession No. AY189689 in the U.S. GenBank database on Dec. 2, 2002 (Scheduled Release Date: Apr. 8, 2005).

The TRG5 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989). The pCEV-LAC vector containing the TRG5 gene was ligated by T4 DNA ligase to prepare TRG5 plasmid DNA, and then E. coli DH5α was transformed with the ligated clone. The full-length DNA sequence of the TRG5 consisting of 1,336 bp was set forth in SEQ ID NO: 9.

In the DNA sequence of SEQ ID NO: 9, it is estimated that a full-length open reading frame of the protooncogene of the present invention corresponds to nucleotide sequence positions from 88 to 1,092, and encodes a protein consisting of 334 amino acids of SEQ ID NO: 10.

6-4. TRG6

The ³²P-labeled H38-211 was used as the probe to screen a bacteriophage λgt11 human lung embryonic fibroblast cDNA library (Miki, T. et al., Gene 83: 137-146, 1989). A full-length TRG6 cDNA clone, in which the 3,309-bp fragment was inserted into the pCEV-LAC vector, was obtained from the human lung embryonic fibroblast cDNA library, and then deposited with Accession No. AY191222 in the U.S. GenBank database on Dec. 5, 2002 (Scheduled Release Date: Apr. 8, 2005). The TRG6 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG6 gene was ligated by T4 DNA ligase to prepare TRG6 plasmid DNA, and then E. coli DH5 CL was transformed with the ligated clone.

The full-length DNA sequence of the TRG6 consisting of 3,309 bp was set forth in SEQ ID NO: 13.

In the DNA sequence of SEQ ID NO: 13, it is estimated that a full-length open reading frame of the protooncogene of the present invention corresponds to nucleotide sequence positions from 233 to 481, and encodes a protein consisting of 82 amino acids of SEQ ID NO: 14.

6-5. TRG7

The ³²P-labeled H38-621 was used as the probe to screen a bacteriophage λgt11 human lung embryonic fibroblast cDNA library (Miki, T. et al., Gene 83: 137-146, 1989). A full-length TRG7 cDNA clone, in which the 1,778-bp fragment was inserted into the pCEV-LAC vector, was obtained from the human lung embryonic fibroblast cDNA library, and then deposited with Accession No. AY191223 in the U.S. GenBank database on Dec. 5, 2002 (Scheduled Release Date: Apr. 8, 2005). The TRG7 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG7 gene was ligated by T4 DNA ligase to prepare TRG7 plasmid DNA, and then E. coli DH5 CL was transformed with the ligated clone. The full-length DNA sequence of the TRG7 consisting of 1,778 bp was set forth in SEQ ID NO: 17.

In the DNA sequence of SEQ ID NO: 17, it is estimated that a full-length open reading frame of the protooncogene of the present invention corresponds to nucleotide sequence positions from 42 to 1,422, and encodes a protein consisting of 175 amino acids of SEQ ID NO: 18.

6-6. TRG9

The ³²P-labeled H96 was used as the probe to screen a bacteriophage λgt11 human lung embryonic fibroblast cDNA library (Miki, T. et al., Gene 83: 137-146, 1989). A full-length TRG9 cDNA clone, in which the 1,582-bp fragment was inserted into the pCEV-LAC vector, was obtained from the human lung embryonic fibroblast cDNA library, and then deposited with Accession No. AY272044 in the U.S. GenBank database on Apr. 9, 2003 (Scheduled Release Date: Mar. 31, 2005).

The TRG9 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989). The pCEV-LAC vector containing the TRG9 gene was ligated by T4 DNA ligase to prepare TRG9 plasmid DNA, and then E. coli DH5α was transformed with the ligated clone.

The full-length DNA sequence of the TRG9 consisting of 1,582 bp was set forth in SEQ ID NO: 21.

In the DNA sequence of SEQ ID NO: 21, it is estimated that a full-length open reading frame of the protooncogene of the present invention corresponds to nucleotide sequence positions from 17 to 1,576, and encodes a protein consisting of 519 amino acids of SEQ ID NO: 22.

6-7. TRG10

The ³²P-labeled H94 was used as the probe to screen a bacteriophage λgt11 human lung embryonic fibroblast cDNA library (Miki, T. et al., Gene 83: 137-146, 1989). A full-length TRG10 cDNA clone, in which the 3,979-bp fragment was inserted into the pCEV-LAC vector, was obtained from the human lung embryonic fibroblast cDNA library, and then deposited with Accession No. AY277593 in the U.S. GenBank database on Apr. 12, 2003 (Scheduled Release Date: Mar. 31, 2005).

The TRG10 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG10 gene was ligated by T4 DNA ligase to prepare TRG10 plasmid DNA, and then E. coli DH5α was transformed with the ligated clone.

The full-length DNA sequence of the TRG10 consisting of 3,979 bp was set forth in SEQ ID NO: 25.

In the DNA sequence of SEQ ID NO: 25, it is estimated that a full-length open reading frame of the protooncogene of the present invention corresponds to nucleotide sequence positions from 1,100 to 1,270, and encodes a protein consisting of 56 amino acids of SEQ ID NO: 26.

6-8. TRG11

The ³²P-labeled H42 was used as the probe to screen a bacteriophage λgt11 human lung embryonic fibroblast cDNA library (Miki, T. et al., Gene 83: 137-146, 1989). A full-length TRG11 cDNA clone, in which the 235-bp fragment was inserted into the pCEV-LAC vector, was obtained from the human lung embryonic fibroblast cDNA library, and then deposited with Accession No. AY277594 in the U.S. GenBank database on Apr. 13, 2003 (Scheduled Release Date: Mar. 31, 2005).

The TRG11 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG11 gene was ligated by T4 DNA ligase to prepare TRG11 plasmid DNA, and then E. coli DH5α was transformed with the ligated clone.

The full-length DNA sequence of the TRG11 consisting of 235 bp was set forth in SEQ ID NO: 29.

In the DNA sequence of SEQ ID NO: 29, it is estimated that a full-length open reading frame of the protooncogene of the present invention corresponds to nucleotide sequence positions from 26 to 214, and encodes a protein consisting of 62 amino acids of SEQ ID NO: 30.

6-9. TRG12

The ³²P-labeled H109 was used as the probe to screen a bacteriophage λgt11 human lung embryonic fibroblast cDNA library (Miki, T. et al., Gene 83: 137-146, 1989). A full-length TRG12 cDNA clone, in which the 510-bp fragment was inserted into the pCEV-LAC vector, was obtained from the human lung embryonic fibroblast cDNA library, and then deposited with Accession No. AY277595 in the U.S. GenBank database on Apr. 13, 2003 (Scheduled Release Date: Mar. 31, 2005).

The TRG12 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG12 gene was ligated by T4 DNA ligase to prepare TRG12 plasmid DNA, and then E. coli DH5α was transformed with the ligated clone.

The full-length DNA sequence of the TRG12 consisting of 510 bp was set forth in SEQ ID NO: 33.

In the DNA sequence of SEQ ID NO: 33, it is estimated that a full-length open reading frame of the protooncogene of the present invention corresponds to nucleotide sequence positions from 80 to 475, and encodes a protein consisting of 131 amino acids of SEQ ID NO: 34.

6-10. TRG13

The ³²P-labeled H119 was used as the probe to screen a bacteriophage λgt11 human lung embryonic fibroblast cDNA library (Miki, T. et al., Gene 83: 137-146, 1989). A full-length TRG13 cDNA clone, in which the 1,301-bp fragment was inserted into the pCEV-LAC vector, was obtained from the human lung embryonic fibroblast cDNA library, and then deposited with Accession No. AY277596 in the U.S. GenBank database on Apr. 13, 2003 (Scheduled Release Date: Mar. 31, 2005).

The TRG13 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG13 gene was ligated by T4 DNA ligase to prepare TRG13 plasmid DNA, and then E. coli DH5α was transformed with the ligated clone.

The full-length DNA sequence of the TRG13 consisting of 1,301 bp was set forth in SEQ ID NO: 37.

In the DNA sequence of SEQ ID NO: 37, it is estimated that a full-length open reading frame of the protooncogene of the present invention corresponds to nucleotide sequence positions from 18 to 1,193, and encodes a protein consisting of 391 amino acids of SEQ ID NO: 38.

6-11. TRG14

The ³²P-labeled H201 was used as the probe to screen a bacteriophage λgt11 human lung embryonic fibroblast cDNA library (Miki, T. et al., Gene 83: 137-146, 1989). A full-length TRG14 cDNA clone, in which the 1,206-bp fragment was inserted into the pCEV-LAC vector, was obtained from the human lung embryonic fibroblast cDNA library, and then deposited with Accession No. AY277597 in the U.S. GenBank database on Apr. 13, 2003 (Scheduled Release Date: Mar. 31, 2005).

The TRG14 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG14 gene was ligated by T4 DNA ligase to prepare TRG14 plasmid DNA, and then E. coli DH5α was transformed with the ligated clone.

The full-length DNA sequence of the TRG14 consisting of 1,206 bp was set forth in SEQ ID NO: 41.

In the DNA sequence of SEQ ID NO: 41, it is estimated that a full-length open reading frame of the protooncogene of the present invention corresponds to nucleotide sequence positions from 18 to 1,202, and encodes a protein consisting of 394 amino acids of SEQ ID NO: 42.

6-12. TRG15

The ³²P-labeled H151 was used as the probe to screen a bacteriophage λgt11 human lung embryonic fibroblast cDNA library (Miki, T. et al., Gene 83: 137-146, 1989). A full-length TRG15 cDNA clone, in which the 1,104-bp fragment was inserted into the pCEV-LAC vector, was obtained from the human lung embryonic fibroblast cDNA library, and then deposited with Accession No. AY277598 in the U.S. GenBank database on Apr. 13, 2003 (Scheduled Release Date: Mar. 31, 2005).

The TRG15 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG15 gene was ligated by T4 DNA ligase to prepare TRG15 plasmid DNA, and then E. coli DH5α was transformed with the ligated clone.

The full-length DNA sequence of the TRG15 consisting of 1,104 bp was set forth in SEQ ID NO: 45.

In the DNA sequence of SEQ ID NO: 45, it is estimated that a full-length open reading frame of the protooncogene of the present invention corresponds to nucleotide sequence positions from 1 to 1,104, and encodes a protein consisting of 367 amino acids of SEQ ID NO: 46.

6-13. TRG16

The ³²P-labeled H132 was used as the probe to screen a bacteriophage λgt11 human lung embryonic fibroblast cDNA library (Miki, T. et al., Gene 83: 137-146, 1989). A full-length TRG16 cDNA clone, in which the 1,064-bp fragment was inserted into the pCEV-LAC vector, was obtained from the human lung embryonic fibroblast cDNA library, and then deposited with Accession No. AY277601 in the U.S. GenBank database on Apr. 14, 2003 (Scheduled Release Date: Mar. 31, 2005).

The TRG16 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG16 gene was ligated by T4 DNA ligase to prepare TRG16 plasmid DNA, and then E. coli DH5α was transformed with the ligated clone.

The full-length DNA sequence of the TRG16 consisting of 1,064 bp was set forth in SEQ ID NO: 49.

In the DNA sequence of SEQ ID NO: 49, it is estimated that a full-length open reading frame of the protooncogene of the present invention corresponds to nucleotide sequence positions from 92 to 1,064, and encodes a protein consisting of 324 amino acids of SEQ ID NO: 50.

6-14. TRG17

The ³²P-labeled H141 was used as the probe to screen a bacteriophage λgt11 human lung embryonic fibroblast cDNA library (Miki, T. et al., Gene 83: 137-146, 1989). A full-length TRG17 cDNA clone, in which the 432-bp fragment was inserted into the pCEV-LAC vector, was obtained from the human lung embryonic fibroblast cDNA library, and then deposited with Accession No. AY277599 in the U.S. GenBank database on Apr. 13, 2003 (Scheduled Release Date: Mar. 31, 2005). The TRG17 clone inserted into the λpCEV vector was cleaved by the restriction enzyme NotI and isolated from the phage in a form of ampicillin-resistant PCEV-LAC phagemid vector (Miki, T. et al., Gene 83: 137-146, 1989).

The pCEV-LAC vector containing the TRG17 gene was ligated by T4 DNA ligase to prepare TRG17 plasmid DNA, and then E. coli DH5α was transformed with the ligated clone.

The full-length DNA sequence of the TRG17 consisting of 432 bp was set forth in SEQ ID NO: 53.

In the DNA sequence of SEQ ID NO: 53, it is estimated that a full-length open reading frame of the protooncogene of the present invention corresponds to nucleotide sequence positions from 1 to 408, and encodes a protein consisting of 135 amino acids of SEQ ID NO: 54.

6-15. TRG18

The ³²P-labeled H181 was used as the probe to screen a bacteriophage λgt11 human lung embryonic fibroblast cDNA library (Miki, T. et al., Gene 83: 137-146, 1989). A full-length TRG18 cDNA clone, in which the 1,141-bp fragment was inserted into the pCEV-LAC vector, was obtained from the human lung embryonic fibroblast cDNA library, and then deposited with Accession No. AY277600 in the U.S. GenBank database on Apr. 13, 2003 (Scheduled Release Date: Mar. 31, 2005).

The TRG18 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG18 gene was ligated by T4 DNA ligase to prepare TRG18 plasmid DNA, and then E. coli DH5α was transformed with the ligated clone.

The full-length DNA sequence of the TRG18 consisting of 1,141 bp was set forth in SEQ ID NO: 57.

In the DNA sequence of SEQ ID NO: 57, it is estimated that a full-length open reading frame of the protooncogene of the present invention corresponds to nucleotide sequence positions from 20 to 1,141, and encodes a protein consisting of 373 amino acids of SEQ ID NO: 58.

6-16. TRG20

The ³²P-labeled H134 was used as the probe to screen a bacteriophage λgt11 human lung embryonic fibroblast cDNA library (Miki, T. et al., Gene 83: 137-146, 1989). A full-length TRG20 cDNA clone, in which the 449-bp fragment was inserted into the pCEV-LAC vector, was obtained from the human lung embryonic fibroblast cDNA library, and then deposited with Accession No. AY453397 in the U.S. GenBank database on Oct. 29, 2003 (Scheduled Release Date: Mar. 31, 2005).

The TRG20 clone inserted into the λpCEV vector was cleaved by the restriction enzyme NotI and isolated from the phage in a form of ampicillin-resistant pCEV-LAC phagemid vector (Miki, T. et al., Gene 83: 137-146, 1989).

The pCEV-LAC vector containing the TRG20 gene was ligated by T4 DNA ligase to prepare TRG20 plasmid DNA, and then E. coli DH5α was transformed with the ligated clone.

The full-length DNA sequence of the TRG20 consisting of 449 bp was set forth in SEQ ID NO: 61.

In the DNA sequence of SEQ ID NO: 61, it is estimated that a full-length open reading frame of the protooncogene of the present invention corresponds to nucleotide sequence positions from 42 to 449, and encodes a protein consisting of 135 amino acids of SEQ ID NO: 62.

### Example 7

### Northern Blotting Analysis of TRG Genes in Various Cells

The total RNA samples were extracted from the normal exocervical tissue, the cervical cancer tissue, the metastatic cervical lymph node tissue and the cervical cancer cell lines CaSki (ATCC CRL 1550) and CUMC-6 in the same manner as in Example 1.

In order to determine an expression level of each of the TRG genes, 20 μg of each of the total denatured RNA samples extracted from the tissues and cell lines was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed full-length TRG cDNA probe prepared using the Rediprime II random prime labelling system ((Amersham, United Kingdom). The northern blotting analysis was repeated twice, and then the resultant blots were quantified with the densitometer and normalized with the β-actin.

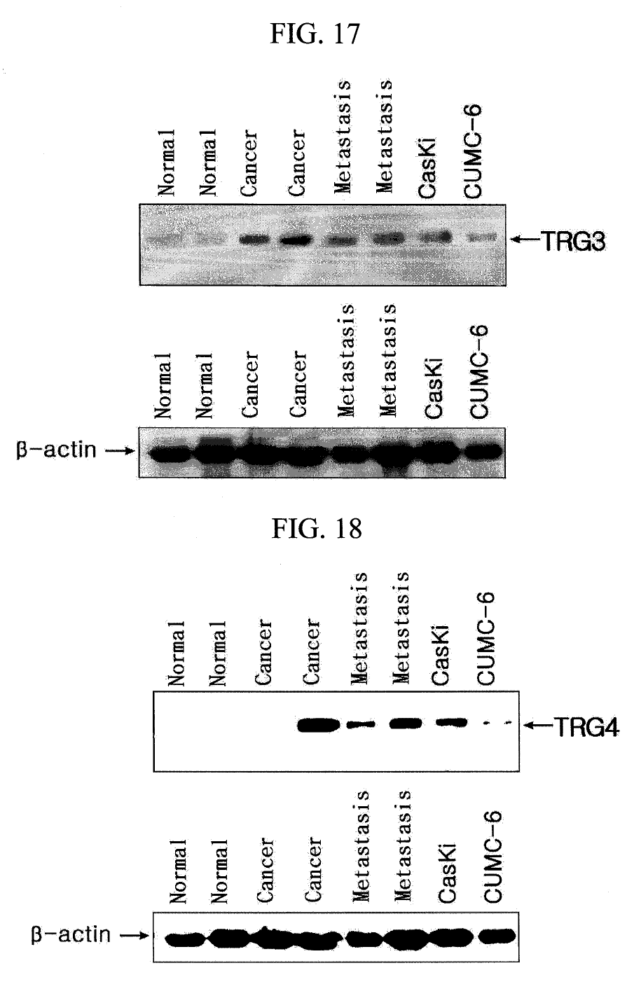

A top of FIG. 17 shows a northern blotting result to determine whether or not the TRG3 protooncogene is expressed in the normal exocervical tissue, the cervical cancer tissue, the metastatic cervical lymph node tissue and the cervical cancer cell lines (CaSki and CUMC-6). As shown in FIG. 17, it was revealed that the expression level of the TRG3 protooncogene was increased, that is, a dominant TRG3 mRNA transcript having a size of approximately 1.7 kb was overexpressed in the cervical cancer tissue and the cervical cancer cell lines CaSki and CUMC-6. In FIG. 17, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 17 shows the northern blotting result indicating whether or not β-actin mRNA is transcribed by hybridizing the same sample with β-actin probe.

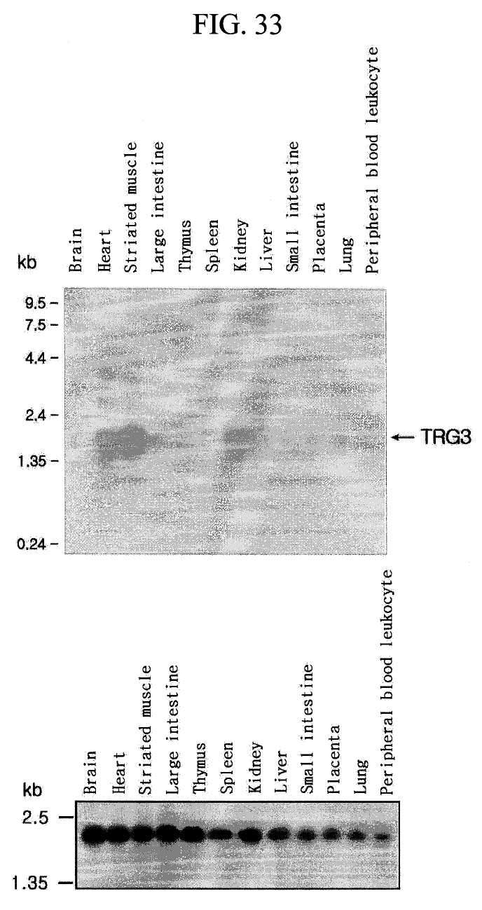

FIG. 33 shows a northern blotting result to determine whether or not the TRG3 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 33 shows the northern blotting result indicating whether or not β-actin mRNA is transcribed by hybridizing the same sample with β-actin probe. As shown in FIG. 33, it was revealed that a TRG3 mRNA transcript (the dominant TRG3 mRNA transcript having a size of approximately 1.7 kb) was expressed in the normal tissues such as the heart and the muscle, but rarely expressed in the normal tissues such as the brain, the large intestine, the thymus, the spleen, the kidney, the liver, the small intestine, the placenta, the lung and the peripheral blood leukocyte.

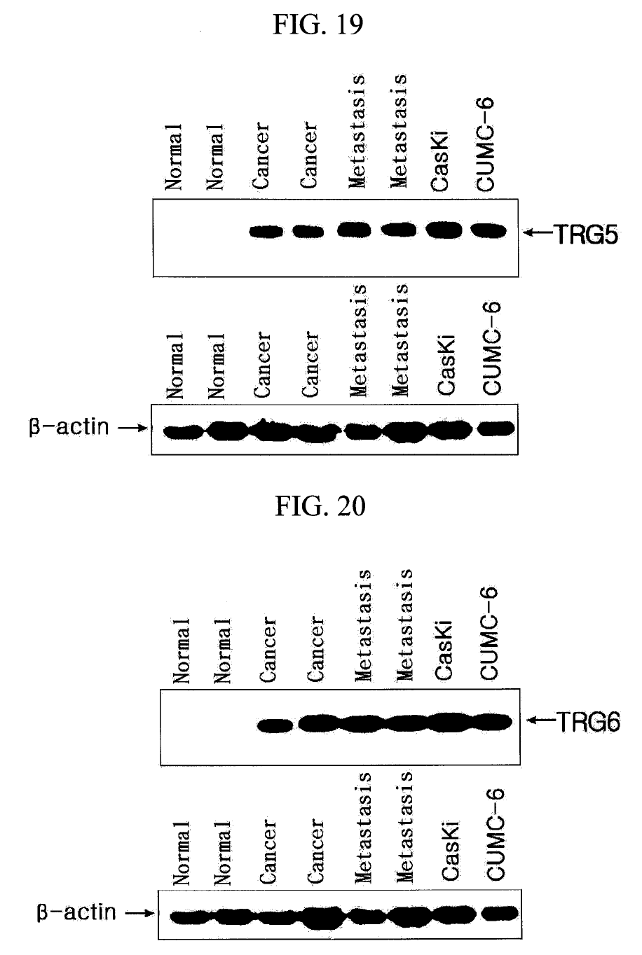

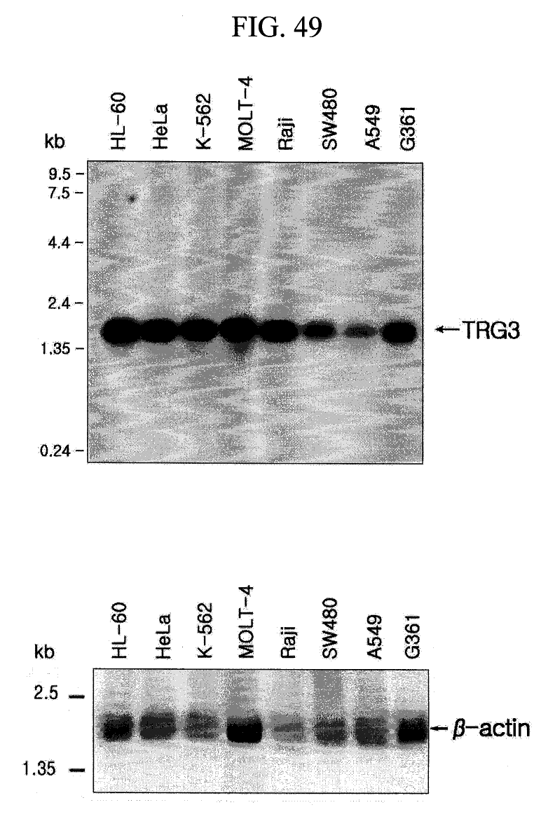

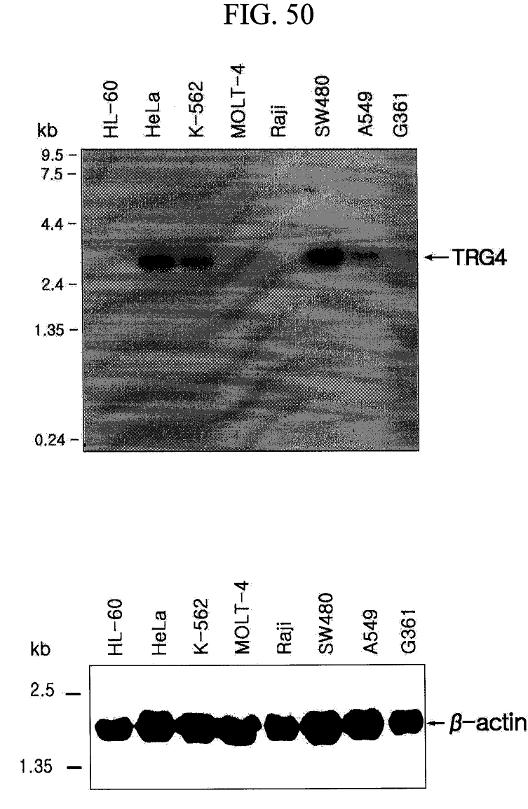

FIG. 49 shows a northern blotting result to determine whether or not the TRG3 protooncogene is expressed in the human cancer cell lines, for example HL-60, HeLa, K-562, MOLT-4, Raji, SW480, A549 and G361 (Clontech). A bottom of FIG. 49 shows the northern blotting result indicating whether or not β-actin mRNA is transcribed by hybridizing the same sample with β-actin probe. As shown in FIG. 19a, it was revealed that a TRG3 mRNA transcript (the dominant TRG3 mRNA transcript having a size of approximately 1.7 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

A top of FIG. 18 shows a northern blotting result to determine whether or not the TRG4 protooncogene is expressed in the normal exocervical tissue, the cervical cancer tissue, the metastatic cervical lymph node tissue and the cervical cancer cell lines (CaSki and CUMC-6). As shown in FIG. 18, it was revealed that the expression level of the TRG4 protooncogene was increased, that is, a dominant TRG4 mRNA transcript having a size of approximately 3.0 kb was overexpressed in the cervical cancer tissue and the cervical cancer cell lines CaSki and CUMC-6. In FIG. 18, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 18 shows the northern blotting result indicating whether or not β-actin mRNA is transcribed by hybridizing the same sample with β-actin probe.

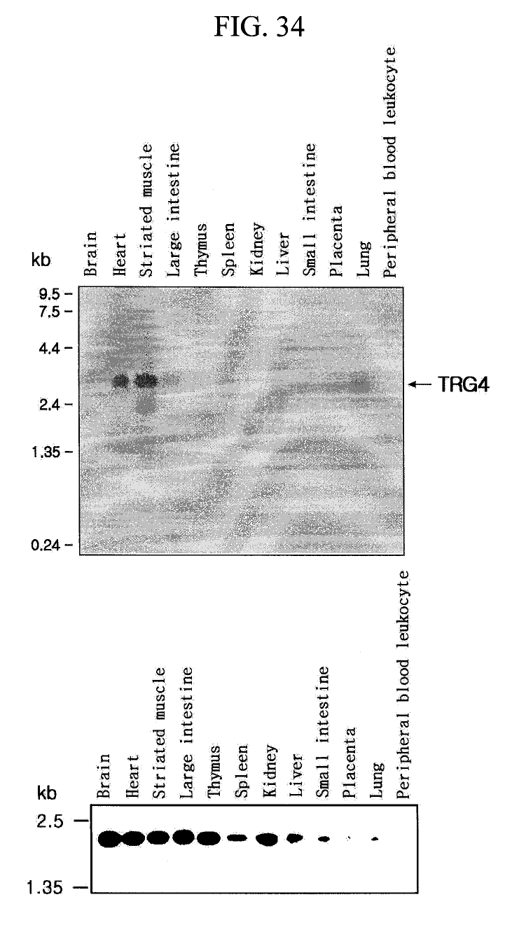

FIG. 34 shows a northern blotting result to determine whether or not the TRG4 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 34 shows the northern blotting result indicating whether or not β-actin mRNA is transcribed by hybridizing the same sample with β-actin probe. As shown in FIG. 34, it was revealed that a TRG4 mRNA transcript (the dominant TRG4 mRNA transcript having a size of approximately 3.0 kb) was expressed in the normal muscle tissue, but very rarely expressed in the normal tissues such as the brain, the heart, the large intestine, the thymus, the spleen, the kidney, the liver, the small intestine, the placenta, the lung and the peripheral blood leukocyte.

FIG. 50 shows a northern blotting result to determine whether or not the TRG4 protooncogene is expressed in the human cancer cell lines, for example HL-60, HeLa, K-562, MOLT-4, Raji, SW480, A549 and G361 (Clontech). A bottom of FIG. 50 shows the northern blotting result indicating whether or not β-actin mRNA is transcribed by hybridizing the same sample with β-actin probe. As shown in FIG. 50, it was revealed that a TRG4 mRNA transcript (the dominant TRG4 mRNA transcript having a size of approximately 3.0 kb) was very highly expressed in the HeLa uterine cancer cell line, the chronic myelogenous leukemia cell line K-562 and the colon cancer cell line SW480.

A top of FIG. 19 shows a northern blotting result to determine whether or not the TRG5 protooncogene is expressed in the normal exocervical tissue, the cervical cancer tissue, the metastatic cervical lymph node tissue and the cervical cancer cell lines (CaSki and CUMC-6). As shown in FIG. 19, it was revealed that the expression level of the TRG5 protooncogene was increased, that is, a dominant TRG5 mRNA transcript having a size of approximately 1.4 kb was overexpressed in the cervical cancer tissue and the cervical cancer cell lines CaSki and CUMC-6. In FIG. 19, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 19 shows the northern blotting result indicating whether or not β-actin mRNA is transcribed by hybridizing the same sample with β-actin probe.

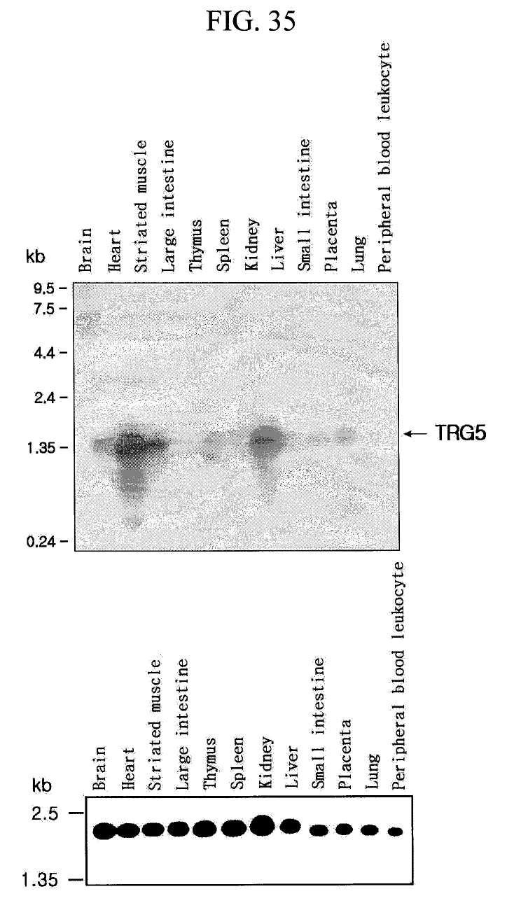

FIG. 35 shows a northern blotting result to determine whether or not the TRG5 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 35 shows the northern blotting result indicating whether or not β-actin mRNA is transcribed by hybridizing the same sample with β-actin probe. As shown in FIG. 35, it was revealed that a TRG5 mRNA transcript (the dominant TRG5 mRNA transcript having a size of approximately 1.4 kb) was expressed in the normal tissues such as the brain, the heart, the muscle and the kidney, but rarely expressed in the normal tissues such as the large intestine, the thymus, the spleen, the liver, the small intestine, the placenta, the lung and the peripheral blood leukocyte.

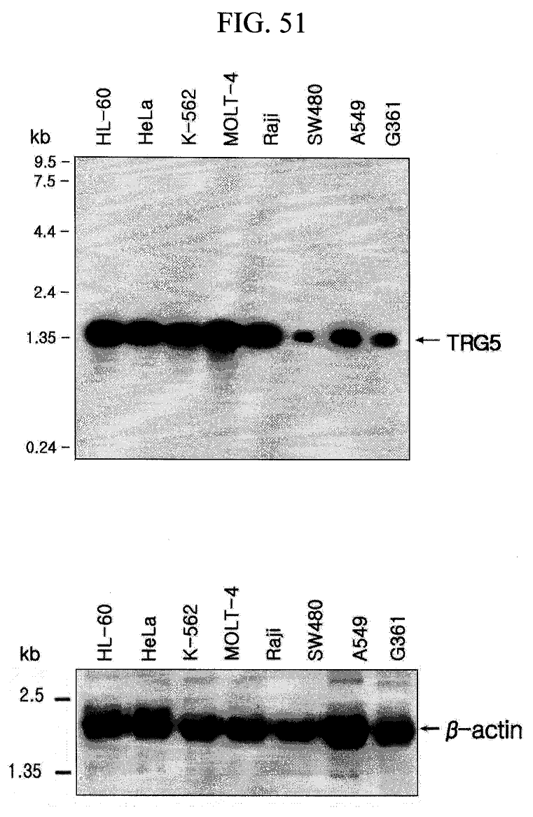

FIG. 51 shows a northern blotting result to determine whether or not the TRG5 protooncogene is expressed in the human cancer cell lines, for example HL-60, HeLa, K-562, MOLT-4, Raji, SW480, A549 and G361 (Clontech). A bottom of FIG. 51 shows the northern blotting result indicating whether or not β-actin mRNA is transcribed by hybridizing the same sample with β-actin probe. As shown in FIG. 51, it was revealed that a TRG5 mRNA transcript (the dominant TRG5 mRNA transcript having a size of approximately 1.4 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

A top of FIG. 20 shows a northern blotting result to determine whether or not the TRG6 protooncogene is expressed in the normal exocervical tissue, the cervical cancer tissue, the metastatic cervical lymph node tissue and the cervical cancer cell lines (CaSki and CUMC-6). As shown in FIG. 20, it was revealed that the expression level of the TRG6 protooncogene was increased, that is, a dominant TRG6 mRNA transcript having a size of approximately 7.0 kb was overexpressed in the cervical cancer tissue and the cervical cancer cell lines CaSki and CUMC-6. In FIG. 20, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 20 shows the northern blotting result indicating whether or not β-actin mRNA is transcribed by hybridizing the same sample with β-actin probe.

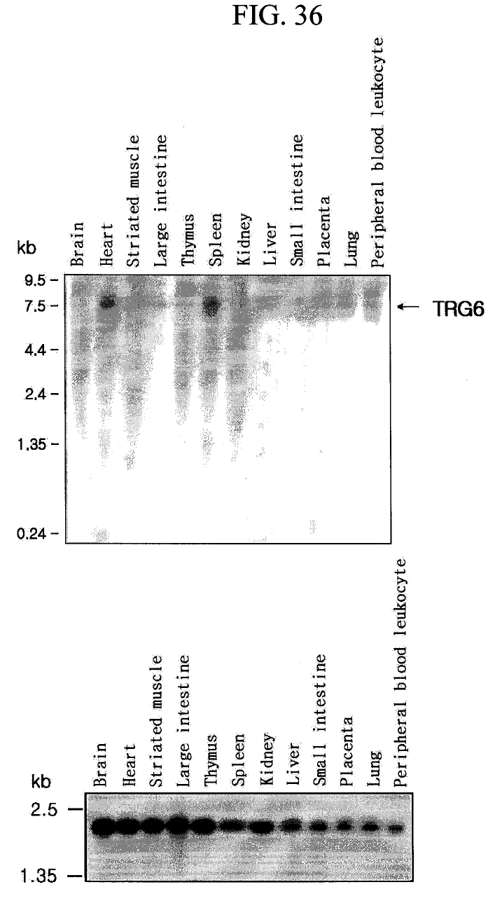

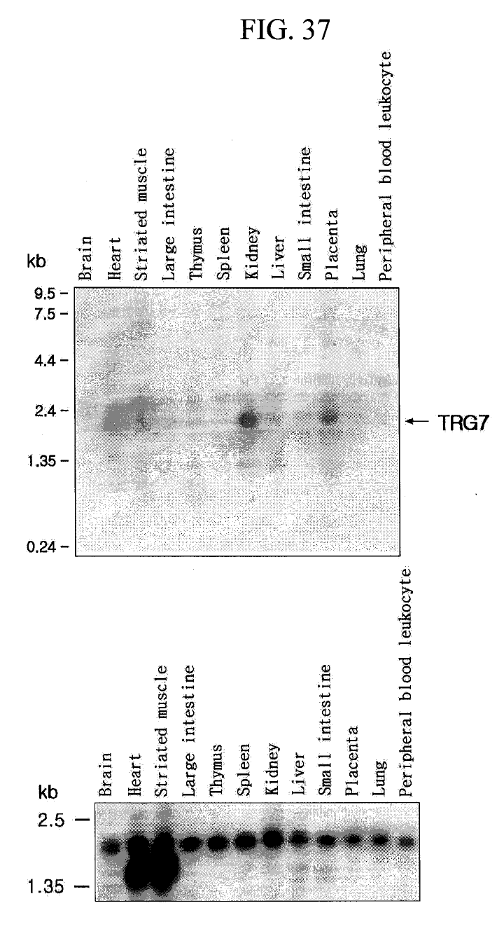

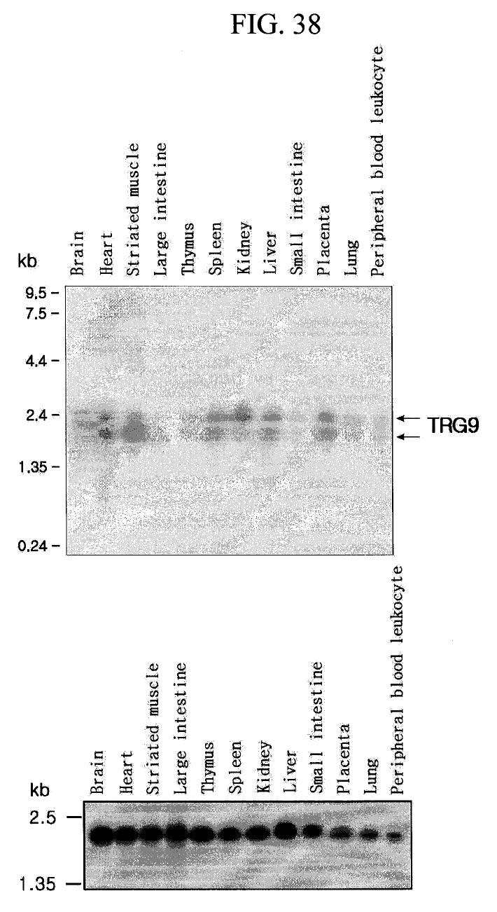

FIG. 36 shows a northern blotting result to determine whether or not the TRG6 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 36 shows the northern blotting result indicating whether or not β-actin mRNA is transcribed by hybridizing the same sample with β-actin probe. As shown in FIG. 36, it was revealed that a TRG6 mRNA transcript (the dominant TRG6 mRNA transcript having a size of approximately 7.0 kb) was not expressed in the normal tissues such as the brain, the heart, the muscle, the large intestine, the thymus, the spleen, the kidney, the liver, the small intestine, the placenta, the lung and the peripheral blood leukocyte.

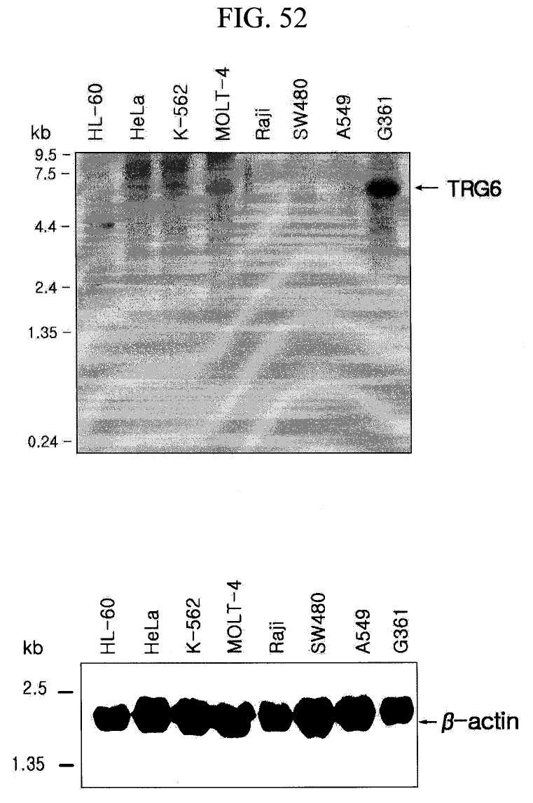

FIG. 52 shows a northern blotting result to determine whether or not the TRG6 protooncogene is expressed in the human cancer cell lines, for example the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361 (Clontech). A bottom of FIG. 52 shows the northern blotting result indicating whether or not β-actin mRNA is transcribed by hybridizing the same sample with β-actin probe. As shown in FIG. 52, it was revealed that a TRG6 mRNA transcript (the dominant TRG6 mRNA transcript having a size of approximately 7.0 kb) was very highly expressed in the HeLa uterine cancer cell line and the skin cancer cell line G361.

A top of FIG. 21 shows a northern blotting result to determine whether or not the TRG7 protooncogene is expressed in the normal exocervical tissue, the cervical cancer tissue, the metastatic cervical lymph node tissue and the cervical cancer cell lines (CaSki and CUMC-6). As shown in FIG. 21, it was revealed that the expression level of the TRG7 protooncogene was increased, that is, a dominant TRG7 mRNA transcript having a size of approximately 2.0 kb was overexpressed in the cervical cancer tissue and the cervical cancer cell lines CaSki and CUMC-6. In FIG. 21, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 17 shows the northern blotting result indicating whether or not β-actin mRNA is transcribed by hybridizing the same sample with β-actin probe.

FIG. 37 shows a northern blotting result to determine whether or not the TRG7 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 37 shows the northern blotting result indicating whether or not β-actin mRNA is transcribed by hybridizing the same sample with β-actin probe. As shown in FIG. 37, it was revealed that a TRG7 mRNA transcript (the dominant TRG7 mRNA transcript having a size of approximately 2.0 kb) was expressed in the normal tissues such as the heart, the muscle, the kidney and the placenta, and a dominant TRG7 mRNA transcript having a size of approximately 2.4 kb was also rarely expressed at the same time, but not expressed in the normal tissues such as the brain, the large intestine, the thymus, the spleen, the liver, the small intestine, the lung and the peripheral blood leukocyte.

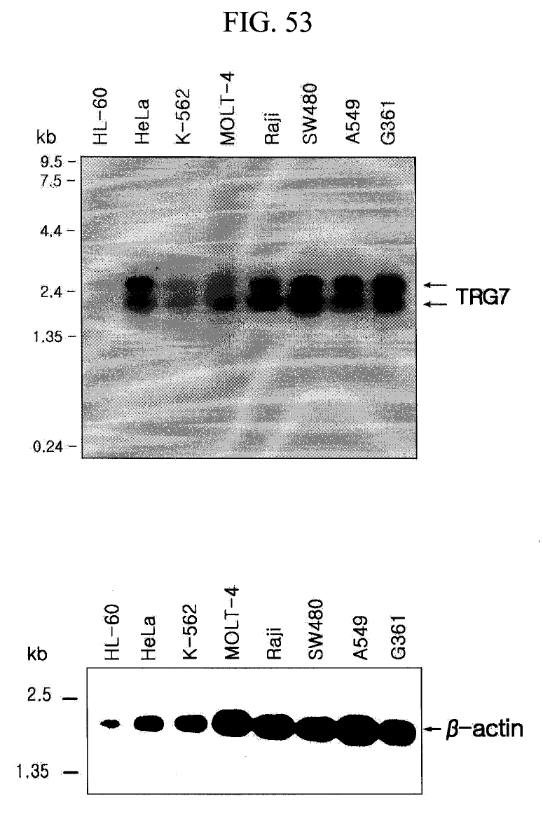

FIG. 53 shows a northern blotting result to determine whether or not the TRG7 protooncogene is expressed in the human cancer cell lines, for example HL-60, HeLa, K-562, MOLT-4, Raji, SW480, A549 and G361 (Clontech). A bottom of FIG. 53 shows the northern blotting result indicating whether or not β-actin mRNA is transcribed by hybridizing the same sample with β-actin probe. As shown in FIG. 53, it was revealed that a TRG7 mRNA transcript (the dominant TRG7 mRNA transcript having a size of approximately 2.0 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. At the same time, a TRG7 mRNA transcript having a size of approximately 2.4 kb was also overexpressed in the human cancer cell lines.

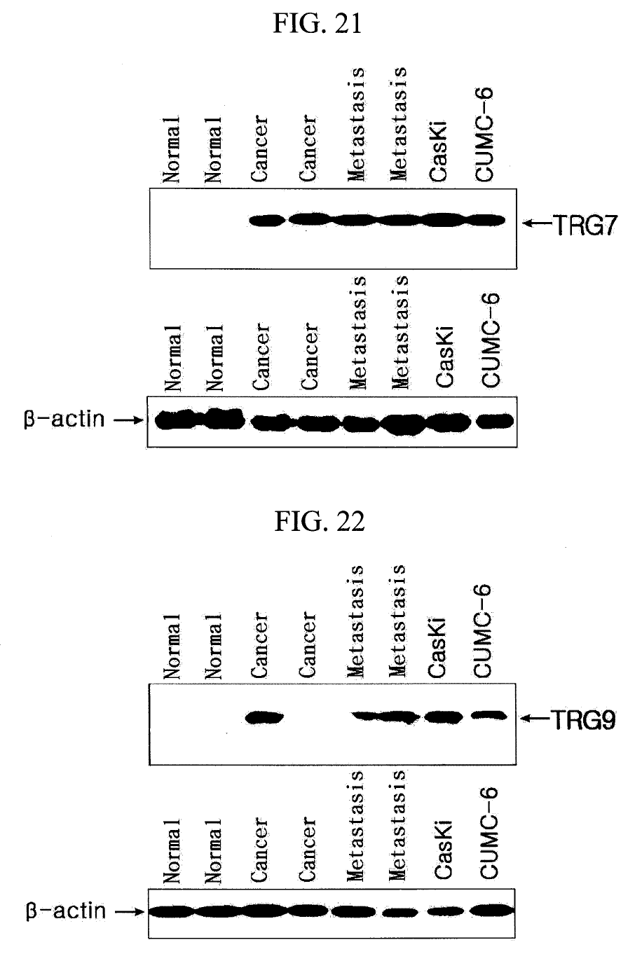

A top of FIG. 22 shows a northern blotting result to determine whether or not the TRG9 protooncogene is expressed in the normal exocervical tissue, the cervical cancer tissue, the metastatic cervical lymph node tissue and the cervical cancer cell lines (CaSki and CUMC-6). As shown in FIG. 22, it was revealed that the expression level of the TRG9 protooncogene was increased, that is, a dominant TRG9 mRNA transcript having a size of approximately 2.4 kb was overexpressed in the cervical cancer tissue and the cervical cancer cell lines CaSki and CUMC-6. In FIG. 22, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 22 shows the northern blotting result indicating whether or not β-actin mRNA is transcribed by hybridizing the same sample with β-actin probe.

FIG. 38 shows a northern blotting result to determine whether or not the TRG9 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 38 shows the northern blotting result indicating whether or not β-actin mRNA is transcribed by hybridizing the same sample with β-actin probe. As shown in FIG. 38, it was revealed that a TRG9 mRNA transcript (the dominant TRG3 mRNA transcript having a size of approximately 2.4 kb) was rarely expressed in the normal tissues such as the heart, the muscle, the kidney and the placenta, but hardly expressed in the normal tissues such as the brain, the large intestine, the thymus, the spleen, the liver, the small intestine, the lung and the peripheral blood leukocyte.

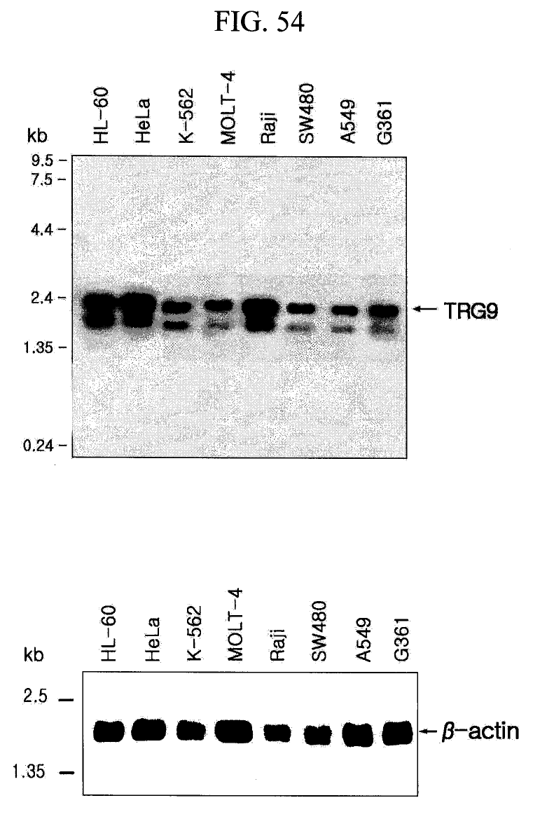

FIG. 54 shows a northern blotting result to determine whether or not the TRG9 protooncogene is expressed in the human cancer cell lines, for example HL-60, HeLa, K-562, MOLT-4, Raji, SW480, A549 and G361 (Clontech). A bottom of FIG. 54 shows the northern blotting result indicating whether or not β-actin mRNA is transcribed by hybridizing the same sample with β-actin probe. As shown in FIG. 54, it was revealed that a TRG3 mRNA transcript (the dominant TRG9 mRNA transcript having a size of approximately 2.4 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, a TRG9 mRNA transcript having a size of approximately 2.0 kb was expressed at the same time.

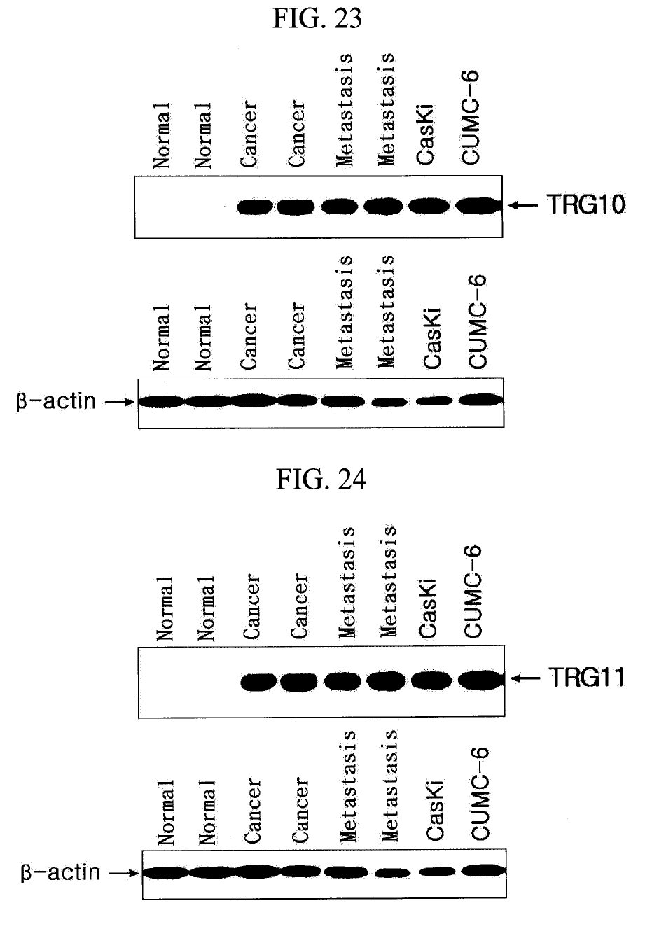

A top of FIG. 23 shows a northern blotting result to determine whether or not the TRG10 protooncogene is expressed in the normal exocervical tissue, the cervical cancer tissue, the metastatic cervical lymph node tissue and the cervical cancer cell lines (CaSki and CUMC-6). As shown in FIG. 23, it was revealed that the expression level of the TRG3 protooncogene was increased, that is, a dominant TRG10 mRNA transcript having a size of approximately 6.0 kb was overexpressed in the cervical cancer tissue and the cervical cancer cell lines CaSki and CUMC-6. In FIG. 23, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 23 shows the northern blotting result indicating whether or not β-actin mRNA is transcribed by hybridizing the same sample with β-actin probe.

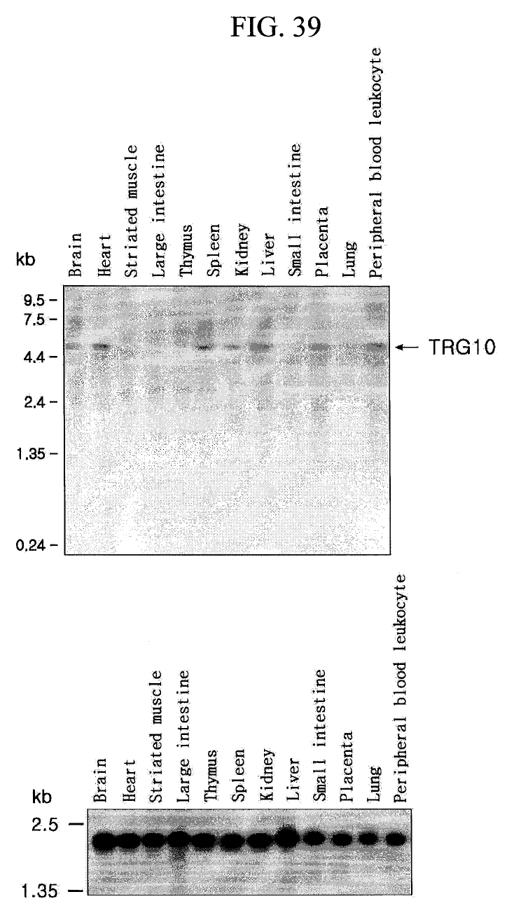

FIG. 39 shows a northern blotting result to determine whether or not the TRG10 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 39 shows the northern blotting result indicating whether or not β-actin mRNA is transcribed by hybridizing the same sample with β-actin probe. As shown in FIG. 39, it was revealed that a TRG10 mRNA transcript (the dominant TRG10 mRNA transcript having a size of approximately 6.0 kb) was expressed in the normal tissues such as the heart, the liver, the peripheral blood leukocyte and the spleen, but not expressed in the normal tissues such as the brain, the muscle, the large intestine, the thymus, the kidney, the small intestine, the placenta and the lung.

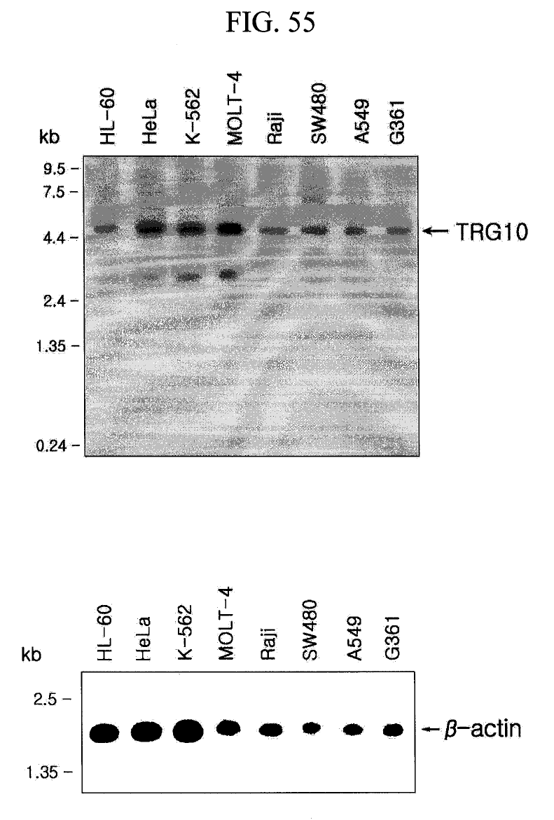

FIG. 55 shows a northern blotting result to determine whether or not the TRG10 protooncogene is expressed in the human cancer cell lines, for example HL-60, HeLa, K-562, MOLT-4, Raji, SW480, A549 and G361 (Clontech). A bottom of FIG. 55 shows the northern blotting result indicating whether or not β-actin mRNA is transcribed by hybridizing the same sample with β-actin probe. As shown in FIG. 55, it was revealed that a TRG10 mRNA transcript (the dominant TRG10 mRNA transcript having a size of approximately 6.0 kb) was very highly expressed in the HeLa uterine cancer cell line, the chronic myelogenous leukemia cell line K-562 and the lymphoblastic leukaemia cell line MOLT-4, and highly expressed in the promyelocyte leukemia cell line HL-60, the Burkitt lymphoma cell line Raji, the colon cancer cell line SW480, the lung cancer cell line A549 and the skin cancer cell line G361. Also, a TRG10 mRNA transcript having a size of approximately 3.0 kb was expressed in the cancer cell lines.

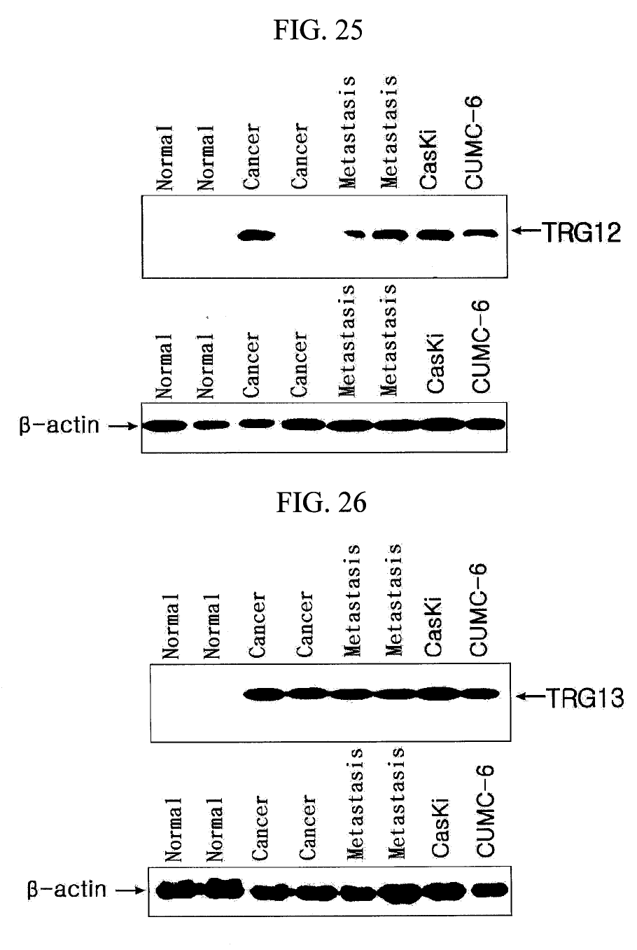

A top of FIG. 24 shows a northern blotting result to determine whether or not the TRG11 protooncogene is expressed in the normal exocervical tissue, the cervical cancer tissue, the metastatic cervical lymph node tissue and the cervical cancer cell lines (CaSki and CUMC-6). As shown in FIG. 24, it was revealed that the expression level of the TRG11 protooncogene was increased, that is, a dominant TRG11 mRNA transcript having a size of approximately 1.5 kb was overexpressed in the cervical cancer tissue, the metastatic cervical cancer tissue and the cervical cancer cell lines CaSki and CUMC-6. In FIG. 24, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 24 shows the northern blotting result indicating whether or not β-actin mRNA is transcribed by hybridizing the same sample with β-actin probe.

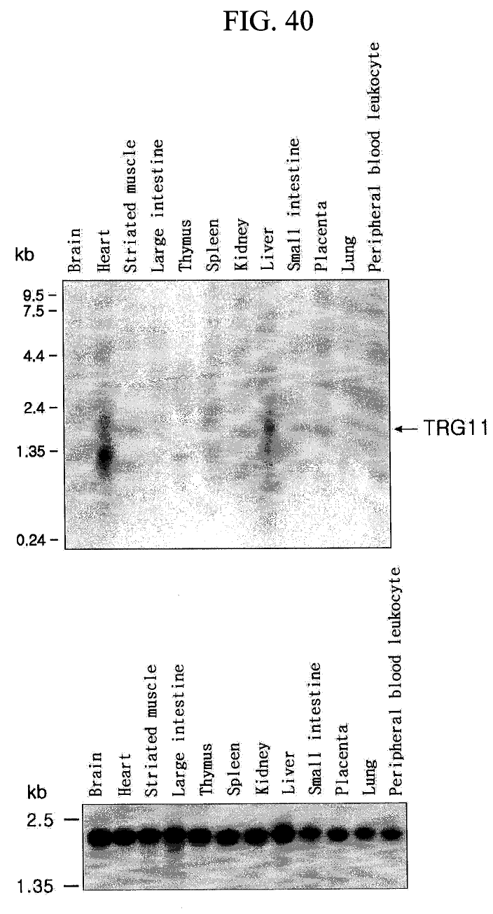

FIG. 40 shows a northern blotting result to determine whether or not the TRG11 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 40 shows the northern blotting result indicating whether or not β-actin mRNA is transcribed by hybridizing the same sample with β-actin probe. As shown in FIG. 40, it was revealed that a TRG11 mRNA transcript (the dominant TRG11 mRNA transcript having a size of approximately 1.5 kb) was expressed in the normal tissues such as the heart and the liver, but hardly expressed in the normal tissues such as the brain, the muscle, the large intestine, the thymus, the spleen, the kidney, the small intestine, the placenta, the lung and the peripheral blood leukocyte.

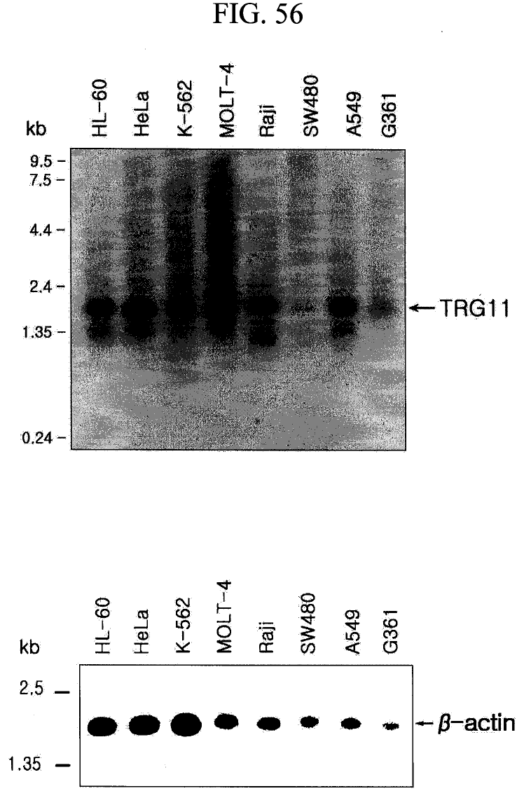

FIG. 56 shows a northern blotting result to determine whether or not the TRG11 protooncogene is expressed in the human cancer cell lines, for example HL-60, HeLa, K-562, MOLT-4, Raji, SW480, A549 and G361 (Clontech). A bottom of FIG. 56 shows the northern blotting result indicating whether or not β-actin mRNA is transcribed by hybridizing the same sample with β-actin probe. As shown in FIG. 56, it was revealed that a TRG11 mRNA transcript (the dominant TRG11 mRNA transcript having a size of approximately 1.5 kb) was highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, a TRG11 mRNA transcript having a size of approximately 1.3 kb was rarely expressed in the cancer cell lines.

A top of FIG. 25 shows a northern blotting result to determine whether or not the TRG12 protooncogene is expressed in the normal exocervical tissue, the cervical cancer tissue, the metastatic cervical lymph node tissue and the cervical cancer cell lines (CaSki and CUMC-6). As shown in FIG. 25, it was revealed that the expression level of the TRG12 protooncogene was increased, that is, a dominant TRG12 mRNA transcript having a size of approximately 5.0 kb was overexpressed in the cervical cancer tissue and the cervical cancer cell lines CaSki and CUMC-6. In FIG. 25, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 25 shows the northern blotting result indicating whether or not β-actin mRNA is transcribed by hybridizing the same sample with β-actin probe.

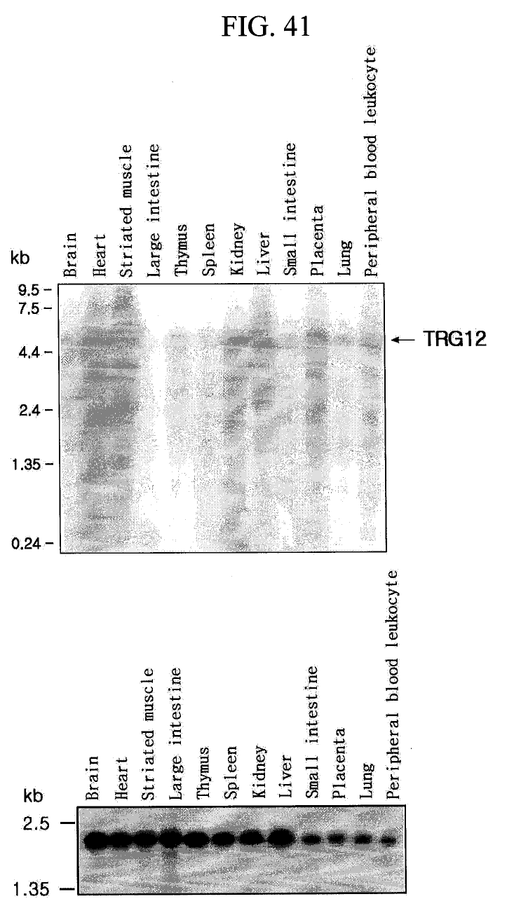

FIG. 41 shows a northern blotting result to determine whether or not the TRG12 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 41 shows the northern blotting result indicating whether or not β-actin mRNA is transcribed by hybridizing the same sample with β-actin probe. As shown in FIG. 41, it was revealed that a TRG12 mRNA transcript (the dominant TRG12 mRNA transcript having a size of approximately 5.0 kb) was rarely expressed in the normal tissues such as the brain, the heart, the muscle, the kidney, the liver, the placenta and the peripheral blood leukocyte, but hardly expressed in the normal tissues such as the large intestine, the thymus, the spleen, the small intestine and the lung.

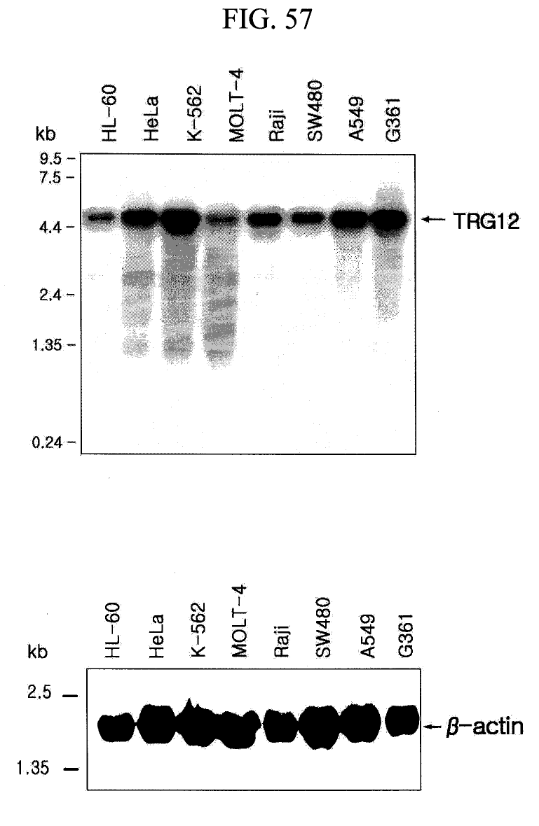

FIG. 57 shows a northern blotting result to determine whether or not the TRG12 protooncogene is expressed in the human cancer cell lines, for example HL-60, HeLa, K-562, MOLT-4, Raji, SW480, A549 and G361 (Clontech). A bottom of FIG. 57 shows the northern blotting result indicating whether or not β-actin mRNA is transcribed by hybridizing the same sample with β-actin probe. As shown in FIG. 57, it was revealed that a TRG12 mRNA transcript (the dominant TRG12 mRNA transcript having a size of approximately 5.0 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

A top of FIG. 26 shows a northern blotting result to determine whether or not the TRG13 protooncogene is expressed in the normal exocervical tissue, the cervical cancer tissue, the metastatic cervical lymph node tissue and the cervical cancer cell lines (CaSki and CUMC-6). As shown in FIG. 26, it was revealed that the expression level of the TRG13 protooncogene was increased, that is, a dominant TRG13 mRNA transcript having a size of approximately 4.0 kb was overexpressed in the cervical cancer tissue and the cervical cancer cell lines CaSki and CUMC-6. In FIG. 26, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 26 shows the northern blotting result indicating whether or not β-actin mRNA is transcribed by hybridizing the same sample with β-actin probe.

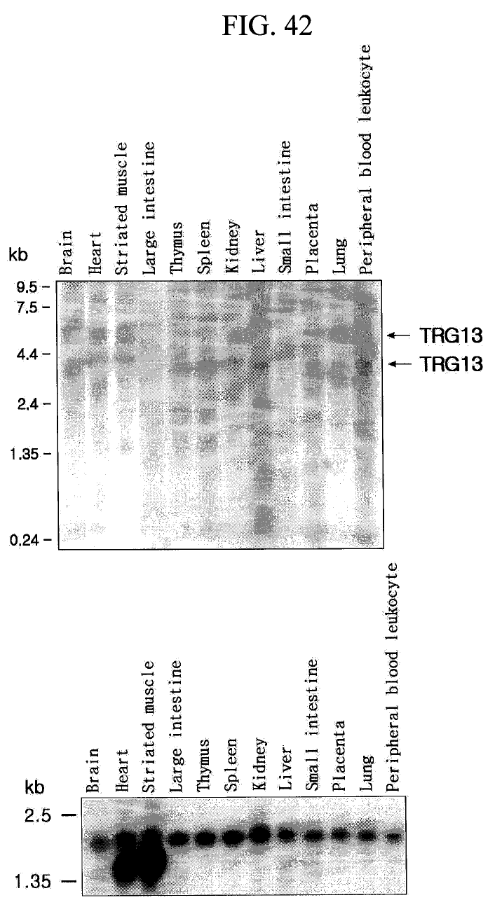

FIG. 42 shows a northern blotting result to determine whether or not the TRG13 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 42 shows the northern blotting result indicating whether or not β-actin mRNA is transcribed by hybridizing the same sample with β-actin probe. As shown in FIG. 42, it was revealed that a TRG13 mRNA transcript (the dominant TRG13 mRNA transcript having a size of approximately 4.0 kb) was expressed in the normal tissues such as the heart and the muscle, but rarely expressed in the normal tissues such as the brain, the large intestine, the thymus, the spleen, the kidney, the liver, the small intestine, the placenta, the lung and the peripheral blood leukocyte. Also, a TRG13 mRNA transcript having a size of approximately 5.0 kb was rarely expressed at the same time.

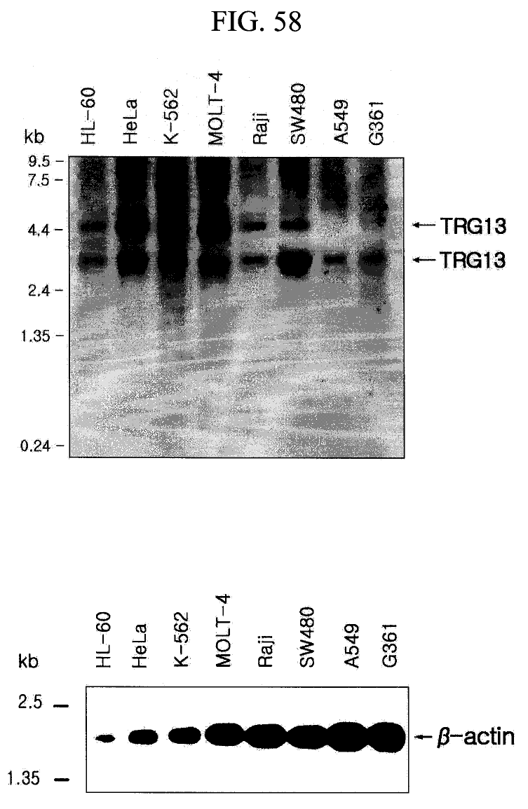

FIG. 58 shows a northern blotting result to determine whether or not the TRG13 protooncogene is expressed in the human cancer cell lines, for example HL-60, HeLa, K-562, MOLT-4, Raji, SW480, A549 and G361 (Clontech). A bottom of FIG. 58 shows the northern blotting result indicating whether or not β-actin mRNA is transcribed by hybridizing the same sample with β-actin probe. As shown in FIG. 58, it was revealed that a TRG13 mRNA transcript (the dominant TRG13 mRNA transcript having a size of approximately 4.0 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, a TRG13 mRNA transcript having a size of approximately 5.0 kb was highly expressed at the same time.

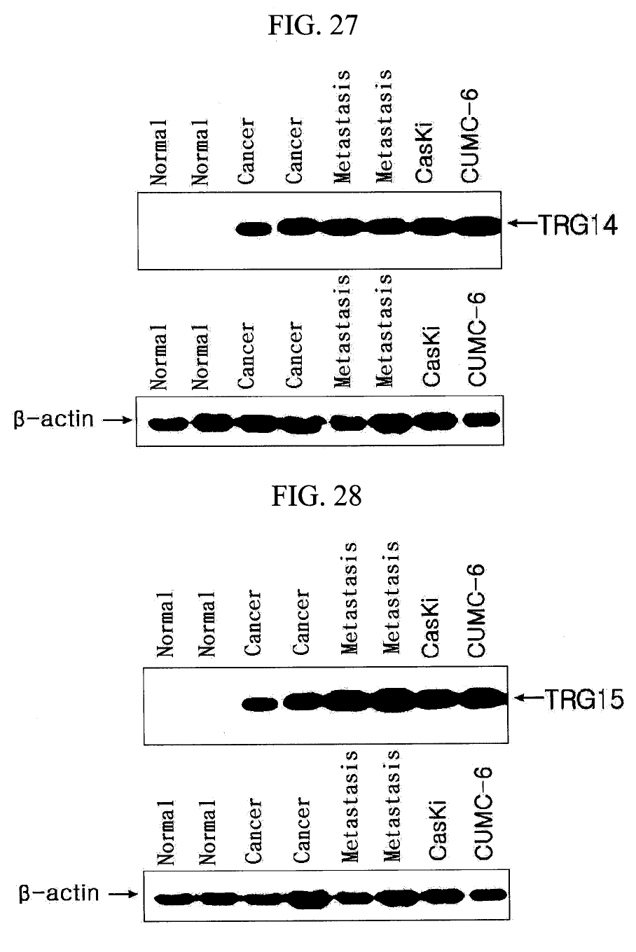

A top of FIG. 27 shows a northern blotting result to determine whether or not the TRG14 protooncogene is expressed in the normal exocervical tissue, the cervical cancer tissue, the metastatic cervical lymph node tissue and the cervical cancer cell lines (CaSki and CUMC-6). As shown in FIG. 27, it was revealed that the expression level of the TRG14 protooncogene was increased, that is, a dominant TRG14 mRNA transcript having a size of approximately 3.5 kb was overexpressed in the cervical cancer tissue and the cervical cancer cell lines CaSki and CUMC-6. In FIG. 27, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 27 shows the northern blotting result indicating whether or not β-actin mRNA is transcribed by hybridizing the same sample with β-actin probe.

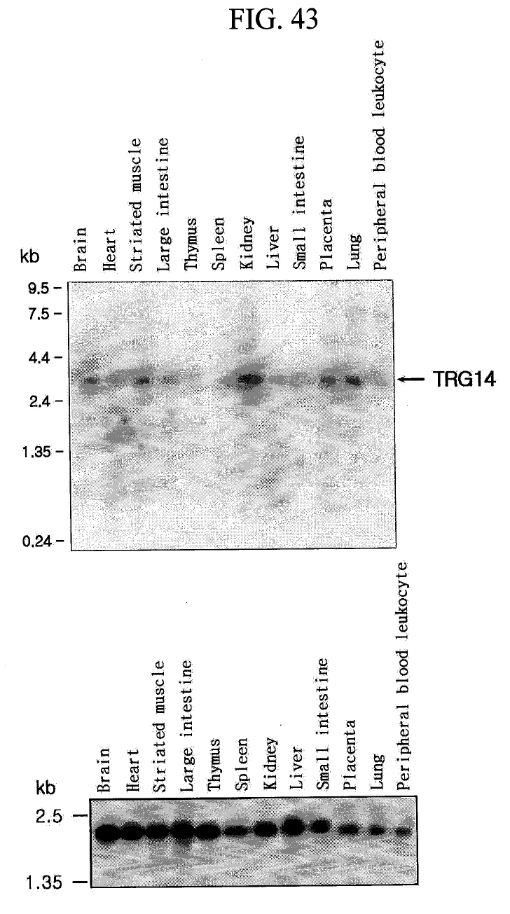

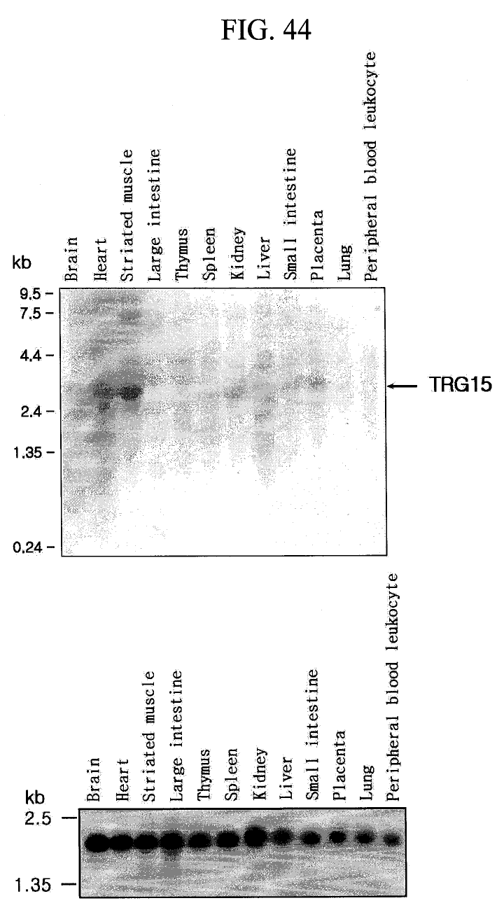

FIG. 43 shows a northern blotting result to determine whether or not the TRG14 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 43 shows the northern blotting result indicating whether or not β-actin mRNA is transcribed by hybridizing the same sample with β-actin probe. As shown in FIG. 43, it was revealed that a TRG14 mRNA transcript (the dominant TRG14 mRNA transcript having a size of approximately 3.5 kb) was expressed in the normal tissues such as the heart and the muscle, but rarely expressed in the normal tissues such as the brain, the large intestine, the thymus, the spleen, the kidney, the liver, the small intestine, the placenta, the lung and the peripheral blood leukocyte.

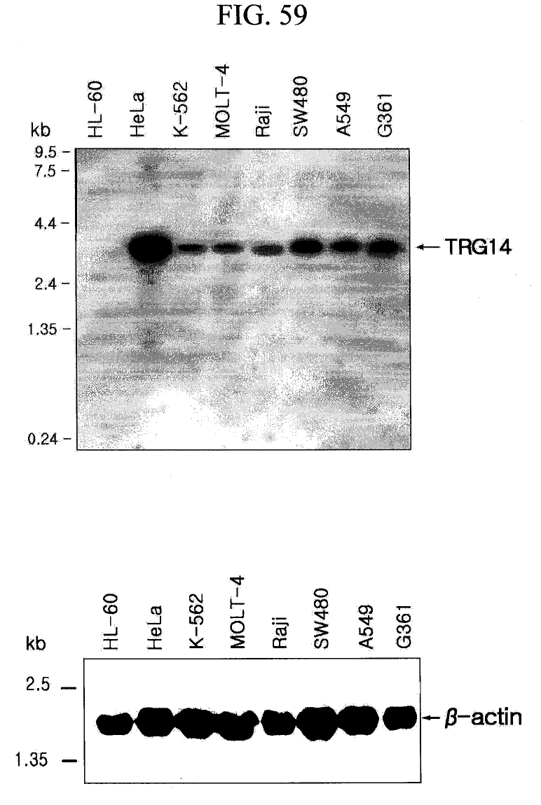

FIG. 59 shows a northern blotting result to determine whether or not the TRG14 protooncogene is expressed in the human cancer cell lines, for example HL-60, HeLa, K-562, MOLT-4, Raji, SW480, A549 and G361 (Clontech). A bottom of FIG. 59 shows the northern blotting result indicating whether or not β-actin mRNA is transcribed by hybridizing the same sample with β-actin probe. As shown in FIG. 59, it was revealed that a TRG14 mRNA transcript (the dominant TRG14 mRNA transcript having a size of approximately 3.5 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

A top of FIG. 28 shows a northern blotting result to determine whether or not the TRG15 protooncogene is expressed in the normal exocervical tissue, the cervical cancer tissue, the metastatic cervical lymph node tissue and the cervical cancer cell lines (CaSki and CUMC-6). As shown in FIG. 28, it was revealed that the expression level of the TRG15 protooncogene was increased, that is, a dominant TRG15 mRNA transcript having a size of approximately 3.5 kb was overexpressed in the cervical cancer tissue and the cervical cancer cell lines CaSki and CUMC-6. In FIG. 28, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 28 shows the northern blotting result indicating whether or not β-actin mRNA is transcribed by hybridizing the same sample with β-actin probe

FIG. 44 shows a northern blotting result to determine whether or not the TRG15 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 44 shows the northern blotting result indicating whether or not β-actin mRNA is transcribed by hybridizing the same sample with β-actin probe. As shown in FIG. 44, it was revealed that a TRG15 mRNA transcript (the dominant TRG15 mRNA transcript having a size of approximately 3.5 kb) was expressed in the normal tissues such as the heart and the muscle, but very rarely expressed in the normal tissues such as the brain, the large intestine, the thymus, the spleen, the kidney, the liver, the small intestine, the placenta, the lung and the peripheral blood leukocyte. Also, TRG15 mRNA transcripts having sizes of approximately 3.0 kb and 4.0 kb were very rarely expressed at the same time.

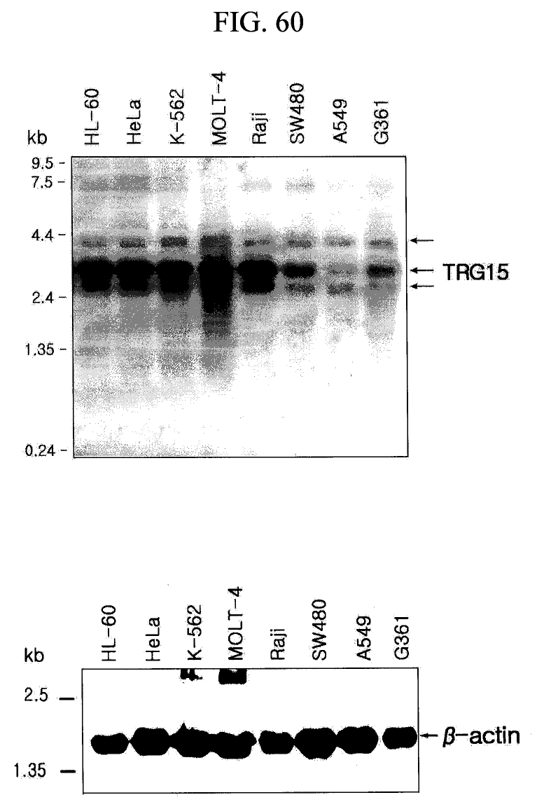

FIG. 60 shows a northern blotting result to determine whether or not the TRG15 protooncogene is expressed in the human cancer cell lines, for example HL-60, HeLa, K-562, MOLT-4, Raji, SW480, A549 and G361 (Clontech). A bottom of FIG. 60 shows the northern blotting result indicating whether or not β-actin mRNA is transcribed by hybridizing the same sample with β-actin probe. As shown in FIG. 60, it was revealed that a TRG15 mRNA transcript (the dominant TRG15 mRNA transcript having a size of approximately 3.5 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, TRG15 mRNA transcripts having sizes of approximately 3.0 kb and 4.0 kb were highly expressed at the same time.

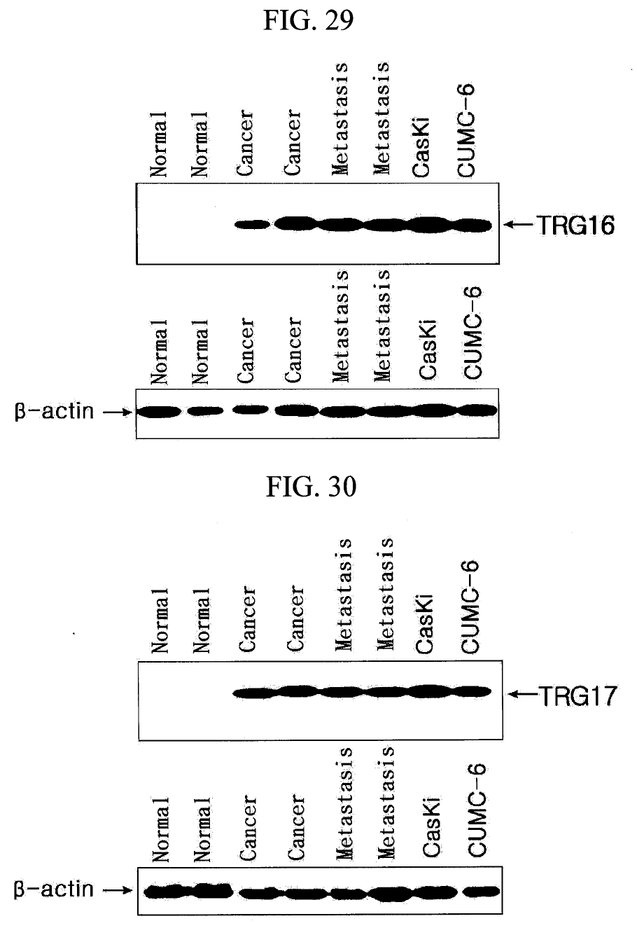

A top of FIG. 29 shows a northern blotting result to determine whether or not the TRG16 protooncogene is expressed in the normal exocervical tissue, the cervical cancer tissue, the metastatic cervical lymph node tissue and the cervical cancer cell lines (CaSki and CUMC-6). As shown in FIG. 29, it was revealed that the expression level of the TRG16 protooncogene was increased, that is, a dominant TRG16 mRNA transcript having a size of approximately 4.5 kb was overexpressed in the cervical cancer tissue and the cervical cancer cell lines CaSki and CUMC-6. In FIG. 29, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 29 shows the northern blotting result indicating whether or not β-actin mRNA is transcribed by hybridizing the same sample with β-actin probe.

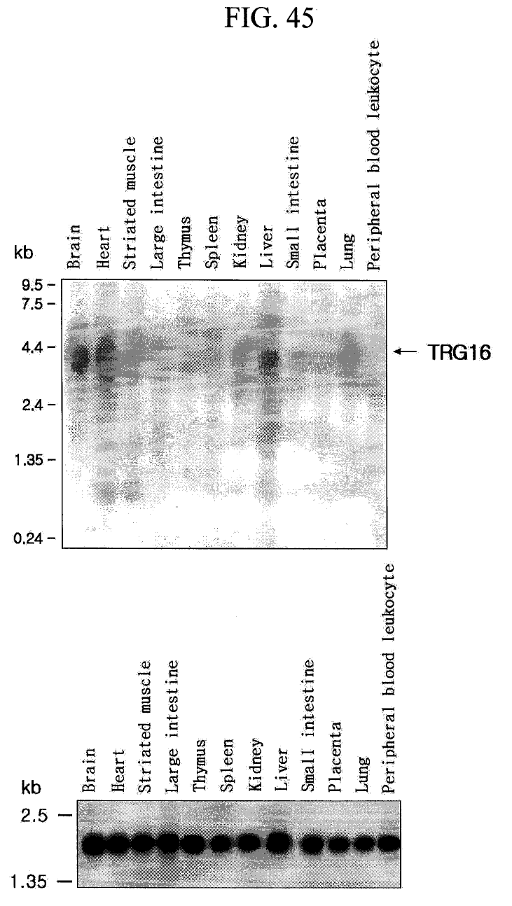

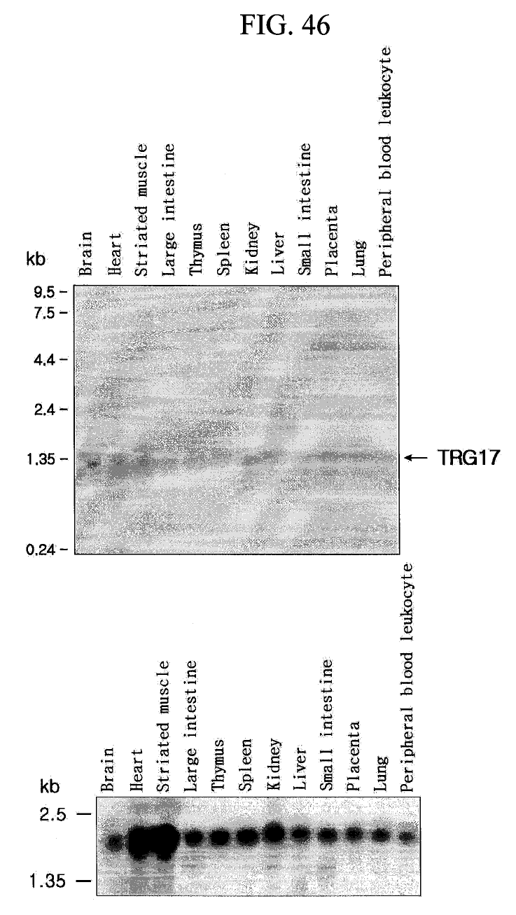

FIG. 45 shows a northern blotting result to determine whether or not the TRG16 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 45 shows the northern blotting result indicating whether or not β-actin mRNA is transcribed by hybridizing the same sample with β-actin probe. As shown in FIG. 45, it was revealed that a TRG16 mRNA transcript (the dominant TRG16 mRNA transcript having a size of approximately 3.5 kb) was expressed in the normal tissues such as the brain and the heart, but very rarely expressed in the normal tissues such as the muscle, the large intestine, the thymus, the spleen, the kidney, the liver, the small intestine, the placenta, the lung and the peripheral blood leukocyte. Also, a TRG16 mRNA transcript having a size of approximately 5.0 kb was very rarely expressed at the same time.

FIG. 61 shows a northern blotting result to determine whether or not the TRG16 protooncogene is expressed in the human cancer cell lines, for example HL-60, HeLa, K-562, MOLT-4, Raji, SW480, A549 and G361 (Clontech). A bottom of FIG. 61 shows the northern blotting result indicating whether or not β-actin mRNA is transcribed by hybridizing the same sample with β-actin probe. As shown in FIG. 61, it was revealed that a TRG16 mRNA transcript (the dominant TRG16 mRNA transcript having a size of approximately 4.5 kb) was very highly expressed in the HeLa uterine cancer cell line, the chronic myelogenous leukemia cell line K-562, the lymphoblastic leukaemia cell line MOLT-4, the colon cancer cell line SW480, the lung cancer cell line A549 and the skin cancer cell line G361. Also, a TRG16 mRNA transcript having a size of approximately 5.0 kb was highly expressed at the same time.

A top of FIG. 30 shows a northern blotting result to determine whether or not the TRG17 protooncogene is expressed in the normal exocervical tissue, the cervical cancer tissue, the metastatic cervical lymph node tissue and the cervical cancer cell lines (CaSki and CUMC-6). As shown in FIG. 30, it was revealed that the expression level of the TRG17 protooncogene was increased, that is, a dominant TRG17 mRNA transcript having a size of approximately 1.3 kb was overexpressed in the cervical cancer tissue and the cervical cancer cell lines CaSki and CUMC-6. In FIG. 30, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 30 shows the northern blotting result indicating whether or not β-actin mRNA is transcribed by hybridizing the same sample with β-actin probe.

FIG. 46 shows a northern blotting result to determine whether or not the TRG17 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 46 shows the northern blotting result indicating whether or not β-actin mRNA is transcribed by hybridizing the same sample with β-actin probe. As shown in FIG. 46, it was revealed that a TRG17 mRNA transcript (the dominant TRG17 mRNA transcript having a size of approximately 1.3 kb) was expressed in the normal tissues such as the brain, the heart and the muscle, but very rarely expressed in the normal tissues such as the large intestine, the thymus, the spleen, the kidney, the liver, the small intestine, the placenta, the lung and the peripheral blood leukocyte.

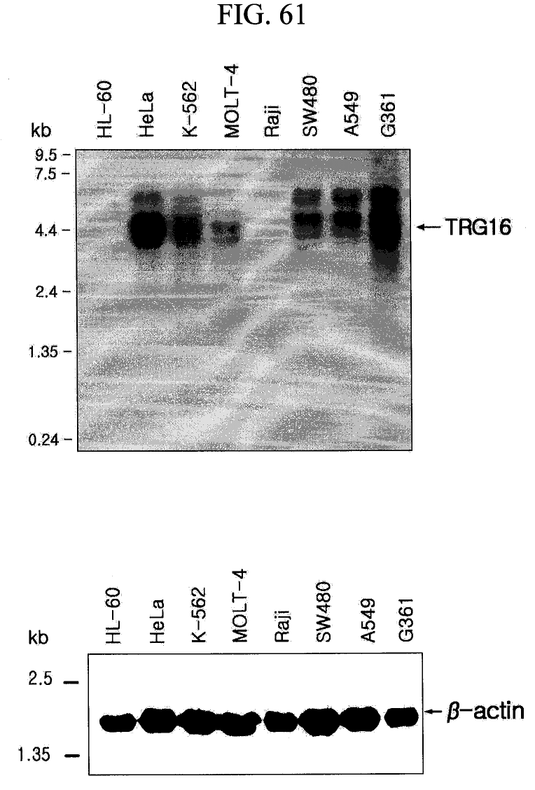

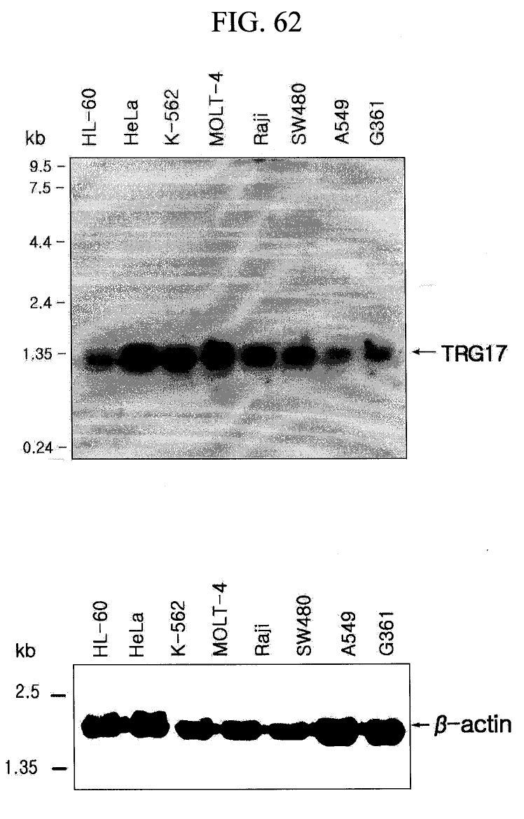

FIG. 62 shows a northern blotting result to determine whether or not the TRG17 protooncogene is expressed in the human cancer cell lines, for example HL-60, HeLa, K-562, MOLT-4, Raji, SW480, A549 and G361 (Clontech). A bottom of FIG. 62 shows the northern blotting result indicating whether or not β-actin mRNA is transcribed by hybridizing the same sample with β-actin probe. As shown in FIG. 62, it was revealed that a TRG17 mRNA transcript (the dominant TRG17 mRNA transcript having a size of approximately 1.3 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

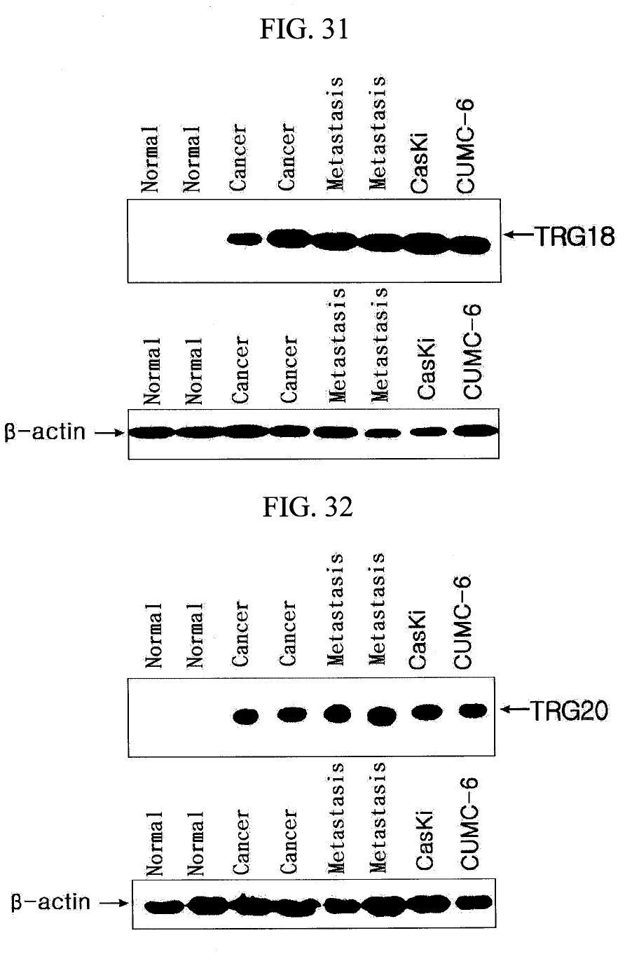

A top of FIG. 31 shows a northern blotting result to determine whether or not the TRG18 protooncogene is expressed in the normal exocervical tissue, the cervical cancer tissue, the metastatic cervical lymph node tissue and the cervical cancer cell lines (CaSki and CUMC-6). As shown in FIG. 31, it was revealed that the expression level of the TRG18 protooncogene was increased, that is, a dominant TRG18 mRNA transcript having a size of approximately 2.4 kb was overexpressed in the cervical cancer tissue and the cervical cancer cell lines CaSki and CUMC-6. In FIG. 31, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 31 shows the northern blotting result indicating whether or not β-actin mRNA is transcribed by hybridizing the same sample with β-actin probe.

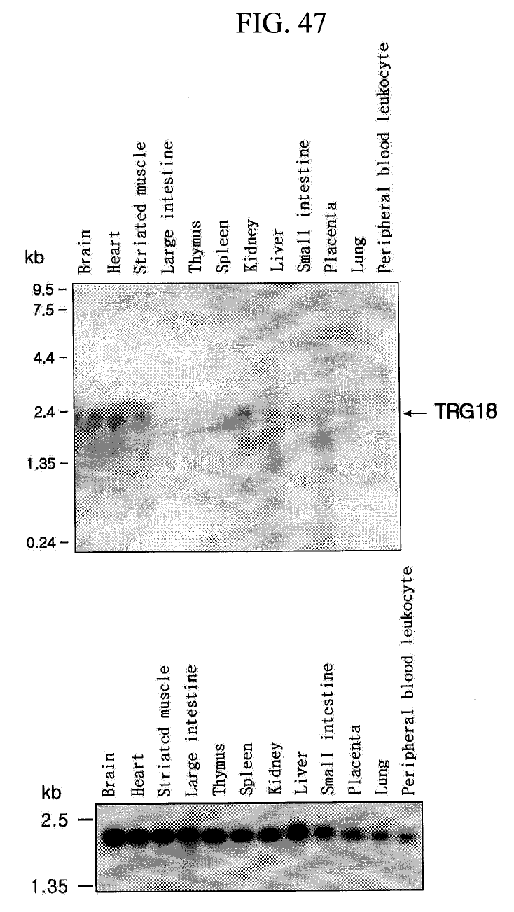

FIG. 47 shows a northern blotting result to determine whether or not the TRG18 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 47 shows the northern blotting result indicating whether or not β-actin mRNA is transcribed by hybridizing the same sample with β-actin probe. As shown in FIG. 47, it was revealed that a TRG18 mRNA transcript (the dominant TRG18 mRNA transcript having a size of approximately 2.4 kb) was rarely expressed in the normal tissues such as the brain, the heart, the muscle, the kidney, the liver and the placenta, but not expressed in the normal tissues such as the large intestine, the thymus, the spleen, the small intestine, the lung and the peripheral blood leukocyte. Also, a TRG18 mRNA transcript having a size of approximately 1.5 kb was very rarely expressed at the same time.

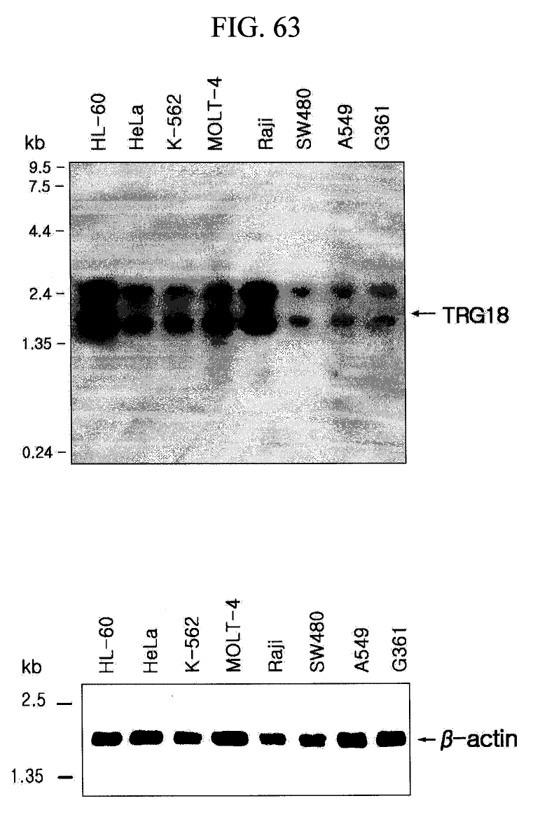

FIG. 63 shows a northern blotting result to determine whether or not the TRG18 protooncogene is expressed in the human cancer cell lines, for example HL-60, HeLa, K-562, MOLT-4, Raji, SW480, A549 and G361 (Clontech). A bottom of FIG. 63 shows the northern blotting result indicating whether or not β-actin mRNA is transcribed by hybridizing the same sample with β-actin probe. As shown in FIG. 63, it was revealed that a TRG18 mRNA transcript (the dominant TRG18 mRNA transcript having a size of approximately 2.4 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. Also, a TRG18 mRNA transcript having a size of approximately 1.5 kb was highly expressed at the same time.

A top of FIG. 32 shows a northern blotting result to determine whether or not the TRG20 protooncogene is expressed in the normal exocervical tissue, the cervical cancer tissue, the metastatic cervical lymph node tissue and the cervical cancer cell lines (CaSki and CUMC-6). As shown in FIG. 32, it was revealed that the expression level of the TRG20 protooncogene was increased, that is, a dominant TRG20 mRNA transcript having a size of approximately 1.3 kb was overexpressed in the cervical cancer tissue and the cervical cancer cell lines CaSki and CUMC-6. In FIG. 32, a lane “Normal” represents the normal exocervical tissue, a lane “Cancer” represents the cervical cancer tissue, a lane “metastasis” represents the metastatic cervical lymph node tissue, and each of lanes “CaSki” and “CUMC-6” represents the uterine cancer cell line. A bottom of FIG. 32 shows the northern blotting result indicating whether or not β-actin mRNA is transcribed by hybridizing the same sample with β-actin probe.

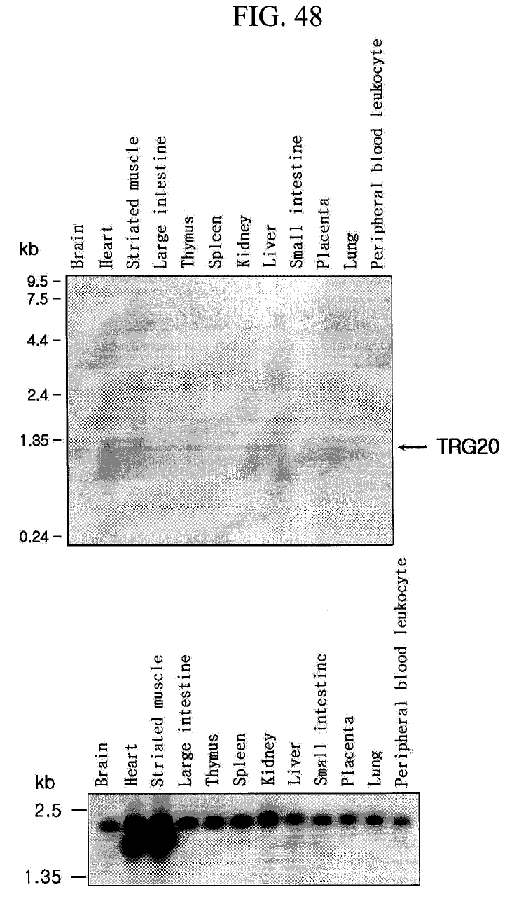

FIG. 48 shows a northern blotting result to determine whether or not the TRG20 protooncogene is expressed in the normal human 12-lane multiple tissues (Clontech), for example brain, heart, striated muscle, large intestine, thymus, spleen, kidney, liver, small intestine, placenta, lungs and peripheral blood leukocyte tissues. A bottom of FIG. 48 shows the northern blotting result indicating whether or not β-actin mRNA is transcribed by hybridizing the same sample with β-actin probe. As shown in FIG. 48, it was revealed that a TRG20 mRNA transcript (the dominant TRG20 mRNA transcript having a size of approximately 1.3 kb) was very rarely expressed or not expressed in the normal tissues such as the brain, the heart, the muscle, the large intestine, the thymus, the spleen, the kidney, the liver, the small intestine, the placenta, the lung and the peripheral blood leukocyte.

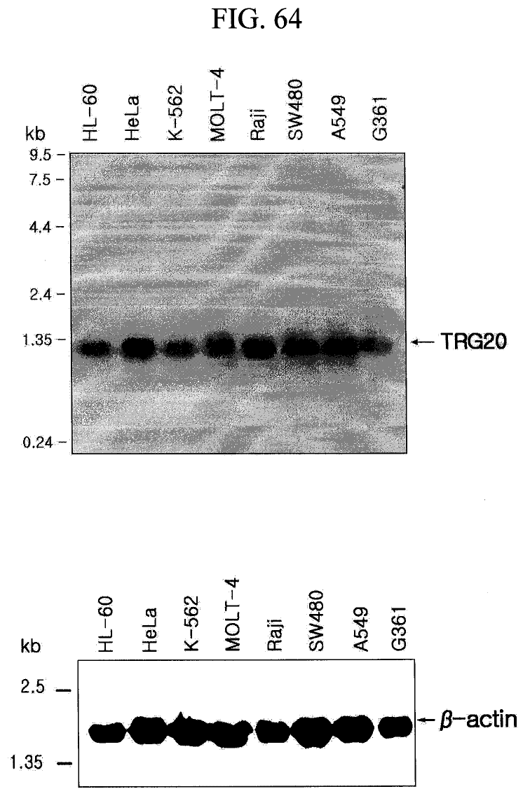

FIG. 64 shows a northern blotting result to determine whether or not the TRG20 protooncogene is expressed in the human cancer cell lines, for example HL-60, HeLa, K-562, MOLT-4, Raji, SW480, A549 and G361 (Clontech). A bottom of FIG. 64 shows the northern blotting result indicating whether or not β-actin mRNA is transcribed by hybridizing the same sample with β-actin probe. As shown in FIG. 64, it was revealed that a TRG20 mRNA transcript (the dominant TRG20 mRNA transcript having a size of approximately 1.3 kb) was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

### Example 8

### Size Determination of Protein Expressed after Transforming E. coli with TRG Protooncogene

Each of the TRG protooncogenes of SEQ ID NO: 1; SEQ ID NO: 5; SEQ ID NO: 9; SEQ ID NO: 13; SEQ ID NO: 17; SEQ ID NO: 21; SEQ ID NO: 25; SEQ ID NO: 29; SEQ ID NO: 33; SEQ ID NO: 37; SEQ ID NO: 41; SEQ ID NO: 45; SEQ ID NO: 49; SEQ ID NO: 53; SEQ ID NO: 57; and SEQ ID NO: 61 was inserted into a multi-cloning site of the pBAD/thio-Topo vector (Invitrogen, U.S.), and then E. coli Top10 (Invitrogen, U.S.) was transformed with each of the resultant pBAD/thio-Topo/TRG vectors. An expression protein, HT-Thioredoxin, is inserted in an upstream region of the multi-cloning site of the pBAD/thio-Topo vector. Each of the transformed E. coli strains was incubated in LB broth while shaking, and then each of the resultant cultures was diluted at a ratio of 1/100 and incubated for 3 hours again. 0.5 mM L-Arabinose (Sigma) was added thereto to facilitate production of their proteins.

The E. coli cells were sonicated in the culture media before/after L-Arabinose induction, and then the sonicated homogenates were subject to 12% sodium dodecyl sulfate-polyacrylamide gel electrophoresis (SDS-PAGE).

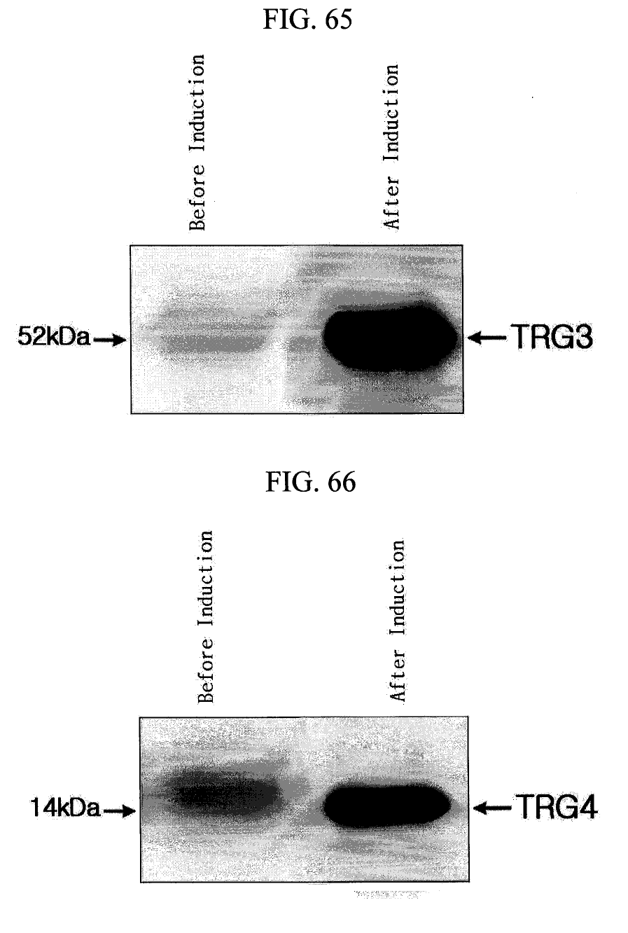

FIG. 65 shows a SDS-PAGE result to determine an expression pattern of proteins in the E. coli Top 10 strain transformed with the pBAD/thio-Topo/TRG3 vector, wherein a band of a fusion protein having a molecular weight of approximately 67 kDa was clearly observed after L-arabinose induction. The 67-kDa fusion protein includes the HT-thioredoxin protein having a molecular weight of approximately 15 kDa and the TRG3 protein having a molecular weight of approximately 52 kDa, each protein being inserted into the pBAD/thio-Topo/TRG3 vector.

FIG. 66 shows a SDS-PAGE result to determine an expression pattern of proteins in the E. coli Top10 strain transformed with the pBAD/thio-Topo/TRG4 vector, wherein a band of a fusion protein having a molecular weight of approximately 29 kDa was clearly observed after L-arabinose induction. The 29-kDa fusion protein includes the HT-thioredoxin protein having a molecular weight of approximately 15 kDa and the TRG4 protein having a molecular weight of approximately 14 kDa, each protein being inserted into the pBAD/thio-Topo/TRG4 vector.

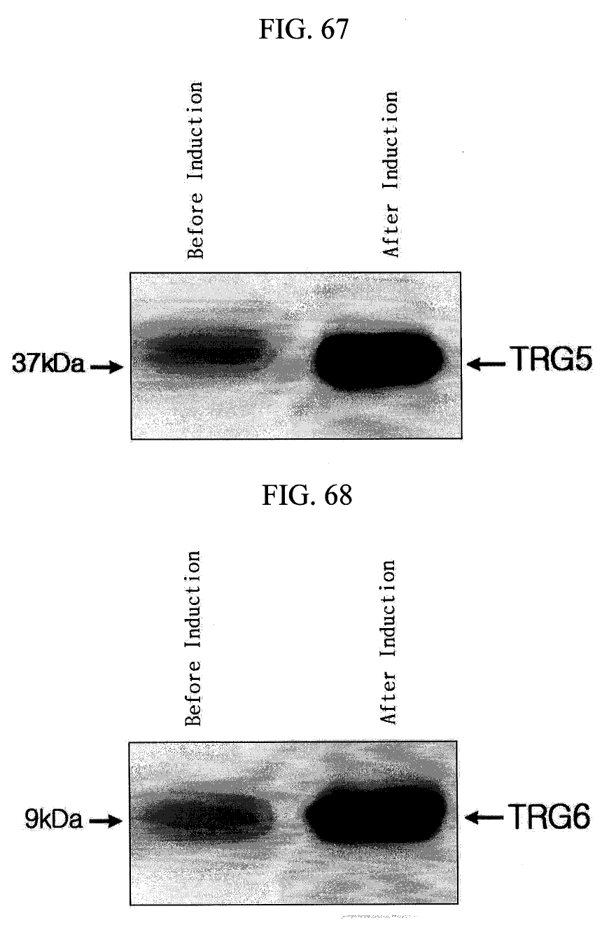

FIG. 67 shows a SDS-PAGE result to determine an expression pattern of proteins in the E. coli Top 10 strain transformed with the pBAD/thio-Topo/TRG5 vector, wherein a band of a fusion protein having a molecular weight of approximately 52 kDa was clearly observed after L-arabinose induction. The 52-kDa fusion protein includes the HT-thioredoxin protein having a molecular weight of approximately 15 kDa and the TRG5 protein having a molecular weight of approximately 37 kDa, each protein being inserted into the pBAD/thio-Topo/TRG5 vector.

FIG. 68 shows a SDS-PAGE result to determine an expression pattern of proteins in the E. coli Top10 strain transformed with the pBAD/thio-Topo/TRG6 vector, wherein a band of a fusion protein having a molecular weight of approximately 24 kDa was clearly observed after L-arabinose induction. The 24-kDa fusion protein includes the HT-thioredoxin protein having a molecular weight of approximately 15 kDa and the TRG6 protein having a molecular weight of approximately 9 kDa, each protein being inserted into the pBAD/thio-Topo/TRG6 vector.

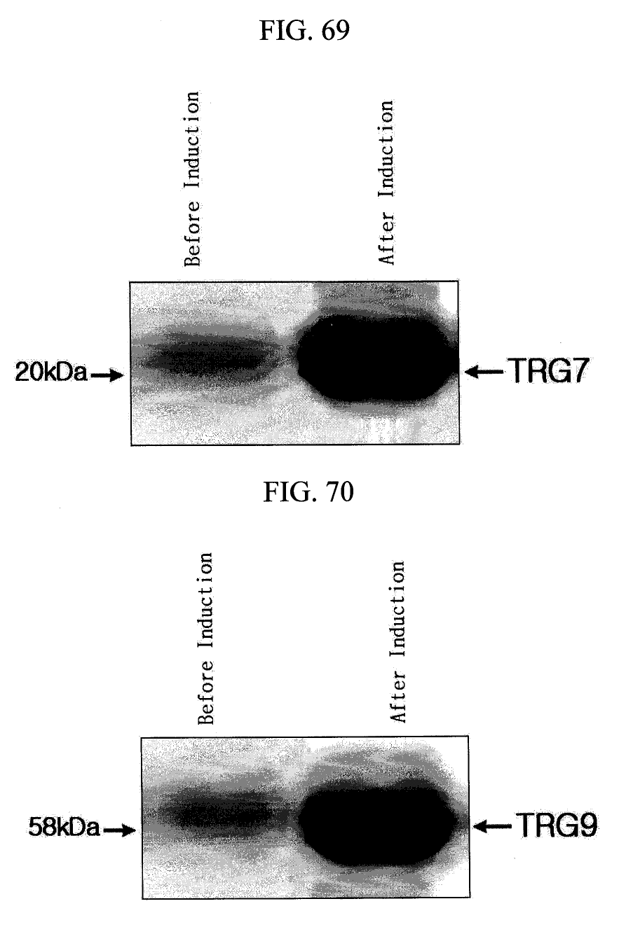

FIG. 69 shows a SDS-PAGE result to determine an expression pattern of proteins in the E. coli Top 10 strain transformed with the pBAD/thio-Topo/TRG7 vector, wherein a band of a fusion protein having a molecular weight of approximately 35 kDa was clearly observed after L-arabinose induction. The 35-kDa fusion protein includes the HT-thioredoxin protein having a molecular weight of approximately 15 kDa and the TRG7 protein having a molecular weight of approximately 20 kDa, each protein being inserted into the pBAD/thio-Topo/TRG7 vector.

FIG. 70 shows a SDS-PAGE result to determine an expression pattern of proteins in the E. coli Top10 strain transformed with the pBAD/thio-Topo/TRG9 vector, wherein a band of a fusion protein having a molecular weight of approximately 73 kDa was clearly observed after L-arabinose induction. The 73-kDa fusion protein includes the HT-thioredoxin protein having a molecular weight of approximately 15 kDa and the TRG9 protein having a molecular weight of approximately 58 kDa, each protein being inserted into the pBAD/thio-Topo/TRG9 vector.

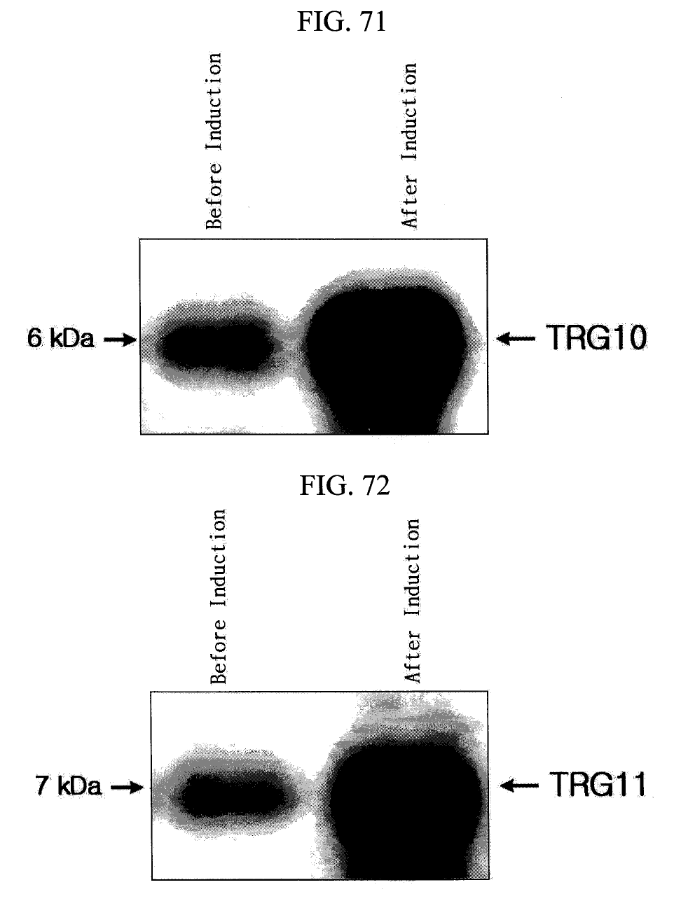

FIG. 71 shows a SDS-PAGE result to determine an expression pattern of proteins in the E. coli Top10 strain transformed with the pBAD/thio-Topo/TRG10 vector, wherein a band of a fusion protein having a molecular weight of approximately 21 kDa was clearly observed after L-arabinose induction. The 21-kDa fusion protein includes the HT-thioredoxin protein having a molecular weight of approximately 15 kDa and the TRG10 protein having a molecular weight of approximately 6 kDa, each protein being inserted into the pBAD/thio-Topo/TRG10 vector.

FIG. 72 shows a SDS-PAGE result to determine an expression pattern of proteins in the E. coli Top10 strain transformed with the pBAD/thio-Topo/TRG11 vector, wherein a band of a fusion protein having a molecular weight of approximately 22 kDa was clearly observed after L-arabinose induction. The 22-kDa fusion protein includes the HT-thioredoxin protein having a molecular weight of approximately 15 kDa and the TRG11 protein having a molecular weight of approximately 7 kDa, each protein being inserted into the pBAD/thio-Topo/TRG11 vector.

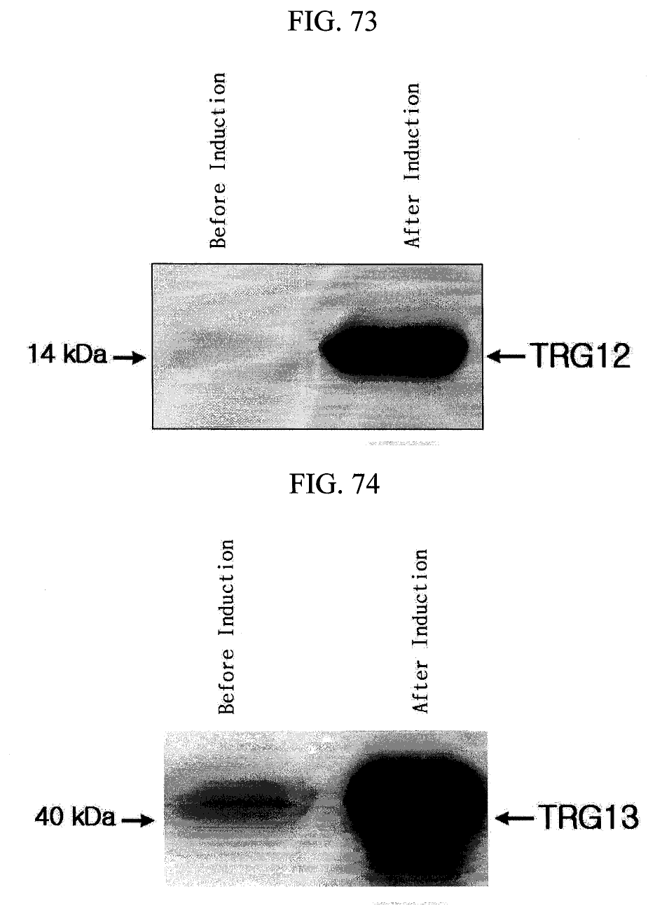

FIG. 73 shows a SDS-PAGE result to determine an expression pattern of proteins in the E. coli Top10 strain transformed with the pBAD/thio-Topo/TRG12 vector, wherein a band of a fusion protein having a molecular weight of approximately 29 kDa was clearly observed after L-arabinose induction. The 29-kDa fusion protein includes the HT-thioredoxin protein having a molecular weight of approximately 15 kDa and the TRG12 protein having a molecular weight of approximately 14 kDa, each protein being inserted into the pBAD/thio-Topo/TRG12 vector.

FIG. 74 shows a SDS-PAGE result to determine an expression pattern of proteins in the E. coli Top10 strain transformed with the pBAD/thio-Topo/TRG13 vector, wherein a band of a fusion protein having a molecular weight of approximately 55 kDa was clearly observed after L-arabinose induction. The 55-kDa fusion protein includes the HT-thioredoxin protein having a molecular weight of approximately 15 kDa and the TRG13 protein having a molecular weight of approximately 40 kDa, each protein being inserted into the pBAD/thio-Topo/TRG13 vector.

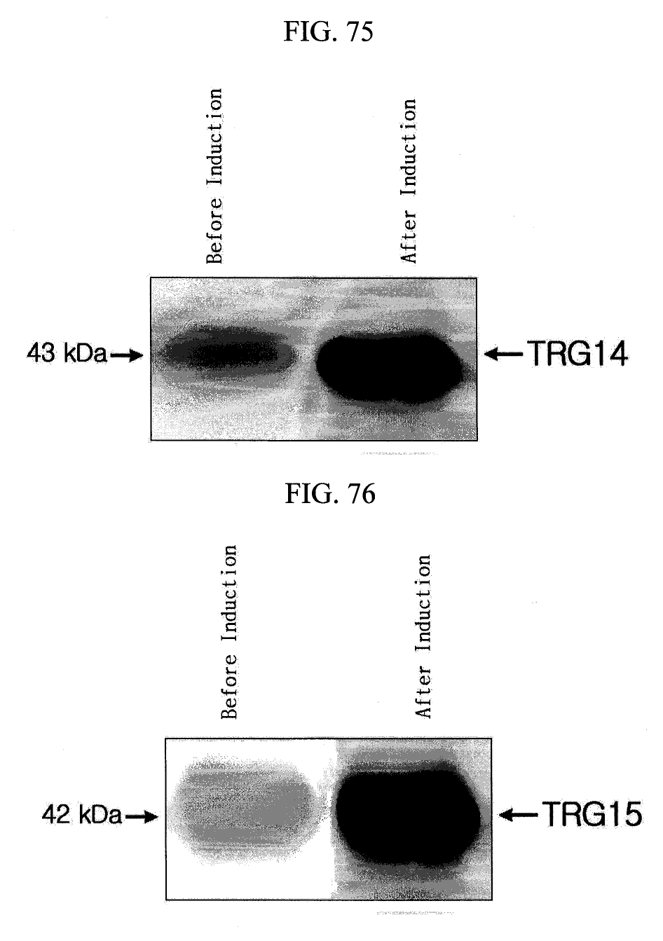

FIG. 75 shows a SDS-PAGE result to determine an expression pattern of proteins in the E. coli Top10 strain transformed with the pBAD/thio-Topo/TRG14 vector, wherein a band of a fusion protein having a molecular weight of approximately 58 kDa was clearly observed after L-arabinose induction. The 58-kDa fusion protein includes the HT-thioredoxin protein having a molecular weight of approximately 15 kDa and the TRG14 protein having a molecular weight of approximately 43 kDa, each protein being inserted into the pBAD/thio-Topo/TRG14 vector.

FIG. 76 shows a SDS-PAGE result to determine an expression pattern of proteins in the E. coli Top10 strain transformed with the pBAD/thio-Topo/TRG15 vector, wherein a band of a fusion protein having a molecular weight of approximately 57 kDa was clearly observed after L-arabinose induction. The 57-kDa fusion protein includes the HT-thioredoxin protein having a molecular weight of approximately 15 kDa and the TRG15 protein having a molecular weight of approximately 42 kDa, each protein being inserted into the pBAD/thio-Topo/TRG15 vector.

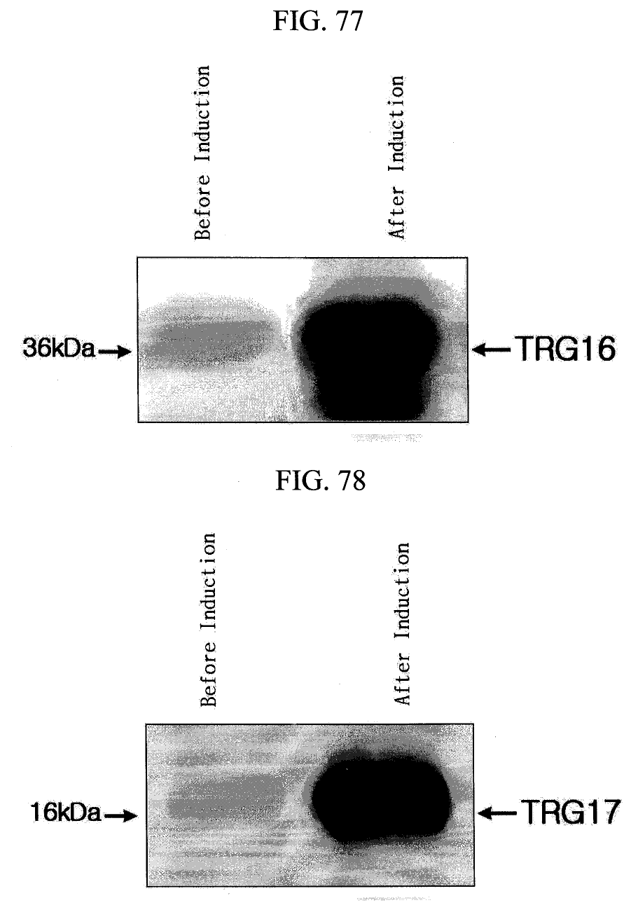

FIG. 77 shows a SDS-PAGE result to determine an expression pattern of proteins in the E. coli Top10 strain transformed with the pBAD/thio-Topo/TRG16 vector, wherein a band of a fusion protein having a molecular weight of approximately 51 kDa was clearly observed after L-arabinose induction. The 51-kDa fusion protein includes the HT-thioredoxin protein having a molecular weight of approximately 15 kDa and the TRG16 protein having a molecular weight of approximately 36 kDa, each protein being inserted into the pBAD/thio-Topo/TRG16 vector.

FIG. 78 shows a SDS-PAGE result to determine an expression pattern of proteins in the E. coli Top10 strain transformed with the pBAD/thio-Topo/TRG17 vector, wherein a band of a fusion protein having a molecular weight of approximately 31 kDa was clearly observed after L-arabinose induction. The 31-kDa fusion protein includes the HT-thioredoxin protein having a molecular weight of approximately 15 kDa and the TRG17 protein having a molecular weight of approximately 16 kDa, each protein being inserted into the pBAD/thio-Topo/TRG17 vector.

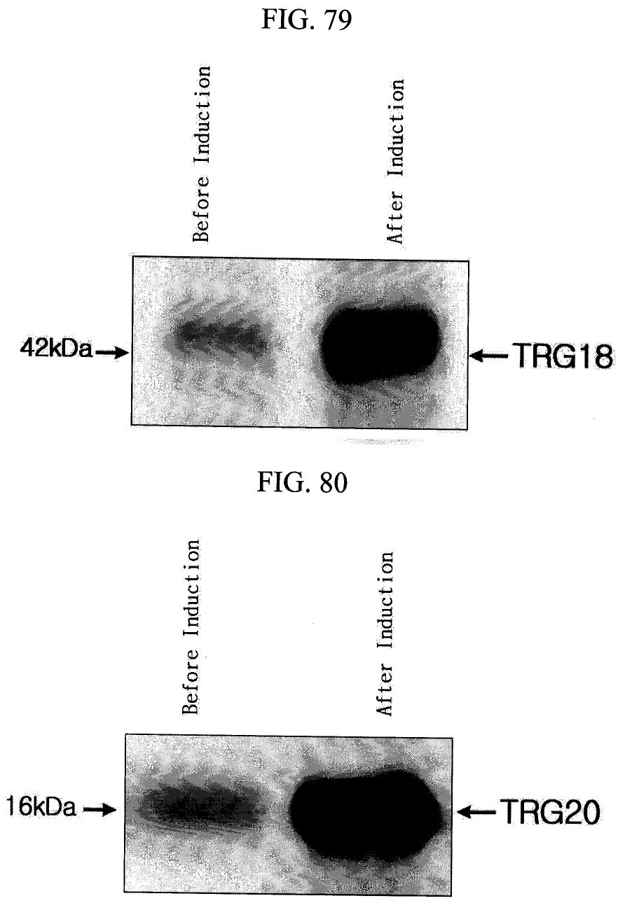

FIG. 79 shows a SDS-PAGE result to determine an expression pattern of proteins in the E. coli Top10 strain transformed with the pBAD/thio-Topo/TRG18 vector, wherein a band of a fusion protein having a molecular weight of approximately 57 kDa was clearly observed after L-arabinose induction. The 57-kDa fusion protein includes the HT-thioredoxin protein having a molecular weight of approximately 15 kDa and the TRG18 protein having a molecular weight of approximately 42 kDa, each protein being inserted into the pBAD/thio-Topo/TRG18 vector.

FIG. 80 shows a SDS-PAGE result to determine an expression pattern of proteins in the E. coli Top10 strain transformed with the pBAD/thio-Topo/TRG20 vector, wherein a band of a fusion protein having a molecular weight of approximately 31 kDa was clearly observed after L-arabinose induction. The 31-kDa fusion protein includes the HT-thioredoxin protein having a molecular weight of approximately 15 kDa and the TRG20 protein having a molecular weight of approximately 16 kDa, each protein being inserted into the pBAD/thio-Topo/TRG20 vector.

### INDUSTRIAL APPLICABILITY

As described above, the protooncogene of the present invention, known to be involved in human carcinogenesis and simultaneously exhibit an ability to induce cancer metastasis, may be effectively used for diagnosing various cancers including uterine cancer, leukemia, lymphoma, colon cancer, lung cancer, skin cancer, etc.

1. A human protooncoprotein having an amino acid sequence selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; SEQ ID NO: 34; SEQ ID NO: 38; SEQ ID NO: 42; SEQ ID NO: 46; SEQ ID NO: 50; SEQ ID NO: 54; SEQ ID NO: 58; and SEQ ID NO: 62.

2. A human protooncogene encoding the protooncoprotein defined in claim 1.

3. The human protooncogene according to claim 2, comprising a DNA sequence selected from the group consisting of a DNA sequence corresponding to nucleotide sequence positions from 113 to 1,522 of SEQ ID NO: 1; a DNA sequence corresponding to nucleotide sequence positions from 87 to 482 of SEQ ID NO: 5; a DNA sequence corresponding to nucleotide sequence positions from 88 to 1,092 of SEQ ID NO: 9; a DNA sequence corresponding to nucleotide sequence positions from 233 to 481 of SEQ ID NO: 13; a DNA sequence corresponding to nucleotide sequence positions from 42 to 1,422 of SEQ ID NO: 17; a DNA sequence corresponding to nucleotide sequence positions from 17 to 1,576 of SEQ ID NO: 21; a DNA sequence corresponding to nucleotide sequence positions from 1100 to 1,270 of SEQ ID NO: 25; a DNA sequence corresponding to nucleotide sequence positions from 26 to 214 of SEQ ID NO: 29; a DNA sequence corresponding to nucleotide sequence positions from 80 to 475 of SEQ ID NO: 33; a DNA sequence corresponding to nucleotide sequence positions from 18 to 1,193 of SEQ ID NO: 37; a DNA sequence corresponding to nucleotide sequence positions from 18 to 1,202 of SEQ ID NO: 41; a DNA sequence corresponding to nucleotide sequence positions from 1 to 1,104 of SEQ ID NO: 45; a DNA sequence corresponding to nucleotide sequence positions from 92 to 1,064 of SEQ ID NO: 49; a DNA sequence corresponding to nucleotide sequence positions from 1 to 408 of SEQ ID NO: 53; a DNA sequence corresponding to nucleotide sequence positions from 20 to 1,141 of SEQ ID NO: 57; and a DNA sequence corresponding to nucleotide sequence positions from 42 to 449 of SEQ ID NO: 61.

4. The human protooncogene according to claim 2, having a DNA sequence selected from the group consisting of SEQ ID NO: 1; SEQ ID NO: 5; SEQ ID NO: 9; SEQ ID NO: 13; SEQ ID NO: 17; SEQ ID NO: 21; SEQ ID NO: 25; SEQ ID NO: 29; SEQ ID NO: 33; SEQ ID NO: 37; SEQ ID NO: 41; SEQ ID NO: 45; SEQ ID NO: 49; SEQ ID NO: 53; SEQ ID NO: 57; and SEQ ID NO: 61.

5. A vector including the protooncogene defined in claim 2.

6. A kit for diagnosing cancer including the protooncoprotein defined in claim 1.

7. A kit for diagnosing cancer including the protooncogene defined in claim 2.

8. A vector including the protooncogene defined in claim 3.

9. A vector including the protooncogene defined in claim 4.

10. A kit for diagnosing cancer including the protooncogene defined in claim 3.

11. A kit for diagnosing cancer including the protooncogene defined in claim 4.
